SEQUENCE LISTING (8 sequences)

SEQ ID NO: 1 - protein, 522 aa, Torpedo californica
  nAChR delta subunit
MGNIHFVYLLISCLYYSGCSGVNEEERLINDLLIVNKYNKHVRPVKHNNEVVNIALSLTLSNLISLKETDETLTSNVWMDHAWYDHRLTWNASEYSDISILRLPPELVWIPDIVLQNNNDGQYHVAYFCNVLVRPNGYVTWLPPAIFRSSCPINVLYFPFDWQNCSLKFTALNYDANEITMDLMTDTIDGKDYPIEWIIIDPEAFTENGEWEIIHKPAKKNIYPDKFPNGTNYQDVTFYLIIRRKPLFYVINFITPCVLISFLASLAFYLPAESGEKMSTAISVLLAQAVFLLLTSQRLPETALAVPLIGKYLMFIMSLVTGVIVNCGIVLNFHFRTPSTHVLSTRVKQIFLEKLPRILHMSRADESEQPDWQNDLKLRRSSSVGYISKAQEYFNIKSRSELMFEKQSERHGLVPRVTPRIGFGNNNENIAASDQLHDEIKSGIDSTNYIVKQIKEKNAYDEEVGNWNLVGQTIDRLSMFIITPVMVLGTIFIFVMGNFNHPPAKPFEGDPFDYSSDHPRCA

SEQ ID NO: 2 - protein, 506 aa, Torpedo californica
  nAChR gamma subunit
MVLTLLLIICLALEVRSENEEGRLIEKLLGDYDKRIIPAKTLDHIIDVTLKLTLTNLISLNEKEEALTTNVWIEIQWNDYRLSWNTSEYEGIDLVRIPSELLWLPDVVLENNVDGQFEVAYYANVLVYNDGSMYWLPPAIYRSTCPIAVTYFPFDWQNCSLVFRSQTYNAHEVNLQLSAEEGEAVEWIHIDPEDFTENGEWTIRHRPAKKNYNWQLTKDDTDFQEIIFFLIIQRKPLFYIINIIAPCVLISSLVVLVYFLPAQAGGQKCTLSISVLLAQTIFLFLIAQKVPETSLNVPLIGKYLIFVMFVSMLIVMNCVIVLNVSLRTPNTHSLSEKIKHLFLGFLPKYLGMQLEPSEETPEKPQPRRRSSFGIMIKAEEYILKKPRSELMFEEQKDRHGLKRVNKMTSDIDIGTTVDLYKDLANFAPEIKSCVEACNFIAKSTKEQNDSGSENENWVLIGKVIDKACFWIALLLFSIGTLAIFLTGHFNQVPEFPFPGDPRKYVP

SEQ ID NO: 3 - protein, 493 aa, Torpedo californica
  nAChR beta subunit
MEDVRRMALGVVVMMALALSGVGASVMEDTLLSVLFETYNPKVRPAQTVGDKVTVRVGLTLTNLLILNEKIEEMTTNVFLNLAWTDYRLQWDPAAYEGIKDLRIPSSDVWQPDIVLMNNNDGSFEITLHVNVLVQHTGAVSWQPSAIYRSSCTIKVMYFPFDWQNCTMVFKSYTYDTSEVTLQHALDAKGEREVKEIVINKDAFTENGQWSIEHKPSRKNWRSDDPSYEDVTFYLIIQRKPLFYIVYTIIPCILISILAILVFYLPPDAGEKMSLSISALLAVTVFLLLLADKVPETSLSVPIIIRYLMFIMILVAFSVILSVVVLNLHHRSPNTHTMPNWIRQIFIETLPPFLWIQRPVTTPSPDSKPTIISRANDEYFIRKPAGDFVCPVDNARVAVQPERLFSEMKWHLNGLTQPVTLPQDLKEAVEAIKYIAEQLESASEFDDLKKDWQYVAMVADRLFLYVFFVICSIGTFSIFLDASHNVPPDNPFA

SEQ ID NO: 4 - protein, 461 aa, Torpedo californica
  nAChR alpha subunit
MILCSYWHVGLVLLLFSCCGLVLGSEHETRLVANLLENYNKVIRPVEHHTHFVDITVGLQLIQLISVDEVNQIVETNVRLRQQWIDVRLRWNPADYGGIKKIRLPSDDVWLPDLVLYNNADGDFAIVHMTKLLLDYTGKIMWTPPAIFKSYCEIIVTHFPFDQQNCTMKLGIWTYDGTKVSISPESDRPDLSTFMESGEWVMKDYRGWKHWVYYTCCPDTPYLDITYHFIMQRIPLYFVVNVIIPCLLFSFLTGLVFYLPTDSGEKMTLSISVLLSLTVFLLVIVELIPSTSSAVPLIGKYMLFTMIFVISSIIITVVVINTHHRSPSTHTMPQWVRKIFIDTIPNVMFFSTMKRASKEKQENKIFADDIDISDISGKQVTGEVIFQTPLIKNPDVKSAIEGVKYIAEHMKSDEESSNAAEEWKYVAMVIDHILLCVFMLICIIGTVSVFAGRLIELSQEG

SEQ ID NO: 5 - protein, 12 aa, Torpedo californica
  Delta subunit, nAChR Residues 299-311(M2-M3 loop)
LPETALAVPLIG

SEQ ID NO: 6 - protein, 10 aa, Torpedo californica
  Alpha subunit, nAChR Residues 170-179 (ligand binding domain)
LGIWTYDGTK

SEQ ID NO: 7 - protein, 24 aa, Torpedo californica
  Alpha subunit, nAChR Residues 180-203 (ligand binding domain)
VSISPESDRPDLSTFMESGEWVMK

SEQ ID NO: 8 - protein, 497 aa, Artificial sequence [sequence omitted]

(19) United States
(12) Patent Application Publication
(10) Pub. No.: US 2011/0315930 A1
(43) Pub. Date: Dec. 29, 2011

(54) Compositions and methods for analyzing biomolecules using mass spectroscopy

(75) Inventors: Robert Marshall Pope, San Marcos, CA (US); John Leite, Vista, CA (US); Mahbod Hajivandi, Vista, CA (US); Charles George Shevlin, San Diego, CA (US); Timothy Updyke, Temecula, CA (US)

(73) Assignee: LIFE TECHNOLOGIES CORPORATION, Carlsbad, CA (US)

(21) Appl. No.: 12/970,762

(22) Filed: Dec. 16, 2010

Related U.S. Application Data
(63) Continuation of application No. 12/553,017, filed on Sep. 2, 2009, now abandoned.
Application No. 12/553,017 is a continuation of application No. 11/258,363, filed on Oct. 26, 2005, now abandoned.
(60) Provisional application No. 60/621,685, filed on Oct. 26, 2004.
(60) Provisional application No. 60/621,686, filed on Oct. 26, 2004.
(60) Provisional application No. 60/669,373, filed on Apr. 8, 2005.
(60) Provisional application No. 60/685,869, filed on Jun. 1, 2005.

(51) Int. Cl. G01N 33/00; B01F 1/00

(52) U.S. Cl. 252/364; 252/408.1

(57) ABSTRACT

Compositions and methods for mass spectroscopy are disclosed. The compositions and methods relate to the analysis of proteins and other biopolymers using mass spectroscopy, particularly matrix-assisted laser desorption time-of-flight mass spectrometry (MALDI-TOF MS).

This application claims benefit of priority to U.S. Provisional Application No. 60/621,685, filed Oct. 26, 2004; U.S. Provisional Application No. 60/621,686, filed Oct. 26, 2004; U.S. Application Provisional No. 60/669,373, filed Apr. 8, 2005; and U.S. Provisional Application No. 60/685,869 filed Jun. 1, 2005; all of which are entitled “Compositions and Methods for Analyzing Biomolecules Using Mass Spectroscopy” and incorporated by reference herein in their entireties.

### TECHNICAL FIELD OF THE INVENTION

The present invention relates to the analysis of proteins and other biopolymers using mass spectroscopy (MS), particularly for matrix-assisted laser desorption time-of-flight mass spectrometry (MALDI-TOF MS) and liquid chromatography mass spectrometry (LC/MS).

### BACKGROUND OF THE INVENTION

In various aspects, the invention is drawn to mass spectroscopy. As used herein, the term “mass spectrometry” (or simply “MS”) encompasses any spectrometric technique or process in which molecules are ionized and separated and/or analyzed based on their respective molecular weights. Thus, as used herein, the terms “mass spectrometry” and “MS” encompass any type of ionization method, including without limitation electrospray ionization (ESI), atmospheric-pressure chemical ionization (APCI) and other forms of atmospheric pressure ionization (API), and laser irradiation. Mass spectrometers are commonly combined with separation methods such as gas chromatography (GC) and liquid chromatography (LC). The GC or LC separates the components in a mixture, and the components are then individually introduced into the mass spectrometer; such techniques are generally called GC/MS and LC/MS, respetively. MS/MS is an analogous technique where the first-stage separation device is another mass spectrometer. In LC/MS/MS, the separation methods comprise liquid chromatography and MS. Any combination (e.g., GC/MS/MS, GC/LC/MS, GC/LC/MS/MS, etc.) of methods can be used to practice the invention. In such combinations, “MS” can refer to any form of mass spectrtometry; by way of non-limiting example, “LC/MS” encompasses LC/ESI MS and LC/MALDI-TOF MS. Thus, as used herein, the terms “mass spectrometry” and “MS” include without limitation APCI MS; ESI MS; GC MS; MALDI-TOF MS; LC/MS combinations; LC/MS/MS combinations; MS/MS combinations; etc.

### HPLC and RP-HPLC

It is often necessary to prepare samples comprising an analyte of interest for MS. Such preparations include without limitation purifcation and/or buffer exchange. Any appropriate method, or combination of methods, can be used to prepare samples for MS. One preferred type of MS preparative method is liquid chromatography (LC), including without limitation HPLC and RP-HPLC.

High-pressure liquid chromatography (HPLC) is a separative and quantitative analytical tool that is generally robust, reliable and flexible. Reverse-phase (RP) is a commonly used stationary phase that is characterized by alkyl chains of specific length immobilized to a silica bead support. RP-HPLC is suitable for the separation and analysis of various types of compounds including without limitation biomolecules, (e.g., carbohydrates, proteins, peptides, and nucleic acids). One of the most important reasons that RP-HPLC has been the technique of choice amongst all HPLC techniques is its compatibility with electrospray ionization (ESI). During ESI, liquid samples can be introduced into a mass spectrometer by a process that creates multiple charged ions (Wilm et al., Anal. Chem. 68:1, 1996). However, multiple ions can result in complex spectra and reduced sensitivity.

In HPLC, peptides and proteins are injected into a column, typically silica based C18. An aqueous buffer is used to elute the salts, while the peptides and proteins are eluted with a mixture of aqueous solvent (water) and organic solvent (acetonitrile, methanol, propanol). The aqueous phase is generally HPLC grade water with 0.1% acid and the organic solvent phase is generally an HPLC grade acetonitrile or methanol with 0.1% acid. The acid is used to improve the chromatographic peak shape and to provide a source of protons in reverse phase LC/MS. The acids most commonly used are formic acid, triflouroacetic acid, and acetic acid. In RP HPLC, compounds are separated based on their hydrophobic character. With an LC system coupled to the mass spectrometer through an ESI source and the ability to perform data-dependant scanning, it is now possible in at least some instances to distinguish proteins in complex mixtures containing more than 50 components without first purifying each protein to homogeneity.

### MALDI-TOF MS

A particular type of MS technique, matrix-assisted laser desorption time-of-flight mass spectrometry (MALDI-TOF MS) (Karas et al., Int. J. Mass Spectrom. Ion Processes 78:53, 1987), has received prominence in analysis of biological polymers for its desirable characteristics, such as relative ease of sample preparation, predominance of singly charged ions in mass spectra, sensitivity and high speed. MALDI-TOF MS is a technique in which a UV-light absorbing matrix and a molecule of interest (analyte) are mixed and co-precipitated, thus forming analyte:matrix crystals. The crystals are irradiated by a nanosecond laser pulse. Most of the laser energy is absorbed by the matrix, which prevents unwanted fragmentation of the biomolecule. Matrix molecules transfer their energy to analyte molecules, causing them to vaporize and ionize. The ionized molecules are accelerated in an electric field and enter the flight tube. During the flight in this tube, different molecules are separated according to their mass to charge (m/z) ratio and reach the detector at different times. Each molecule yields a distinct signal. The method is used for detection and characterization of biomolecules, such as proteins, peptides, oligosaccharides and oligonucleotides, with molecular masses between about 400 and about 500,000 Da, or higher. MALDI-MS is a sensitive technique that allows the detection of low (10-15 to 10-18 mole) quantities of analyte in a sample.

Partial amino acid sequences of proteins can be determined by enzymatic proteolysis followed by MS analysis of the product peptides. These amino acid sequences can be used for in silico examination of DNA and/or protein sequence databases. Matched amino acid sequences can indicate proteins, domains and/or motifs having a known function and/or tertiary structure. For example, amino acid sequences from an uncharacterized protein might match the sequence or structure of a domain or motif that binds a ligand. As another example, the amino acid sequences can be used in vitro as antigens to generate antibodies to the protein and other related proteins from other biological source material (e.g., from a different tissue or organ, or from another species). There are many additional uses for MS, particularly MALDI-TOF MS, in the fields of genomics, proteomics and drug discovery. For a general review of the use of MALDI-TOF MS in proteomics and genomics, see Bonk et al. (Neuroscientist 7:12, 2001).

Although MALDI-TOF MS is a powerful technique, it has its limitations. Non-limiting examples of such limitations involve adduction, solubilization, and a limited analyzable surface area. These and other factors increase the amount of “noise” in MS spectra.

### Adduction

One limitation to MALDI-MS is the process of adduction, in which ions form adducts that interfere with MALDI-TOF mass spectroscopy. For example, sodium, potassium, ammonium and other monovalent cations are known to cause adducts that interfere with MALDI-TOF mass spectroscopy, generally when present in a range of from about 10 to about 750 mM, more specifically from about 50 to about 500 mM. Protein adducts are particularly undesirable to those studying a proteome: the transformation and loss of molecules of interest results in the production of adducts, which are undesirable contaminants. Both events complicate the target sample, and both can introduce inaccuracy and/or imprecision in the MS spectra.

In MALDI-TOF MS studies of samples, sample complexity can result in less sensitive and accurate results. Sample complexity reflects a number of factors but it generally increases as the number of different molecular species in a sample increases and as the concentration of undesirable molecular species (i.e., molecules other than the molecule of interest) increases. One source of sample complexity is adduction of monovalent cations to peptides. This leads to the formation of peptide:ion adducts with ions. For example, monovalent cations such as sodium and potassium ions are undesirable contaminants that originate from commonly used buffers or from incompletely deionized water. During MALDI-TOF MS analysis, these cations can associate with peptides and cause the formation of adduct clusters in the spectrum. The adduct cluster peaks repeat at intervals of (M−1) Da, where M is the molecular mass of the cation. Thus, the formation of peptide:ion adducts increases the sample complexity, as it introduces several new molecular species into the sample.

The presence of cation adduct clusters in MALDI-MS spectra can easily complicate a peptide mass fingerprint analysis. Adducts reduce the sensitivity of the analysis by partitioning the signal intensity arising from a single peptide into various adduct cluster peaks. This phenomenon is especially problematic for enzymatic digests of low abundance proteins. Adduct clusters can also suppress the signal of an overlapping or neighboring peak of low abundance. In the extreme this could result in lower confidence for protein identification resulting in missed protein identifications and/or lower sequence coverage. Identification of a specific post-translational modification (PTM) is predicated upon the recognition of a unique mass difference between the observed peptide and the unaltered mass of the corresponding peptide as listed in an in silico digest. Monovalent cations can also preclude the characterization of PTMs.

There are some methods available for desalting samples prior to MALDI-MS analysis (Simpson, Proteins and Proteomics: A Laboratory Manual, Cold Spring Harbor Laboratory Press, Cold Spring Harbor, N.Y., 2003; see especially p. 454). Reverse-phase (RP) extraction using C18 beads in pipette tips is perhaps the most common method applied to protein digest samples. Although removal of cation adducts by this method is effective, it can contribute to loss of low abundance peptides (Tannu et al., Anal Biochem 327:222-232, 2004). Alternatively, peptides co-spotted and co-crystallized with MALDI matrix can be washed on the MALDI target using solvent or water to remove excess salts (Vorm et al., Anal. Chem. 66:3281, 1994), but this protocol can also result in significant loss of low abundance peptides. Also, samples can be desalted by reverse-phase separation via an HPLC where the eluted fractions are spotted (either manually or by a robotic device) onto MALDI target surfaces.

Another method is to displace monovalent metal cations with a volatile monovalent cation such as ammonium (Cheng et al., Rapid Commun Mass Spectrom 10:907, 1996), however this involves introduction of yet another salt into the sample, which may lead to overall signal suppression and extensive formation of matrix clusters.

There are some reports of adding ion-exchange beads directly to the MALDI matrix for removal of monovalent cation adduct clusters. This technique has been described for, for example, MALDI analysis of DNA (Harksen et al., Clinical Chemistry 45:1157-1161, 1999) and RNA (Tolson et al., Nucleic Acids Research 26:446-451, 1998).

### Solubilization

One limitation to the application of MALDI-MS is that solubilizing agents, useful in the analysis of hydrophobic analytes, interfere with MALDI-TOF MS and other types of mass spectroscopy. In proteomic studies, the analytes are proteins, some of which are readily soluble in water, some of which are not. There are, however, many challenges in the analysis of the hydrophobic proteins. Chief among these is the fact that they are, by definition, insoluble or only slightly soluble in water, and usually require the addition of one or more solubilizers in order to be used in many analytical techniques. Although there have been advances in the extraction, solubilization, chromatography and biochemical manipulation of hydrophobic proteins, the solubilization reagents used are largely incompatible with mass spectrometry analysis; they are not MS-compatible. The term “MS-compatible” as used herein generally indicates a composition that can be used in MALDI-TOF MS experiments. More specifically, a MS-compatible solubilizer preferably does not (1) interfere with the co-precipitation of analyte and matrix molecules, (2) impede the transfer of energy from matrix to analyte molecules, (3) lower the ionization efficiency of analyte molecules, and/or (4) increase the number of ion adducts. MS-compatible compositons and techniques for purifying analytes for MS, including without limitation MALDI-TOF MS, preferably have the further desirable characteristic of (5) not adhering or causing damage to the column and/or instrument tubing during and after appropriate washing procedures.

The predominant strategy involves removal of these solubilizing agents prior to analysis (see, e.g., Mock et al., Rapid Commun Mass Spectrom. 6:233, 1992). Removing the solubilizers is a time-consuming process, and does not always produce acceptable results. Attempts at MALDI-MS analysis of hydrophobic proteins have thus met with limited or partial success. This is particularly unfortunate with regards to proteomics studies, as hydrophobic proteins, including membrane proteins, constitute nearly half of the diversity of some proteomes.

MS-compatible solubilizers and other compositions, such as blends of detergents and/or non-detergent surfactants and blends (mixtures) thereof, that may be used to sequester monovalent cation adducts are disclosed herein. Application of the MS-compatible solubilizers of the invention as a matrix additive for MALDI-TOF-MS analysis reduces the complexity of sodium-rich peptide samples without affecting the sensitivity of the analysis.

### Analyzable Surface Area

Detection of high molecular weight proteins by MALDI-TOF-MS can be challenging due to their inherent poor ionization efficiency. In order to detect such high molecular weight analytes, higher laser intensities, longer acquisitions and more spectra are generally needed to sum and average raw data in order to maximize signal-to-noise.

These concerns can be met by, for example, increasing the analyzable surface area of a MALDI target; only selected positions (“sweet spots”) within the sample spot lead to useful spectra. The analyzable surface area can increased by optimizing sample and matrix preparation conditions, as well as sample and matrix spotting for MALDI analysis.

Limitations in the analyzable surface area and its homogeneity on a target surface also makes automation of MALDI difficult. Charles Cantor neatly summarized the problem: “There is a problem in that MALDI-MS is hard to automate. MALDI yields excellent data, but in most conventional MS one has to search around the sample to find what is called a ‘sweet spot.’ If one simply hits the sample with a laser at random, no useful data are obtained. A manual search has to be performed, usually under the trained eye of an experienced person looking for the one little place in the sample that gives good MS results.” See page 20 of: Serving Science and Society in the New Millenium: DOE's Biological and Environmental Research Program, U.S. Department of Energy, National Research Council, National Academy Press, Washington, D.C., 1998.

### Signal-to-Noise Ratio

In MALDI-MS, due to any of the above factors, acting alone or in combination with each other and/or other factors, the signal-to-noise ratio can be low and difficult to increase to an acceptable or preferable degree. The signal-to-noise ratio (a.k.a. SNR) is the ratio of the intensity of a signal (meaningful information) to the intensity of background “noise”. Data with higher SNR are desirable as they are “cleaner”, i.e., they have a higher density of information.

There have been attempts to use buffer salt additives such as ammonium citrate or ammonium phosphate to MALDI matrices in an attempt to increase signal-to-noise. For instance, MALDI-MS standards from Applied Biosystems recommend mixing into a matrix solution containing 50 mM ammonium phosphate or ammonium citrate.

MALDI-MS analysis of oligonucleotides can be carried using buffer additives such as tetraamine spermine (Asara et al., Anal. Chem. 71:2866, 1999), fucose (Distler et al., Anal. Chem. 73:5000, 2001) and other sugars (Shahgholil et al., Nucleic Acids Res. 29:e91, 2001). These additives however, result in marginal improvements in the reduction of background noise via matrix clusters, and are largely ineffective in the presence of high salt concentrations (>100 mM).

Another method of removing salt contaminants is to wash the spotted analyte:matrix mix with cold water. However, this method can lead to losses of small polar peptides, or peptides modified with polar moieties. Thus, it is generally not useful for quantitative studies intended to measure the efficiency of a post-translational modification, as one or more of the forms may be washed out.

### SUMMARY OF THE INVENTION

The invention provides compositions and methods useful in mass spectrometry (MS), including without limitation LC/MS and MALDI-TOF MS, often referred to herein simply as MALDI MS.

The invention provides reagents for use in preparing target molecules for mass spectrometry, in which the reagents are mass spectrometry compatible (“MS-compatible”), meaning that they do not reduce the quality of the mass spectra obtained when a target molecule is analyzed by MS. The invention further, provides reagents that improve the quality of mass spectra obtained by MS analysis, such as but not limited to MALDI MS.

In one aspect the invention provides MS-compatible solubilizers that can increase the solubility of an analyte. The MS-compatible solubilizers can include without limitation detergents or surfactants. Blends of solubilizers are included, in which the blends can include one or more detergents or one or more surfactants, and one one or more detergents in combination with one or more surfactants. The solubilizer blends can optionally further include buffers, chaotropic agents, salts, or other chemical entities. The solubilizers and solubilizer blends can be present during mass spectrometry analysis, such as by MALDI MS or LC/MS.

In another aspect the invention provides MS-compatible sorbents. The invention provides MALDI matrix additives that support and/or promote the formation of small analyte:matrix crystals in thin layers. Non-limiting examples of MS-compatible sorbents are silicas, aluminia, germanium oxide, indium tin oxide, metal oxides, chlorides, sulfates, phosphates, carbonates and fluorides; polymer based oxides, chlorides, sulfates, carbonates, phosphates or fluorides; diatomaceous earth; graphite or activated charcoal; and titania, gold and activated gold.

In yet another aspect the invention provides MS-compatible buffers. In some embodiments, the MS-compatible buffer is a morpholino-sulfonic acid, such as 2-(n-morpholino)ethane sulfonic acid (MES); 4-(n-morpholino)butane-sulfonic acid (MOBS); 3-(n-morpholino)propane-sulfonic acid (MOPS); or 3-(n-morpholino)-2-hydroxypropanesulfonic acid (MOPSO).

The invention also provides compositions and methods of preparing a hydrophobic molecule for MALDI-TOF MS analysis, the methods comprising contacting a composition comprising said hydrophobic molecule with at least one MS-compatible solubilizer.

The invention also provides compositions and methods for performing LC/MS analysis of a sample, the methods comprising contacting a sample with at least one MS-compatible solubilizer and performing LC/MS analysis of a sample. In one aspect, the invention provides methods of performing isolelectric focusing of a sample, where the sample has been contacted with at least one MS-compatible solubilizer. The sample or a portion thereof. can be analyzed by mass spectrometry after isoelectric focusing has been performed.

The invention also provides compositions and methods for preparing a protein for MALDI-TOF analysis, the method comprising contacting a composition comprising the protein, in any order or combination, with (a) at least one MALDI matrix additive of the invention and (b) at least one enzyme, such as a protease or a protein-modifying enzyme. By way of non-limiting example, in the case of peptide mass fingerprinting (PMF), the enzyme is a protease. In a more specific embodiment, the invention provides compositions and methods for preparing a protein having one or more hydrophobic regions for MALDI-TOF analysis, the method comprising contacting a composition comprising said protein, in any order or combination, with (a) at least one MS-compatible solubilizer and (b) at least one enzyme, such as a protease or a protein-modifying enzyme.

In another embodiment, the invention provides compositions and methods of preparing a sample comprising a protein, such methods comprising (a) subjecting a sample comprising the protein to a process that at least partially separates the protein from other molecules in the sample, to generate a partially purified protein, and (b) contacting a composition comprising the partially purified protein with MALDI matrix additive of the invention, thereby generating a sample comprising the protein suitable for MALDI-TOF analysis. An enzyme, such as a protease, and/or a matrix suitable for MALDI-TOF may also be added at (b) or at some other point in sample preparation. In instances wherein the sample comprises a protein having one or more hydrophobic regions for MALDI-TOF analysis, a preferred MALDI matrix additive is a MS-compatible solubilizer.

In another embodiment, the invention provides compositions and methods for identifying a region on a molecule that binds to a region on a ligand, comprising contacting the molecule and/or the ligand with at least one MALDI matrix additive of the invention, thereby generating a sample suitable for MALDI-TOF analysis, and subjecting the sample to MALDI-TOF analysis. The molecule and/or the ligand can be hydrophobic, or comprise at least one region that is hydrophobic, in which case a preferred MALDI matrix additive is a MS-compatible solubilizer of the invention.

In another embodiment, the invention provides compositions and methods for identifying a protein that binds to a ligand, the method comprising (a) contacting, in any order or combination, (i) a sample comprising one or more proteins, (ii) the ligand, (iii) one or more cross-linkers, (iv) a MALDI matrix additive of the invention, and (v) a protease; in order to generate cross-linked peptides, which are cross-linked to the ligand or some portion thereof, and determining the amino acid sequences of the cross-linked peptides by MALDI-MS analysis. The amino acid sequence of the cross-linked peptides comprise all or part of a region on a protein that binds to said ligand.

In another embodiment, the invention provides compositions and methods for identifying a protein or region thereof that is chemically modified, said method comprising: (a) contacting, in any order or combination, (i) the protein, (ii) an enzyme that modifies proteins, (iii) a MALDI matrix additive of the invention, and (iv) a protease, in order to generate chemically modified peptides. The amino acid sequences of the chemically modified peptides are determined by MALDI-TOF analysis; these amino acid sequences comprise all or part of a region on a protein that is chemically modified by the enzyme.

In another embodiment, the invention provides compositions and methods for extending sequence coverage in peptide-mass fingerprinting, comprising contacting the peptide with a MALDI matrix additive of the invention.

In another embodiment, the invention provides compositions and methods for inhibiting the formation of protein:ion adducts in a protein, comprising contacting the protein with a MALDI matrix additive of the invention.

In another embodiment, the invention provides compositions and methods for evaluating uncharacterized compounds and compositions for their potential as matrix additives of the invention (e.g., MS-compatible solubilizers, MS-compatible sorbents and/or MS-compatible buffers).

In further embodiments, the invention provides kits comprising one or more containers comprising at least one of the MALDI matrix additives of the invention. Such kits can further comprise one or more kit components. Illustrative examples of such kits are provided herein (see especially Examples 11, 12 and 22).

In one aspect, the invention is drawn to a solid support comprising or coated with a MS-compatible composition. The MS-compatible composition can be a MS-compatible solubilizer, or a composition comprising one or more MS-compatible non-volatile MALDI additives. The solid support can be in the form of a bead, a monolithic column or chip surface or the interior of chromatographic tubing. In some embodiments, the solid support is a MS target surface

In one embodiment, the invention is drawn to a method of coating a substrate, comprising contacting said substrate to one or more MS-compatible compositions. The MS-compatible composition can be a MS-compatible solubilizer, or a composition comprising one or more MS-compatible non-volatile MALDI additives. In some embodiments, such methods involve electrospraying.

Unless otherwise defined, all technical and scientific terms used herein have the meaning commonly understood by one skilled in the biotechnology art. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description.

### DESCRIPTION OF THE DRAWINGS

FIG. 1: SDS-PAGE of purified T. californica nAChR.

FIG. 2: MALDI-MS spectra for the peptide mass fingerprint of the nAChR delta (A), gamma (B), beta (C) and alpha (D) subunits.

FIG. 3: Amino acid sequences of the nAChR delta (A), gamma (B), beta (C) and alpha (D) subunits. Bold text signifies residues identified by MALD-TOF as described in the Examples; underlined text corresponds to transmembrane domains.

FIG. 4: MALDI-TOF-MS analysis of the PMF for BSA. The spectra of trypsinized BSA without (A) or with (B) 1× IMB between m/z 825 and 1700 are shown. The numbers above each mass-ion identify the corresponding region of the amino acid sequence of BSA. The font color is coordinated with identified NA⁺ adducts. The spectra on the right are expanded views of the spectra between m/z 1190 and 1310.

FIG. 5: MALDI-TOF-MS analysis of Bradykinin. Bradykinin (m/z 998.58) was analyzed without IMB in the presence of (A) 50 mM NaCl or (C) 50 mM KCl; the sodium adducts (m/z 1020.56, 1042.54) and potassium adduct (m/z 1036.55) are identified by green font. When 0.4× IMB 1:1 (v/v) was included in these experiments, in either (B) 50 mM NaCl or (D) 50 mM KCl, the signal from the adducts was reduced or eliminated.

FIG. 6: Plot of normalized intensity of +Na⁺ adduct versus concentration of IMB.

FIG. 7: Plot of normalized intensity of +Na⁺ adduct versus laser intensity.

FIG. 8: MALDI-MS spectra of 100 fmol of BSA in (A) 50% acetonitrile/0.1%TFA (trifluoroacetic acid) and (B) in 1× BLEND 1/0.7 M urea/0.7 M thiourea.

FIG. 9: Chromatographic separation of cytochrome P450 tryptic digest in the presence of Blend II monitored as a total ion count by ESI-MS.

FIG. 10: SDS-PAGE of acetone-preciptated proteins (myoglobin, BSA and nAChR) resuspended in distilled water (dH₂O), NuPAGE buffer, or BLEND II with 4 M urea.

FIG. 11: Recovery of nAChR from dialysis using BLEND II.

FIG. 12: Total ion chromatograph for a cytochrome P450 (SEQ ID NO:8) 2D6 digest in BLEND II.

FIG. 13: In-gel digestion of nAChR in the presenced or absence of BLEND II.

FIG. 14: Spectra of mass standards in ordinary MALDI matrix versus matrix with silica additive. The top two spectra show MALDI-MS analyses of a calibrant mixture in (A) conventional alpha-cyano (CHCA) and (B) alpha-cyano:silica mixture. The bottom two spectra show MALDI-MS analyses of a calibrant mixture in the presence of 500 mM NaCl in (C) conventional alpha-cyano and (D) alpha-cyano:silica mixture.

FIG. 15: Spectra of a tryptic digest of Ovalbumin (1 pmol) analyzed by MALDI-MS using (A) a conventional CHCA (αC) matrix and (B) Maxlon AC, in which silica is present.

FIG. 16: MALDI-MS analysis of tryptic digest of beta-galactosidase in (A) conventional CHCA (alpha-cyano) and (B) Maxlon AC, in which silica is present.

FIG. 17: Spectra of tryptic digests of 100 fmol of beta-galactosidase in 500 mM NaCl in (A) conventional CHCA (αC) matrix or (B) MaxIon AC, in which silica is present.

FIG. 18: Images of 1 μL spots of SA dissolved in the absence or presence of MES, prepared and stored as described (in brief, A=freshly prepared sinapinic acid (SA); B=SA prepared as in A but stored 8 months at 8° C.; C=Maxlon SA matrix stored at 8° C. for 8 months), and after the number of laser shots indicated on the left (1=200 laser shots; 2=10,000 laser shots; and 3=20,000 laser shots).

FIG. 19: MALDI MS spectra of intact proteins (insulin, ubiquitin and cytochrome-c) co-spotted with SA only (A1-A3, B1-B3) or co-spotted with SA/MES (Maxlon SA) (C1-C3). (A, B, C, 1, 2 and 3 are as described for FIG. 13).

FIG. 20: Analysis of a HMW standard (159,081 Da) using (A) sinapinic acid dissolved in 0.1% TFA/50% ACN and (B) sinapinic acid dissolved in MaxIon SA.

FIG. 21: Chemical structures of (A) sinapinic acid and MES and (B) MES, MOPS, MOPSO and MOBS.

FIG. 22: The silica resin of MaxIon AC tested under different storage conditions and analyzed by MALDI-MS of InvitroMass LMW Cal 2 (1:200 in 100 mM NaCl).

FIG. 23: Matrix solution (0.5 μL) was co-spotted with 0.5 μL of 100 mM NaCl solution onto a stainless steel MALDI target plate (A) without or (B) silica.

FIG. 24: Spectra resulting from MALDI analysis of the target surfaces shown in FIG. 18 gathered by random scanning of (A) the plate with silica and (B) the plate lacking silica.

### DEFINITIONS

In the description that follows, a number of terms used in recombinant nucleic acid technology are utilized extensively. In order to provide a clear and more consistent understanding of the specification and claims, including the scope to be given such terms, the following definitions are provided.

Analyte: The terms “analyte” and “molecule of interest” are used interchangeably herein to indicate a molecule that one wishes to detect, quantify or otherwise examine or study. That is, the use of the term term “analyte” herein is not limiting to only determining the type or amount of a molecule of interest; rather, it encompasses other observations regarding e.g., ligand:ligand interactions and conformational change of molecules.

Bead: A spheroidal solid support. A bead can but need not be hollow, or can comprise openings from the outer surface of the bead that lead to one or more internal surfaces. The external and/or internal surfaces of a bead can be coated with a molecule having one or more useful properties. For example, a bead coated with a binding moiety (e.g., an antibody) can be contacted with a sample that contains the ligand (e.g., the antigenic target of the antibody), and the bead will bind and retain the ligand (antigen). Often, after the ligand has been adsorbed by the bead, the bead is washed or otherwise treated to remove undesirable contaminants, and the ligand, a molecule of interest, is eluted from the bead. In some applications, a population of beads is placed in a hollow container within which flows a fluid containing a molecule of interest, and the beads' surfaces are coated with a binding moiety, or an enzyme. For example, a molecule of interest, which is a substrate for a given enzyme, is contacted with beads coated with that enzyme, and the products of the chemical (enzymatic) reaction are generated. The products might remain in solution, or one or more desirable products might remain bound to the bead and separately eluted later, or a desirable product might be released into solution while an undesirable product remains bound to the bead. A sample can be contacted with a population of beads by fluid passage through a bead-filled container (e.g., a column) followed by optional washes and elution, or by preparing a mixture of beads and sample, which is then centrifuged to make the beads form a pellet, followed by optional washes and elution.

Biomolecule: The term “biomolecule” encompasses any molecule produced by a living organism or a fragment thereof. The term “biopolymer” as used herein means any polymeric molecule produced by a living organism or a fragment thereof. Either type of molecule can be a polypeptide, a protein, a nucleic acid, a polynucleotide, a carbohydrate, a lipid, a polysaccharide, or a fragment or derivative thereof.

Chaotrope: The term “chaotrope” as used herein refers to a chemical agent that denatures proteins. Exemplary chaotropes include urea, thiourea and guanidine hydrochloride. The terms “chaotrope”, “denaturing agent”, and “denaturant” are used interchangeably herein.

Coat: As used herein, the term “coat” refers to a layer of a substance on the surface of a solid support, which can be, for example, a bead; all or a portion of a well in a microtiter plate; the inner surface of a container; and the like.

Colloid: As used herein, a “colloid” is a mixture composed of particles (the dispersed phase) suspended in a medium (a continuous mobile phase), having properties between those of a solution and a fine suspension. Colloidal particles generally have at least one dimension in the range of from 1 (or about 1) nm to 100 (or about 100) micrometers, particularly from 10 (or about 10) nm to 50 (or about 50) micrometers, particularly from 10 (or about 10) nm to 10, 15 or 20 (or about 10, 15 or 20) micrometers. Unless otherwise specified, as used herein “colloid” refers to the colloid mixture per se and the particles without the mobile phase (i.e., dried particles). The term “colloid” also encompasses sols, slurries, colloidal suspensions and resins.

Colloidal suspension: As used herein, a “colloidal suspension” (a.k.a. colloidal solution) is a thermodynamically stable colloid comprised of particles suspended in a liquid. Typically, a colloidal suspension can be observed to have the Tyndall Effect, in which the reflection of a light beam passing through a colloid identifies the presence of suspended particles.

Cross-linker: The terms “cross-linker” and “cross-linking agent” are used interchangeably herein and are intended to refer to a typically bifunctional (two-armed) chemical linker that can be added to a mixture of molecules to form covalent linkages between two or more molecules. Such bifunctional cross-linkers can be homobifunctional (wherein both “arms” of the linker are the same chemical moiety) or heterobifunctional (wherein each of the two “arms” is a different chemical moiety than the other). Reactive groups that can be targeted using a cross-linker include primary amines, sulfhydryls, carbonyls, carbohydrates and carboxylic acids. Many cross-linkers are described and made commercially available by Pierce Biotechnology, Inc. (Rockford, Ill.).

Detectably labeled: The terms “detectably labeled” and “labeled” are used interchangeably herein and are intended to refer to situations in which a molecule (e.g., a nucleic acid molecule, protein, nucleotide, amino acid, and the like) have been tagged with another moiety or molecule that produces a signal capable of being detected by any number of detection means, such as by instrumentation, eye, photography, radiography, and the like. In such situations, molecules can be tagged (or “labeled”) with the molecule or moiety producing the signal (the “label” or “detectable label”) by any number of art-known methods, including covalent or ionic coupling, aggregation, affinity coupling (including, e.g., using primary and/or secondary antibodies, either or both of which may comprise a detectable label), and the like. Suitable detectable labels for use in preparing labeled or detectably labeled molecules in accordance with the invention include, for example, radioactive isotope labels, fluorescent labels, chemiluminescent labels, bioluminescent labels and enzyme labels, and others that will be familiar to those of ordinary skill in the art.

Domain: The terms “domain” and “protein domain” are used interchangeably herein to refer to a relatively small (i.e., <about 150 amino acids) globular unit that is part of a protein. A protein may comprise two or more domains that are linked by relatively flexible stretches of amino acids. In addition to having a semi-independent structure, a given domain may be largely or wholly responsible for carrying out functions that are normally carried out by the intact protein. In addition to domains that have been determined by in vitro manipulations of protein molecules, it is understood in the art that a “domain” may also have been identified in silico, i.e, by software designed to analyze the amino acid sequences encoded by a nucleic acid in order to predict the limits of domains. The latter type of domain is more accurately called a “predicted” or “putative” domain but, in the present disclosure, the term domain encompasses both known and predicted domains unless stated otherwise.

Hydrophilic: The terms “hydrophilic” and “lipophobic” are used interchangeably herein and refer to compounds and substances that tend to dissolve in, mix with or be wetted by, water. Hydrophilic or lipophobic species, or hydrophiles, tend to be electrically charged and polar, and thus preferring other charged and polar solvents or molecular environments. Non-limiting examples of hydrophilic molecules include lipids and hydrophilic proteins.

Hydrophobic: The terms “hydrophobic” and “lipophilic” are used interchangeably herein and refer to compounds and substances that tend to not dissolve in, mix with or be wetted by, water. Hydrophobic or lipophilic species, or hydrophobes, tend to be electrically neutral and nonpolar, and thus preferring other neutral and nonpolar solvents or molecular environments. Non-limiting examples of hydrophobic molecules include alkanes, oils, fats, lipids and hydrophobic proteins.

Ligand: The term “ligand” as used herein refers to a small molecule that binds to a larger macromolecule. Examples of ligands are a synthetic compound, such as a drug or drug candidate (lead compound), or a biomolecule, e.g., antibody, an agonist, an antagonist, an allosteric modulator, a phospholipid, cholesterol, a fatty acid, a steroid, a hormone, a volatile anesthetic, a fluxing ion, an ion cofactor or modulator, or combinations thereof.

Molecule: The term “molecule” has its normal scientific meaning herein, but also includes molecular complexes.

Molecular Complex: A type of molecule (as that term is used herein) that consists of two or more molecules that are at least partially bound to each other due to non-chemical interactions. Examples of some molecular complexes of particular interest include protein:nucleic acid complexes, e.g., recombination complexes, topoisomerase complexes, RNA or DNA polymerase holoenzymes, ribosomes, and the like.

Monomer: The term “monomer” as used herein refers to the unimolecular form of a molecule that can achieve a multimeric form.

Multimer: The term “multimer” as used herein refers to a complex or compound formed by the assembly of 2 or more monomers. One example of a multimer is a protein complex that is formed of two or more copies of the same polypeptide, e.g., the homodimeric nucleoid-associated protein HBsu of Bacillus subtilis. Another example is a lipids that can assemble into aggregate structures such as micelles and bilayers.

Non-volatile: As used herein, the phrase “non-volatile” refers to a composition that is not readily vaporizable at a relatively low temperature, especially room temperature.

Nucleic Acid Molecule: As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

Of interest: As used herein, the term “of interest” is used to indicate a particular object or process that one wishes to detect, identify, quantify, determine or monitor the activity or properties of, and/or otherwise observe. Unless otherwise indicated, as used herein the term “molecule of interest” is synonymous with “analyte”.

Polypeptide: As used herein, the term “polypeptide” refers to a sequence of contiguous amino acids of any length, i.e., a linear molecule composed of two or more amino acids linked by covalent (peptide) bonds. The terms “peptide,” “oligopeptide,” or “protein” may be used interchangeably herein with the term “polypeptide.” The term “protein” includes polypeptides as well as protein complexes formed of 2 or more polypeptides.

Purified: The term “purified” refers to a compound that has been separated from at least 50% or about 50% of undesirable elements in a mixture containing the compound. As used herein the term “substantially purified” means at least 95% or about 95%, preferably at least 99% or about 99%, free of other components in a starting mixture.

Resin: As used herein, the term “resin” refers to the polymeric base (which may be chemically modified, e.g., cross-linked to one or more other substances) of some ion-exchange materials used in chromatography. The polymeric base may be, but need not be, polystyrene.

Sample: As used herein, the term “sample” refers to any composition that is subject to analysis. Typically, a sample comprises, or is suspected of comprising, an analyte of interest.

Separated: The term “separated” as used herein refers to a compound that has been physically separated from at least one other element in a mixture containing the compound.

Slurry: The term “slurry” refers to a thin mixture of a liquid, especially water, and any of several finely divided substances, such as beads, cement, plaster of Paris, or clay particles.

Solid support: As used herein, the term “solid support” means a non-gaseous, non-liquid, solid or semi-solid material having a surface. Thus, a solid support can be a flat surface constructed, for example, of glass, silicon, metal, plastic or a composite; or can be in the form of a bead such as a silica gel, a controlled pore glass, a magnetic or cellulose bead; or can be a pin, a monolithic column, a chip surface, or the interior of chromatographic tubing including an array of pins suitable for combinatorial synthesis or analysis.

Solubilizer: The terms “solubilizer” and “solubilizing agent” are used interchangeably herein and refer to any compound or mixture of compounds that enhances the solubility of a hydrophobic compound.

Solution: As used herein, a “solution” is a homogeneous mixture that is a single-phase mixture composed of a solute and a solvent. The dissolved particles (solutes) are small molecules and ions between 1 Å and 100 Å in diameter.

Surfactants: Surface active molecules or compositions; also known as wetting agents. Surfactants are used to provide detergency and emulsification. The term “surfactant” encompasses detergents as well as non-detergents (e.g., non-detegent sulfobetaines, a.k.a. NDSBs).

Suspension: As used herein, a “suspension” is a two-phase mixture composed of a dispersed and continuous phase. The particles of the dispersed phase are generally larger than about 10,000 Å to about 100,000 Å (i.e., from about 1 micrometer to about 10 micrometers) thick in at least one dimension (e.g., length, width, height, depth diameter.

Other terms used in the fields of recombinant nucleic acid technology and molecular and cell biology as used herein will be generally understood by one of ordinary skill in the applicable arts.

### DETAILED DESCRIPTION OF THE INVENTION

I. MS-Compatible Reagents

The invention is drawn to reagents that can be used in MS that improve the solubility of analytes during sample processing (for any type of MS analysis) or in MALDI sample-matrix formulations, increase the signal-to-noise ratio of mass spectra, reduce the size of adduct cluster peaks in mass spectra, increase the analyzable surface area of a MALDI sample, or improve the stability of an analyte:matrix crystal used in MALDI-MS.

In one aspect, the invention is drawn to MALDI matrix additives. Such MALDI matrix additives comprise one or more substances selected from the group consisting of a MS-compatible solubilizer, a MS-compatible sorbent, and a MS-compatible buffer. However, the invention is not limited in use to MALDI MS or with MALDI matrices. For example, the solubilizers, buffers, and reagents used in the invention can find use in other types of MS analysis, including LC/MS.

In various aspects, an MS-compatible reagent of the invention comprises one or more MS-compatible solubilizers, one or more MS-compatible sorbents, and/or one or more MS-compatible buffers. In other aspects, the invention is drawn to compositions and kits comprising one or more MS reagents of the invention, and methods of making and using such compositions and kits.

MALDI-TOF MS is generally used to study specified or preselected MS target molecules. The term “MS target molecule” of “analyte” refers to a molecule of interest that is being studied using MS. Non-limiting examples of MS target molecules include peptides; proteins; protein:protein complexes; protein:DNA complexes; oligonucleotides; nucleic acids, such as DNA and RNA; nucleic acid:nucleic acid complexes; oligosaccharides; lipids, including phospholipids; synthetic polymers; small organic molecules; and complexes of any of the above. Any of these molecules can be from a biological source (“biomolecules”) or from in vitro chemical synthesis (“synthetic molecules”). In some embodiments, the molecule is a hydrophobic molecule, such as a lipid or a hydrophobic protein (e.g., a membrane protein), but the invention is applicable to hydrophilic molecules (e.g., soluble proteins) as well.

MS reagents of the invention are not necessarily present in the analyte:matrix crystals placed on a MALDI target surface. They are, however, present for at least part of the MALDI analysis procedure and can thus be added at one or more various points, including without limitation: during sample preparation or sample processing procedures; during preperation of the matrix molecules; during formation of analyte:matrix crystals; during washing of the target surface; etc. The term “sample processing procedures” refers to procedures involving preparing a sample for MALDI-TOF MS, including without limitation (a) analyte isolation; (b) sample processing; (c) mixing of the analyte, matrix and optional additives; (d) co-precipitation of analyte:matrix to produce analyte:matrix crystals; (e) deposition of analyte:matrix crystal onto a MS target surface; and (f) washing analyte:matrix crystals in situ.

Because they are not necessarily present in the analyte:matrix crystals placed on a MALDI target surface, the MS-compatible reagents of the invention can be partially or totally removed at any time after their addition. Moreover, the additives can be combined with any other component(s) in any fashion and in any appropriate order.

In many embodiments, the MS-compatible reagents provided herein are used as matrix additives that are present in a MALDI matrix crystal during MS analysis. By way of non-limiting example, the additive can be combined with a matrix and a sample comprising an analyte as those two componenents are mixed, or “pre-mixed” with matrix or analyte before matrix and analyte are combined. In the latter instance, for example, matrix molecules can be dissolved in a diluent that comprises one or more matrix additives of the invention. Moreover, when a matrix additive of the invention is present in analyte:matrix crystals, crystals comprising the additives are also part of the claimed invention.

The matrix additive can be a MS-compatible solubilizer, such as one or more sulfobetaines, one or more non-detergent sulfo-betaines, a MS-compatible sorbent, such as silica, and/or a MS-compatible buffer of the invention.

The compositions and methods of the invention can be applied to any appropriate analyte. The analyte can be a hydrophilic molecule or a hydrophobic molecule. The analyte can be a protein complex, or a molecule selected from the group consisting of other proteins, peptides, DNA, RNA, oligonucleotides, nucleic acids, oligosaccharides, polysaccharides, lipids, phospholipids, synthetic polymers, small organic molecules, and complexes or combinations of any of the above.

### MS-Compatible Solubilizers

In one aspect, the invention provides compositions comprising solubilizing agents (solubilizers) that are not, unlike other solubilizers, largely incompatible with mass spectrometry (i.e., they are MS-compatible); MS-compatible matrix additives; mixtures, including crystals, comprising MS matrix and/or analyte molecules and one or more MS-compatible compositions (e.g., a MS-compatible solubilizer or a MS-compatible matrix additive); and solutions comprising one or more MS-compatible compositions. In many embodiments, the MS-compatible solubilizer is not removed from a sample or analyte during MS, but is present during mass spectrometry. The provided MS-compatible solubilizers do not have deleterious effects on mass spectra of analytes.

The MS-compatible solubilizers can include without limitation detergents or surfactants. Blends of solubilizers are included, in which the blends can include one or more detergents or one or more surfactants, and one one or more detergents in combination with one or more surfactants. The solubilizer blends can optionally further include buffers, chaotropic agents, salts, or other chemical entities.

A MS-compatible solubilizer of the invention can be a MS-compatible detergent, a MS-compatible non-detergent, or combinations thereof. More specifically, a MS-compatible solubilizer of the invention comprises one or more components selected from the group consisting of (a) one or more MS-compatible detergents, wherein at least one of the detergents is at a concentration that is at least about 5% of its CMC when in solution with the analyte prior to crystal formation, more preferably at a concentration that at least about 75% of its CMC when in solution with the analyte prior to crystal formation; and (b) one or more MS-compatible non-detergent surfactants, wherein an effective amount of said MS-compatible solubilizer has one or more of the following characteristics when used in mass spectrometry studies: (i) it improves the solubility of an analyte by at least about 5% during one or more sample processing procedures, (ii) it improves the solubility of an analyte by at least about 5% in a composition comprising a matrix, (iii) it improves the stability of an analyte:matrix crystal by at least about 5%, (iv) it increases the analyzable surface area of an analyte-matrix crystal by at least about 1%, (v) it increases the signal-to-noise ratio by at least about 5%, and/or (vi) it diminishes by at least about 5% one or more adduct cluster peaks of a molecule that forms adduct with ions.

In some preferred embodiments, MS-compatible solubilizing compositions include at least one detergent that is present at a concentration close to its CMC. In some preferred embodiments, MS-compatible solubilizing compositions include at least one detergent that is present at a concentration that is at or above its CMC.

The compositions can included two or more MS-compatible detergents, each of which is at a concentration of at least about 75% of its CMC when in solution with an analyte prior to mass spectrometry. The compositions can included two or more MS-compatible detergents, each of which is at a concentration of close to its CMC when in solution with an analyte prior to mass spectrometry. The compositions can included two or more MS-compatible detergents, each of which is at a concentration at or above its CMC when in solution with an analyte prior to mass spectrometry.

Typically, analytes are prepared for MS-MALDI analysis by contacting a sample comprising analyte molecules with molecules of a MALDI matrix material. The analyte and matrix molecules co-precipitate to form what is called an “analyte:matrix crystal” herein. The crystal, which is formed on a MALDI target surface, is subject to pulses of laser irradiation and, as a result, the analyte molecules are ionized. The ions are subject to further analysis, e.g., time-of-flight (TOF) analysis.

In some embodiments, an effective amount of a MS-compatible solubilizer of an invention increases the signal-to-noise ratio from at least about 5% to about 100-fold. Typically, for the present invention, the MS-compatible solubilizer is used at least at a concentration at which it is effective to improve the solubility of the molecule of interest by about 10% during analyte:matrix crystallization and/or during laser exposure in MALDI-MS, preferably resulting in an at least about 10% increase of signal-to-noise ratio.

An MS-compatible solubilizer can also be used to enhance solubility of one or more analytes using other types of MS, in particular LC/MS. For example, an MS-compatible solubilizer can allow for better yield and purification of proteins separated by, for example, HPLC, RP HPLC, capillary electrophoresis, or liquid or gel phase isolelectric focusing prior to MS analysis.

### Detergents and Other Surfactants in Mass Spectrometry

A MS-compatible solubilizer of the invention comprises one or more surfactants. A surfactant may be a detergent or a non-detergent surfactant, or combinations thereof. A solubilizer that is a mixture (blend) of surfactants can be MS-compatible even if individual surfactants are not. In some embodiments, however, MS-compatible surfactants are preferred.

### Non-Detergent Surfactants

Non-limiting examples of non-detergent surfactants include the non-detergent sulfobetaines (NDSBs). The NDSBs are zwitterionic compounds that have a sulfobetaine hydrophilic group and a short hydrophobic group. They cannot aggregate to form micelles, and NDSBs are thus not considered detergents. NDSBs that can be used in the invention include without limitation those listed in Table 1. In some preferred aspects of the invention, a solubilizer composition of the invention comprises at least one NDSB. The concentration of an NDSB in a solubilizer composition can vary, for example such that the concentration of the NDSB when it contacts a sample or analyte is from about 10 micromolar to about 2M, such as from about 100 micromolar to about 1M, or from about 1 mM to about 800 M. In preferred embodiments, the concentration of an NDSB when it contacts a sample or analyte is at least about 5 mM, such as from about 10 mM to about 600 mM, or from about 20 micromolar to about 400 mM, or from about 50 mM to about 300 mM.

TABLE 1
NON-LIMITING EXAMPLES OF NON-DETERGENT
SULFOBETAINES (NDSBs)

| | |
|---|---|
| NDSB-195 | Dimethylethylammonium-1-propanesulfonate |
| NDSB-201 | 3-(1-Pyridino)-1-propanesulfonate |
| NDSB-211 | Dimethyl-2-hydroxyethyl-1-propanesulfonate |
| NDSB-221 | 3-(1-Methylpiperidinium)-1-propanesulfonate |
| NDSB-223 | N-Methyl-N-(3-sulfopropyl)morpholinium |
| NDSB-256 | Dimethylbenzylammonium-1-propanesulfonate |

### Detergents

A detergent is a compound, or a mixture of compounds, the molecules of which have two distinct regions: one that is hydrophilic, and readily dissolves in water, and another that is hydrophobic, with little (if any) affinity for water. A detergent is thus an amphipathic surface-active molecule, i.e., one type of surfactant. Unlike non-detergent surfactants, detergents can form micelles.

Detergents can be described as falling into one of three groups: ionic, non-ionic and zwitterionic. Non-ionic detergents are molecules that do not ionize in aqueous solutions. Ionic detergents can be divided into those having cationic (positively charged) and anionic (negatively charged) detergents. A Zwitterion (German for “hybrid ion”) is a neutral compound having electrical charges of opposite sign, delocalized or not on adjacent or nonadjacent atoms. Zwitterionic compounds have no uncharged canonical representations, and can behave like an acid or a base, depending on conditions.

Preferred detergents for use in MS-compatible solubilizer compositions include nonionic detergents and zwitterionic detergents. In illustrative embodiments, nonionic detergents used in MS compatible solubilizer compositions can be glycopyranosides, and include but are not limited to nonionic detergents having glucose, maltose, or sucrose moieties. For example, n-ethyl-beta-D-glucopyranoside, n-propyl-beta-D-glucopyranoside, n-tetryl-beta-D-glucopyranoside, n-pentyl-beta-D-glucopyranoside, n-hexyl-beta-D-glucopyranoside, n-heptyl-beta-D-glucopyranoside, n-octyl-beta-D-glucopyranoside, octyl-beta-D-1-thioglucopyranoside, n-nonyl-beta-D-glucopyranoside, n-ethyl-beta-D-maltoside, n-propyl-beta-D-maltoside, n-tetryl-beta-D-maltoside, n-pentyl-beta-D-maltoside, n-hexyl-beta-D-maltoside, n-heptyl-beta-D-maltoside, n-octyl-beta-D-maltoside, n-nonyl-beta-D-maltoside, n-decyl-beta-D-maltoside, n-monodecyl-beta-D-maltoside, n-dodecyl-beta-D-maltoside, or n-dodeconoylsucrose.

Preferred zwitterionic detergents for use in MS compatible solubilizer compositions are sulfobetaine detergents, for example, SB or ASB detergents (e.g., SB-8, SB-10, SB-12, SB-14, SB-16, ABS-C80). The concentration of the detergents used can vary. Methods are provided herein for testing the effectiveness of detergent formulations in improving spectra. In preferred embodiments, at least one of the detergents used in a solubilizer for MS is contacted with a sample or analyte near or above its CMC.

### Anionic Detergents Include Without Limitation:

Glycochenodeoxycholic acid sodium salt; Glycocholic acid hydrate, synthetic; Glycocholic acid sodium salt hydrate; Glycodeoxycholic acid monohydrate; Glycodeoxycholic acid sodium salt; Glycolithocholic acid 3-sulfate disodium salt; and Glycolithocholic acid ethyl ester;

Sodium 1-butanesulfonate; Sodium 1-decanesulfonate; Sodium 1-dodecanesulfonate; Sodium 1-heptanesulfonate; Sodium 1-nonanesulfonate; Sodium 1-propanesulfonate monohydrate; and Sodium 2-bromoethanesulfonate;

Sodium cholate hydrate; Sodium choleate; Sodium deoxycholate; Sodium dodecyl sulfate; Sodium hexanesulfonate; Sodium octyl sulfate; Sodium pentanesulfonate; and Sodium taurocholate;

Taurochenodeoxycholic acid sodium salt; Taurodeoxycholic acid sodium salt monohydrate; Taurohyodeoxycholic acid sodium salt hydrate; Taurolithocholic acid 3-sulfate disodium salt; and Tauroursodeoxycholic acid sodium salt;

As well as Chenodeoxycholic acid; Cholic acid, ox or sheep bile; Dehydrocholic acid; Deoxycholic acid methyl ester; Digitonin; Digitoxigenin; N,N-Dimethyldodecylamine N-oxide; Docusate sodium salt; N-Lauroylsarcosine sodium salt; Lithium dodecyl sulfate; Niaproof 4, Type 4; 1-Octanesulfonic acid sodium salt; Trizma® dodecyl sulfate; and Ursodeoxycholic acid.

Cationic Detergents Include Without Limitation:

Alkyltrimethylammonium bromide; Benzalkonium chloride; Benzyldimethylhexadecylammonium chloride; Benzyldimethyltetradecylammonium chloride; Benzyldodecyldimethylammonium bromide; and Benzyltrimethylammonium tetrachloroiodate; Dimethyldioctadecylammonium bromide; Dodecylethyldimethylammonium bromide; Dodecyltrimethylammonium bromide; Ethylhexadecyldimethylammonium bromide; Hexadecyltrimethylammonium bromide; Thonzonium bromide; and Trimethyl(tetradecyl)ammonium bromide.

### Non-Ionic Detergents Include Without Limitation:

Span® detergents, including without limitation Span® 20; Span® 40; Span® 60; Span® 65; Span® 80; and Span® 85;

Tergitol detergents, including without limitation Tergitol, Type 15-S-12; Tergitol, Type 15-S-30; Tergitol, Type 15-S-5; Tergitol, Type 15-S-7; Tergitol, Type 15-S-9; Tergitol,

Type NP-10; Tergitol, Type NP-4; Tergitol, Type NP-40; Tergitol, Type NP-7; Tergitol, Type NP-9; Tergitol, Type TMN-10; and Tergitol, Type TMN-6;

Mega detergents, including without limitation Mega-8 and Mega-10;

N-Decanoyl-N-methylglucamine; n-Decyl a-D-glucopyranoside; Decyl beta-D-maltopyranoside; n-Dodecanoyl-N-methylglucamide; n-Dodecyl a-D-maltoside; n-Dodecyl-beta-D-maltoside; and n-Hexadecyl-beta-D-maltoside, n-dodeconoylsucrose;

Heptaethylene glycol monodecyl ether; Heptaethylene glycol monododecyl ether; and Heptaethylene glycol monotetradecyl ether;

Hexaethylene glycol monododecyl ether; Hexaethylene glycol monohexadecyl ether; Hexaethylene glycol monooctadecyl ether; and Hexaethylene glycol monotetradecyl ether;

Octaethylene glycol monodecyl ether; Octaethylene glycol monododecyl ether; Octaethylene glycol monohexadecyl ether; Octaethylene glycol monooctadecyl ether; and Octaethylene glycol monotetradecyl ether; Octyl-b-D-glucopyranoside; octyl-beta-D-1-thioglucopyranoside;

Pentaethylene glycol monodecyl ether; Pentaethylene glycol monododecyl ether; Pentaethylene glycol monohexadecyl ether; Pentaethylene glycol monohexyl ether; Pentaethylene glycol monooctadecyl ether; and Pentaethylene glycol monooctyl ether;

Polyethylene glycol diglycidyl ether; and Polyethylene glycol ether W-1;

Polyoxyethylene 10 tridecyl ether; Polyoxyethylene 100 stearate; Polyoxyethylene 20 isohexadecyl ether; and Polyoxyethylene 20 oleyl ether;

Polyoxyethylene 40 stearate; Polyoxyethylene 50 stearate; Polyoxyethylene 8 stearate; Polyoxyethylene bis(imidazolyl carbonyl); and Polyoxyethylene 25;

Tetraethylene glycol monodecyl ether; Tetraethylene glycol monododecyl ether; and Tetraethylene glycol monotetradecyl ether;

Triethylene glycol monodecyl ether; Triethylene glycol monododecyl ether; Triethylene glycol monohexadecyl ether; Triethylene glycol monooctyl ether; and Triethylene glycol monotetradecyl ether;

As well as APO-10; APO-12; Bis(polyethylene glycol bis[imidazoyl carbonyl]); Cremophor® EL; Decaethylene glycol monododecyl ether; Tyloxapol; and n-Undecyl-beta-D-glucopyranoside; Igepal CA-630; Methyl-6-O-(N-heptylcarbamoyl)-a-D-glucopyranoside; Nonaethylene glycol monododecyl ether; N-Nonanoyl-N-methylglucamine; NP-40; propylene glycol stearate; Saponins, e.g., Saponin from Quillaja bark; and Tetradecyl-b-D-maltoside.

### Zwitterionic Detergents Include Without Limitation:

Zwittergent® detergents, including without limitation Zwittergent® 3-08 (n-Octyl-N,N-dimethyl-3-ammonio-1-propanesulfonate); Zwittergent® 3-10 (n-Decyl-N,N-dimethyl-3-ammonio-1-propanesulfonate); Zwittergent® 3-12 (3-Dodecyl-dimethylammonio-propane-1-sulfonate); Zwittergent® 3-14 (n-Tetradecyl-N,N-dimethyl-3-ammonio-1-propanesulfonate); and Zwittergent® 3-16 (n-Hexadecyl-N,N-dimethyl-3-ammonio-1-propanesulfonate) [all of these Zwittergents are commercially available from EMD Biochemicals/Calbiochem, San Diego, Calif.];

3-(Decyldimethylammonio)propanesulfonate inner salt; 3-(Dodecyldimethylammonio)propanesulfonate inner salt; 3-(N,N-Dimethylmyristylammonio)propanesulfonate; 3-(N,N-Dimethyloctadecylammonio)propanesulfonate; and 3-(N,N-Dimethylpalmitylammonio)propanesulfonate;

Sulfobetaine detergents, including sulfobetaine SB8, sulfobetaine SB10, sulfobetaine SB12, sulfobetaine SB14, sulfobetaine SB16, and 4-n-Octylbenzoylamido-propyl-dimethylammoniosulfobetaine (ASB-C80);

as well as DDMAU; Lauryldimethylamine oxide (LADAO, LDAO); and N-Dodecyl-N,N-dimethylglycine.

Preferred MS-compatible detergents and surfactants include alkyl glycosides, sulfobetaines, non-detergent sulfobetaines, and bile acids.

Preferred detergents for use in the invention are MS-compatible meaning, in general, that they have no characteristics that interfere with a MALDI-TOF MS analysis of choice. An “MS-compatible” compound meets these characteristics: (1) it does not significantly interfere with the ionization efficiency of the analyte; (2) it does not form adducts to the protein or peptide that would interfere with mass determination; (3) it does not interfere w/ matrix crystal formation; and (4) it does not interfere with sample preparation. Detergents that can act as solubilizers for hydrophobic proteins are preferred.

CMC's for detergents can be found in CRC Guide for Surfactants and Lipids

Provided herein is a method for determining whether a compound such as a micelle-forming detergent or a surfactant-like compound is mass-spectroscopy compatible. This method provides another embodiment of the present invention. The method is illustrated in Example 1, herein.

### Micelles

An important characteristic of detergents and surfactants useful for practicing the present invention is the amount and type of aggregate structures present or formed during methods of MS and/or preparing samples for MS. In particular, monomers are preferred and aggregate structures, include without limitation liposomes and micelles, are less desirable. Detergent monomers assemble into aggregates called micelles, wherein the hydrophobic and hydrophilic moieties are exposed to the micelle interior and the aqueous environment, respectively.

In the case of individual detergents, known values for a compound's critical micelle concentration (CMC) can be used to predict conditions that favor the presence of monomers over aggregates. The CMC is the concentration of any given detergent that corresponds to the maximum possible concentration of detergent monomer in solution. Above the CMC, only the number of micelles increases with increasing concentration of detergent. Lowering the concentration of detergent below its CMC thus results in more monomers and fewer micelles. Micelles have a defined size and aggregate number (number of monomers in a micelle).

Methods and compositions of the present invention, in certain illustrative embodiments, are drawn to a composition comprising a detergent at a concentration that is at a concentration that is less than its CMC, including without limitation 99% or about 99%, 95% or about 95%, 90% or about 90%, 80% or about 80%, 75% or about 75%, 60% or about 60%, 50% or about 50%, 40% or about 40%, 30% or about 30%, 20% or about 20%, 10% or about 10%, and 1% or about 1% of its CMC.

Although CMC values for many specific detergents are known, it is difficult to predict the CMC for a mixture (aka “blend”) of different detergent molecules, each with their own CMC. The CMC of a blend usually has to be determined empirically, i.e., by direct measurement. Such determinations can be time-consuming and/or expensive, and are not guaranteed to produce satisfactory results. In the invention, a different approach to testing mixtures of detergents and/or surfactants is taken: a formulation for an MS-compatible solubilizer includes at least one component at a concentration which is above its CMC. In related preferred embodiments, methods and compositions of the present invention are drawn to a composition comprising a detergent at a concentration that is at a concentration that is approximately equal (100%) to its CMC, or at a concentration greater than its CMC, including without limitation 101% or about 101%, 110% or about 110%, 125% or about 125%, 150% or about 150%, 175% or about 175%, 2× or about 2×, 3× or about 3×, 4× or about 4×, 5× or about 5×, 6× or about 6×, 7× or about 7×, 8× or about 8×, 9× or about 9×, 10× or about 10×, 25× or about 25×, 100× or about 100×, of its CMC.

The Molecular Weight (MW) of a specific micelle can be calculated by multiplying the MW of a monomer of the detergent times the micelle's aggregation number. The MW of a particular micelle is of interest in some aspect of the invention, including dialysis. More specifically, a dialysis membrane can have a molecular weight cut-off; that is, only molecules below a certain MW can freely pass through the membrane. The MW of a micelle can be much larger than that of the detergent monomer of which it is composed. One of the discoveries of the invention is that the concentration of a detergent can be manipulated during dialysis in order to achieve a desired effect.

Further, when proteins or peptides suspended or dispersed in common detergents that are incompatible with MALDI-MS are carefully exchanged with MS-compatible surfactant blends, the aforementioned suspensions or dispersions can become compatible with MALDI-MS analyses. That is, co-mixing these detergent blends with very harsh detergents such as Triton X100 seems to shift the size distribution of micelles toward lower aggregates. Whereas the original Triton X100 micelles are intractable, these smaller aggregates or the free monomers arising from addition of the surfactant blends of this invention can be removed from the original solution by ultrafiltration. The process can be carried out by dialysis on conventional ultrafiltration membranes.

Table 2 shows characteristics, including MW, Aggregation Number and Micellar MW of several representative but non-limiting detergents.

TABLE 2
CRITICAL MICELLE CONCENTRATION (CMC) AND MOLECULAR
WEIGHT OF MICELLES FOR SEVERAL DETERGENTS

| | | | | |
|---|---|---|---|---|
| | MW | CMC | AGGREGATION | MW |
| DETERGENT NAME | (MONOMER) | (mM)* | NUMBER | (MICELLE)** |

NON-IONIC DETERGENTS

| | | | | |
|---|---|---|---|---|
| APO-12 | 246.4 | 0.568 | 2,232 | 549,965 |
| TRITON X-100 (tert-C8-Ø- | 650 (avg) | 0.3 | 140 | 90,000 |
| E9.6) | | | | |
| TWEEN 80 (C18:1- | 1310 (avg) | 0.012 | 58 | 75,980 |
| sorbitan-E20) | | | | |
| Digitonin | 1229.3 | | 60 | 70,000 |
| Nonidet P-40 (NP-40) | 603.0 | 0.05-0.3 | 100-155 | 60,300-93,465 |
| n-Dodecyl-β- | 348.5 | 0.13 | | 70,000 |
| D-glucopyranoside | | | | |
| n-Dodecyl-beta-D- | 348.5 | 0.15 | 98 | 70,000 |
| maltoside | | | | |
| APO-10 | 218.3 | 4.6 | 131 | 28,597 |
| n-Octyl-beta-D- | 292.4 | 25 | 27 | 7,895 |
| glucopyranoside | | | | |

IONIC DETERGENTS

| | | | | |
|---|---|---|---|---|
| Lysophosphatidyl-choline | 495.7 | 0.007 | 186 | 92,000 |
| (16:0) | | | | |
| Digitonin | 1229 | 0.087 | 60 | 70,000 |
| CTAB (Cetyltrimethyl- | 364.5 | 1.0 | 170 | 62,000 |
| ammonium bromide) | | | | |
| Tetradecyltrimethyl- | 336.4 | 3.5 | 81 | 27,000 |
| ammonium bromide | | (30° C.) | | |
| (TDTAB) | | | | |
| Sodium n-dodecyl sulfate | 288.5 | 2.30 | 84 | 24,200 |
| (SDS, Lauryl sulfate, Na+ | | | | |
| salt) | | | | |
| Taurodeoxycholic acid, | 521.7 | 2.7 | 8 | 4,200 |
| Na+ salt | | | | |
| Taurocholic acid, Na+ salt | 537.7 | 3.3 | 4 | 2,150 |
| (20 mM Na+) | | | | |
| Deoxycholic acid, Na+ salt | 414.6 | 1.5 | 5 | 2,000 |
| (DOC) | | | | |
| Cholic acid, Na+ salt | 430.6 | 4 | 3 | 1,200 |
| Glycocholic acid, Na+ salt | 487.6 | 7.1 | 2.1 | 1,000 |
| Glycodeoxycholic acid, | 471.6 | 2.1 | 2.1 | 1,000 |
| Na+ salt | | | | |
| Lauroylsarcosine, Na+ salt | 293.4 | | 2 | 900 |
| (Sarkosyl) | | | | |

ZWITTERIONIC DETERGENTS

| | | | | |
|---|---|---|---|---|
| ZWITTERGENT 3-16 | 391.6 | 0.01-0.06 | 155 | 60,700 |
| ZWITTERGENT 3-14 | 363.6 | 0.1-0.4 | 83 | 30,200 |
| ZWITTERGENT 3-12 (3- | 335.6 | 2-4 | 55 | 18,500 |
| Dodecyl-dimethylammonio- | | | | |
| propane-1-sulfonate) | | | | |
| Lauryldimethylamine oxide | 229.4 | 1-3 | 76 | 17,000 |
| (LADAO, LDAO, Empigen | | | | |
| OB) | | | | |
| ZWITTERGENT 3-10 | 307.6 | 25-40 | 41 | 12,600 |
| CHAPSO | 630.9 | 8 | 11 | 9,960 |
| BigCHAP | 878.1 | 3.4 | 10 | 8,800 |
| CHAPS | 614.9 | 6-10 | 10 | 6,150 |

*CMC at 50 mM Na⁺ unless otherwise stated.
**Micelle MW = (Monomer MW) × (Aggregation Number).

Membrane proteins of interest are often solubilized by the presence of a detergent, including without limitation the detergents presented in Table 3.

TABLE 3
EXEMPLARY DETERGENTS FOR MEMBRANE PROTEIN STUDIES

| COMMON | | MW | CMC | AGGREGATION | MW |
|---|---|---|---|---|---|
| NAME | CHEMICAL NAME | MONOMER | (mM) | NUMBER | (MICELLE) |
| Triton X- | tert. C8 phenyl | 628 | 0.24-0.34 | 100-155 | 62,800-97,340 |
| 100 | poly ethylene | | | | |
| | glycole (9-10) | | | | |
| octyl-POE | polydisperse octyl | 400 | 6.6 | 57 | 22,800 |
| | oligo oxyethylene | | | | |
| SDS | C12-sulfate-Na+ | 288 | 8.2 | 62 | 17,856 |
| b-OG | C8-b-D- | 292 | 25 | 27 | 7,884 |
| | glucopyranoside | | | | |
| CHAPS | 3-cholamido propyl | 615 | 8 | 10 | 6,150 |
| | dimethyl ammonio- | | | | |
| | 1-propane sulfate | | | | |

Detergents and non-detergent surfactants include without limitation those provided in the examples herein.

### Lipids

Although they are not detergents per se, lipids are, like detergents, amphipathic surface-active molecules. Generally, lipids are any of a variety of oily or greasy organic compounds found as major structural components of living cells; they are insoluble in water but soluble in organic solvents such as alcohol and ether, and include the common fats, cholesterol and other steroids, phospholipids, sphingolipids, waxes, and fatty acids.

As regards their chemical structure, lipids are fatty acid esters, a class of relatively water-insoluble organic molecules. There are three forms of lipids: phospholipids, steroids. and triglycerides. Lipids consist of a polar or hydrophilic (attracted to water) head and one to three nonpolar or hydrophobic tails. Since lipids have both functions, they are called amphiphilic. The hydrophobic tail consists of one or two (in triglycerides, three) fatty acids. These are unbranched chains of carbon atoms (with the correct number of H atoms), which are connected by single bonds alone (saturated fatty acids) or by both single and double bonds (unsaturated fatty acids). The chains are usually 14-24 carbon groups long. For lipids present in biological membranes, the hydrophilic head is from one of three groups: (1) glycolipids, whose heads contain an oligosaccharide with 1-15 saccharide (sugar) residues; (2) phospholipids, whose heads contain a positively charged group that is linked to the tail by a negatively charged phosphate group; and (3) sterols, whose heads contain a planar steroid ring, for example, cholesterol.

### Use of Solubilizers

The majority of detergents cannot be tolerated in ESI-MS, since they suppress the ESI process. The use of detergents in MALDI-TOF MS varies. Using model (water soluble) proteins, it has been shown that non-ioinic detergents (Triton X-100 and b-octylglucoside) can be tolerated at lower concentrations (Bomsen et al., Rapid Commun Mass Spectrom. 11:603, 1997). SDS, commonly used to solubilze proteins e.g. during PAGE, is a strong (denaturing) anionic detergent. At low concentrations (0.01-0.05%) SDS was shown to be detrimental to MALDI spectra (Jeannot et al., J Am Soc Mass Spectrom. 10:512, 1999). Nevertheless, for hydrophobic proteins and peptides, inclusion of SDS can sometimes improve the signal to noise (S/N) ratio (Zhang et al., Anal Chem. 73:2968, 2001; Breaux et al., Anal Chem. 72:1169, 2000) and SDS, as well as other anionic detergents, provide acceptable spectrum quality for a number of water-soluble proteins and peptides (Amado et al., Anal Chem 69:1102, 1997).

Lukas et al. (Anal Biochem. 301:175, 2002) state that the efficiency of extraction of a hydrophobic protein (nAChR) from membranes was not markedly different for the detergents tested, but quality and signal size of mass spectra were influenced by the composition and concentration of the detergents, as well as the concentration of protein and MALDI matrix composition. Lukas et al. state that their best spectra were obtained for samples solubilized in Triton X-100 and assayed by use of a sinapinic acid matrix.

At the time a MS-compatible detergent of the invention contacts the analyte, the MS-compatible detergent can be at a concentration of at least about 25%, about 50%, about 75%, about 80%, about 85%, about 90%, about 95%, about 100%, or greater than about 100%, for example about 110%, about 125%, about 150%, about 200%, about 300%, about 400%, or about 500% of its critical micelle concentration (CMC). In some preferred embodiments, the MS-compatible detergent can be at a concentration of at least 75%, 80%, 85%, 90%, 95%, 100%, or greater than 100%, for example 110%, 125%, 150%, 200%, about 300%, about 400%, or about 500% of its CMC when it contacts a sample that contains one or more analytes and subsequently the sample can be diluted, for example, to bring the concentration of the solubilizer to a concentration below its CMC, prior to performing mass spectrometry.

A matrix compound can be added to a sample or analyte prior to adding an MS compatible solubilizer to a sample or analyte or after adding an MS solubilizer to a sample or analyte. A matrix compound added to a sample after adding an MS solubilizer to a sample can be added before or after any dilution of the sample/solubilizer solution that may be performed.

During the time of analyte:matrix crystal formation for mass spectrometry analysis, the MS-compatible detergent is preferably at a concentration at or below its CMC, but this is not a requirement of the present invention. For example, the concentration of an MS compatible detergent can be at least about 1%, at least about 5%, about 10%, about 25%, about 30% about 35%, about 40%, about 45%, about 50%, about 55%, about 60%, about 65%, about 70%, about 75%, about 80%, about 85%, about 90%, about 95% about 100%, or greater than 100%, for example 110%, 125%, 150%, or 200% of its critical micelle concentration (CMC). In certain preferred embodiments, the MS-compatible solubilizer is at a concentration of less than 100%, of its CMC immediately prior to crystal formation and mass spectrometry analysis.

### MS-Compatible Solubilizer Formulations

Typically, an MS-compatible solubilizer of the invention comprises one or more MS-compatible detergents or non-detergent surfactants, or blends (mixtures) thereof. Provided herein are illustrative methods for identifying detergents, non-detergents and blends thereof that can be used as MS-compatible solubilizers.

In some embodiments, a MS-compatible solubilizer of the invention comprises one or more chemical compounds identified by structure and chemical formula herein. These include without limitation alkyl glycosides, sulfobetaine detergents, non-detergent sulfobetaines (NDSBs), bile acids and Rabilloud detergent variants. In particular, see Example 15, Illustrative Detergents, Non-Detergents and Other Compositions for MS-Compatible Solubilizers.

In some embodiments, a MS-compatible solubilizer of the invention comprises one or more MS-compatible non-detergent surfactants. The MS-compatible non-detergent surfactant is a compound that is capable of forming bonds with a hydrophobic portion of a molecule and forming bonds with hydrophilic solvent molecules as well, thus preventing aggregation and precipitation of the hydrophobic molecule. Thus, non-detergent surfactants enhance the solubility of hydrophobic molecules by simultaneously forming bonds with the hydrophobic molecule and the surrounding solvent molecules but, unlike detergents, lack the ability to aggregate into micellar structures.

Optimal concentrations of solubilizers in a formulation can be determined empirically using tests set forth in the example for the effects of matrix additives on signal-to-noise ratio of mass spectra, on the size of adduct cluster peaks in mass spectra, on the analyzable surface area of a MALDI sample, or on the stability of an analyte:matrix crystal used in MALDI-MS.

Non-limiting examples of non-detergent surfactants are non-detergent sulfobetaines (NDSBs), such as NDSB-195, NDSB-201, NDSB-211, NDSB-221, NDSB-223, and NDSB-256. In some embodiments, a MS-compatible solubilizer of the invention comprises an NSBD at a concentration of from about 5 mM to about 1 M, or from about 10 mM to about 0.8 M, or from about 50 mM to about 700 mM, or from about 100mM to about 600 mM. For example, NSBD-201 can be present in a solubilizer at a concentration of from about 125 mM to about 500 mM, for example 250 mM to 500 mM, and can be present in a solution with analyte to be analyzed by MS at a concentration of from about 10 mM to about 500 mM, preferably between about 20 mM and about 400 mM, when in solution with the analyte immediately prior to crystal formation. In one embodiment, an MS-compatible solubilizer comprises NSBD-201 at a concentration of about 250 mM when in solution with the analyte immediately prior to crystal formation and mass spectrometry analysis. In another embodiment, an MS-compatible solubilizer comprises NSBD-201 at a concentration of about 25 mM when in solution with the analyte immediately prior to crystal formation and mass spectrometry analysis.

In some embodiments, a MS-compatible solubilizer of the invention comprises one or more organic co-additives. Such organic co-additives include without limitation phospholipids, fatty acids, steroid compounds and organic solvents. An MS-compatible solublizer solution can comprise one or more buffers, acids, bases, or salts, such as, for example, ammonium bicarbonate at a concentration of from 10 to 100 mM.

In some embodiments, a MS-compatible solubilizer of the invention comprises one or more of the specific mixtures of MS-compatible detergents and/or MS-compatible non-detergent surfactants disclosed herein. Various combinations can be empirically tested. For example, a combination of ASB-C8Ø, Octyl-beta-D-1-thioglucopyranoside, n-Dodecanoylsucrose, and SB14 can be used. The concentrations of the detergent components can be such that one or more detergents is contacted with an analyte at a concentration above its CMC. Examples of commercially available MS-compatible solubilizers that can be used in this and other methods and compositions of the present invention are Invitrosol A, Invitrosol B and Invitrosol LC (Invitrogen, Carlsbad, Calif.).

The invention also provides stock solutions of MS-compatible solubilizers. A stock solution is one that must be diluted to achieve a desired final working concentration. For example, a 5× solution of a MS-compatible solubilizer of the invention can comprise ASB-C8Ø at from about 0.05 to about 10 mM, and preferably from about 0.1 to about 0.5 mM; Octyl-beta-D-1-thioglucopyranoside at from about 10 to about 500 mM and preferably from about 20 mM to about 250 mM; n-Dodecanoylsucrose from about 0.5 to about 20 mM; and SB14 at about 0.1 to about 10 mM and preferably from about 0.2 mM to about 5 mM. An exemplary 5× solution of a MS-compatible solubilizer of the invention (Invitrosol A) comprises ASB-C8Ø at 0.125 mM; Octyl-beta-D-1-thioglucopyranoside at 50 mM; n-Dodecanoylsucrose at 3.8 mM; and SB14 at 1 mM. When in contact with an analyte immediately prior to MS analysis, the exemplary solubilizer can have a concentration of ASB-C8Ø or from about 0.01 to about 0.5 mM, more preferably from about 0.02 mM to about 0.1 mM, or in one embodiment, about 0.025 mM; a concentration of Octyl-beta-D-1-thioglucopyranoside of from about 1 mM to about 50 mM, more preferably from about 5 mM to about 25 mM, or in one embodiment, about 10 mM; a concentration of n-Dodecanoylsucrose from about 0.1 to about 10 mM, more preferably from about 0.5 to about 5 mM, or or in one embodiment, about 0.76 mM; and a concentration of SB-14 of from about 0.05 mM to about 1 mM, more preferably from about 0.1 mM to about 0.5 mM, or about 0.2 mM. The solubilizer formulation can also include a buffer, such as, for example, ammonium bicarbonate, at a pH between about 7.5 and about 8, at a concentration of between about 10 mM and about 100 mM after contact with an analyte.

Another stock solution of MS-compatible solubilizers can be a 5× solution comprising NDSB-201, NDSB-256, and SB-14. For example, a 5× solubilizer solution can comprise NDSB-201 from about 25 mM to 650 mM, and preferably from about 50 mM to 300 mM; NDSB-256 from about 25 mM to 650 mM, and preferably from about 50 mM to 300 mM; and SB-14 from about 0.05 mM to about 1mM, and preferably from about 0.1 mM to about 0.5 mM. An exemplary 5x solution of a MS-compatible solubilizer of the invention (Invitrosol LC) comprises NDSB-201 at 125 mM, NDSB-256 at 125 mM, and SB-14 at 1.1 mM. An alternative 5× formulation can be NDSB-201 at 250 mM, NDSB-256 at 250 mM, and SB-14 at 2.2 mM.

An exemplary solubilizer can that is compatible with liquid chromatography can have a final concentration when in contact with the analyte just prior to MS analysis of, for example, from about 10 mM to about 100 mM NDSB-201, preferably from about 20 mM to about 60 mM, for example, a concentration of about 25 mM or about 50 mM; from about 10 mM to about 100 mM NDSB-256, preferably from about 20 mM to about 60 mM, for example, a concentration of about 25 mM or about 50 mM; and from about 0.1 mM to about 1 mM SB-14, preferably from about 0.1 mM to about 0.5 mM, for example, about 0.22 mM.

As another non-limiting example, a 2× solution of a MS-compatible solubilizer of the invention comprises from about 10 mM to about 1 M NDSB-201, preferably from about 20 mM to about 800 mM NDSB-201, and more preferably from about 100 mM to about 600 mM NDSB-201. An exemplary 2x solution of a solubilizer of the present invention (Invitrosol B) comprises 500 mM NDSB-201.

Chaotropes may be used with the matrix additives of the invention. Surfactants used in solubilization solutions can act synergistically with chaotropes to solubilize hydrophobic proteins, such as membrane proteins. Chaotropic agents unfold proteins, thereby exposing hydrophobic regions of the protein which can cause undesirable aggregation, precipitation, or adsorption to a solid surface. The surfactant binds to these hydrophobic domains, thus helping to keep the protein solubilized. These include without limitation urea, thiourea and guanidine hydrochloride, typically at concentrations of from about 1 M to about 10 M, e.g., about 1, about 2, about 3, about 4, about 5, about 6, about 7, about 8, about 9 or about 10 M; or at 1, 2, 3, 4, 5, 6, 7, 8, 9 or 10 M.

### MS-Compatible Sorbents

In another embodiment, the invention provides MALDI matrix additives that support and/or promote the formation of small analyte:matrix crystals in thin layers. In this embodiment, the MALDI matrix additive is preferably a MS-compatible sorbent. Non-limiting examples of MS-compatible sorbents are silicas, aluminia, germanium oxide, indium tin oxide, metal oxides, chlorides, sulfates, phosphates, carbonates and fluorides; polymer based oxides, chlorides, sulfates, carbonates, phosphates or fluorides; diatomaceous earth; graphite or activated charcoal; and titania, gold and activated gold. In some embodiments, silica is preferred.

A MS-compatible sorbent of the invention is typically, but need not be, non-volatile. A MS-compatible sorbent of the invention can be provided as a colloid, although other forms can be used as well.

A MS-compatible sorbent of the invention, when present at an effective amount, has one or more characteristics: (a) it increases the signal-to-noise ratio by at least about 5%; (b) it increases by at least about 5% one or more adduct cluster peaks of a molecule that forms adduct with ions; (c) it increases the stability of an analyte:matrix crystal by at least about 5%; (d) it diffracts and/or reflects the incident laser beam in a MALDI matrix comprising one or more additives to a degree sufficient to alter the fluence by at least about 1%; (e) it diffracts and/or reflects the incident laser beam in a MALDI matrix comprising one or more additives to a degree sufficient to alter the fluence at least about 10 Joules/square centimeter; and/or (f) it increases, by at least about 1%, the amount of energy that is absorbed by a MALDI matrix.

The use of a MALDI matrix additive of the invention in MALDI-TOF MS results in a higher number of analyte molecules available for desorption and/or ionization. The MALDI matrix additives of the invention modify the size and/or morphology of matrix crystals and matrix:analyte crystals, such that the surface area available for laser irradiation (the “analyzable surface area”) is increased.

In certain illustrative examples silica particles can be added to the matrix to reduce matrix background noise. For example, a 1:1 ratio of silica particles:matrix can be used. The silica particles can be provided in a variety of chemical forms including, for example, SiO₂.

The matrix additive can be provided in the form of a colloid, such as a colloidal solution or a resin, as a component of a diluent into which a MALDI matrix or an analyte is dissolved, or in solid form. Two or more matrix additives, and/or types of matrix additives, can be used in any given composition or method of the invention.

In some embodiments, a composition comprising one or more of the MS-compatible sorbents of the invention further comprises one or more ion-sequestering molecules. Non-limiting examples include sulfonates and zwitterionic surfactants. Ion-sequestering molecules can be introduced into the MALDI sample and/or MS matrix in colloidal form.

In some embodiments, a MS-compatible sorbent of the invention comprises one or more chemical compounds or compositions disclosed herein. In particular, see Example 17, MaxIon AC: Other Compositions.

In some embodiments, a MS-compatible sorbent of the invention comprises is or comprises a resin. The resin can, by way of non-limiting example, be LiChrosorb®, LiChrospher®, LiChroprep®, LiChroprep® or Purospher®. More specifically, the resin can be LiChrosorb® 5 μm, 5 μm RP8, 5 μm RP18, LiChrosorb® 5 μm RP-Select B, LiChrosorb® 5 μm DIOL, LiChrosorb® 10 μm RP18, LiChrosorb® 10 μm RP8, LiChrosorb® 10 μm RP18, LiChrosorb® 5 μm Si60 or Silica Gel 60 RP-18.

In some embodiments, a MS-compatible sorbent of the invention comprises is a composition comprising particles. The particles can comprise silica. The particles preferably have at least one dimension >1 micron. The particles can have irregular or regular (e.g., spherical) shapes.

### MS-Compatible Buffers

In another embodiment, a MALDI matrix additive of the invention is or comprises a MS-compatible buffer. In some embodiments, the MS-compatible buffer is a morpholino-sulfonic acid.

Non-limiting examples of MS-compatible buffers include, in no particular order: 2-(n-morpholino)ethane sulfonic acid (MES); 4-(n-morpholino)butane-sulfonic acid (MOBS); 3-(n-morpholino)propane-sulfonic acid (MOPS); and 3-(n-morpholino)-2-hydroxypropanesulfonic acid (MOPSO).

Certain illustrative MS-compatible buffers have the structure:

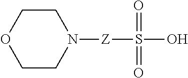

wherein Z═[CH₂]ₐ—CH—OH]_{b}—[CH₂]_{c}, and wherein a=0 to 25, b=0 to 25 and c=0 to 25; with the exception that, if b=0, a and c cannot both be 0. In some embodiments, b=0 and c=0.

In some embodiments, a MS-compatible buffer of the invention comprises one or more chemical compounds or compositions disclosed herein. In particular, see Example 25: MaxIon SA: Structures of MES, MOPS and Related Compounds. The buffers can be used at any concentration that is compatible with MS, for example, from about 1 mM to about 1M, or from about 10 mM to about 500 mM, or from about 20 mM to about 250 mM. The MS-compatible buffers of the invention improve the stability of a matrix crystal, and preferably reduce the signal-to-noise ratio in MS, particularly where multiple laser shots are used on a sample.

II. Methods of Using MS-Compatible Reagents in Mass Spectrometry

In one aspect, the invention comprises methods of using MS-compatible reagents and/or compositions to (i) improve the solubility of an analyte by at least about 5% during one or more sample processing procedures, (ii) improves the solubility of an analyte by at least about 5% in a composition comprising a matrix, (iii) improve the stability of an analyte:matrix crystal by at least about 5%, (iv) increase the analyzable surface area of an analyte-matrix crystal by at least about 1%, (v) increase the signal-to-noise ratio by at least about 5%, and/or (vi) diminish by at least about 5% one or more adduct cluster peaks of a molecule that forms adduct with ions.

In some preferred methods, the MS-compatible reagent is a MALDI matrix additive that is present in a matrix crystal during MS analysis. The MALDI matrix additive can be contacted with the sample or analyte prior to mixing the sample with the matrix, or can be provided with the matrix prior to contacting the sample or analyte with the matrix.

The methods of the invention include without limitation processing and/or preparing samples for MS, such as but not limited to MALDI-MS and LC/MS, using one or more of the matrix additives of the invention. Included are methods of solubilizing biomolecules for MS analysis; methods of analysis using MALDI-TOF MS; methods of stabilizing analyte:matrix crystals for MALDI-MS; and methods of preparing MS target surfaces.

The invention provides methods of using the MS-compatible reagents of the invention, such as MALDI matrix additives of the invention, in MALDI-TOF MS and other procedures for detecting, quantifying and/or studying the properties of a molecule, such as an analyte or a biomolecule.

In one aspect, the invention is drawn to methods of MALDI-TOF MS analysis of a MS target molecule comprising contacting the molecule(s) with one or more of the MALDI matrix additives of the invention, and performing MALDI-MS on one or more target molecules.

In another aspect, the invention provides methods and compositions for preparing a target sample for MALDI-TOF MS. The target sample is or comprises a crystal comprising matrix and additive molecules, or a matrix crystal comprising matrix, analyte and additive molecules. In some embodiments, the matrix and analyte are combined in a single step, followed by addition of the MS matrix additive. In other embodiments, the matrix and additive are combined in a single step, followed by addition of analyte. In some embodiments, the matrix, analyte and additive are combined in a single step. The latter embodiment, which is or comprises a single-step matrix crystallization reaction, is preferred in some instances.

The invention provides a method of obtaining a MALDI MS spectrum of an analyte, comprising: contacting an analyte with, in either order or in combination, a MALDI matrix; and one or more of 1) a MS-compatible solubilizer, 2) a MS-compatible sorbent, and 3) a MS-compatible buffer. The method further includes co-precipitating said analyte with the MALDI matrix, thus generating analyte:matrix crystals; subjecting said analyte:matrix crystals to laser irradiation, thus generating analyte ions; and detecting and quantifying the analyte ions to generate a MALDI-MS spectrum of said analyte. In some aspects of these methods, the analyte is a protein or peptide.

In other aspects, the invention provides methods and compositions for producing stable matrix/analyte mixtures that can be spotted and stored on a MALDI target surface, or otherwise stored, for future analysis. Matrix:analyte crystals, compositions comprising such crystals, and methods of making and using such crystals are provided for in this and other aspects of the invention. In one aspect, the invention is drawn to an analyte:matrix crystal comprising one or more MS-compatible compositions. The MS-compatible composition can be a MS-compatible solubilizer, or a composition comprising one or more MS-compatible non-volatile MALDI additives. Preferably the crystals are stable, preferably under a variety of conditions.

The method includes: contacting the analyte with a MALDI matrix and one or more of 1) a MS-compatible solubilizer, 2) a MS-compatible sorbent, and 3) a MS-compatible buffer; and co-precipitating said analyte with said MALDI matrix to generate analyte:matrix crystals for MALDI MS analysis.

In some aspects, the invention is drawn to methods that comprise or involve a MS matrix additive for improving the signal-to-noise ratio during MALDI-TOF MS analysis of molecules including without limitation peptides, proteins, oligonucleotides, oligosaccharides, phospholipids, polymers, and small organic molecules. Any of these molecules can be from a biological source (“biomolecules”) or from in vitro chemical synthesis (“synthetic molecules”).

In one embodiment of this aspect of the invention, the invention provides methods and compositions for creating and/or enhancing analyzable surface areas for laser irradiation during MALDI-TOF MS. Additionally or alternatively, the extent of analyte desolvation is increased. The formation of small crystals in thin layers, which is enhanced by the additive, results in more efficient desolvation of analyte molecules, thus maximizing the number of “de-sorbable” analyte molecules.

A matrix additive can act as an ion-exchanger that reduces or preferably eliminate the undesirable effects of ions, such as cations, including monovalent cations, and adducts of these and other ions, from MALDI spectra. More specifically, in the case of a MS-compatible sorbent, the invention provides formulations to maximize the number of deprotonated SiO₂ sites in a matrix, or in a composition used to prepare, treat or wash a crystallized matrix. These sites act as ion-exchangers for the removal of undesirable contaminants that are or result from ions, such as cations, more specifically monovalent cation contaminants.

A desirable result of a MALDI matrix additive's positive effects on crystal size and morphology and/or capacity to act as a cation-exchanger, include without limitation: (a) improvement of signal-to-noise; (b) suppression of matrix background noise; and/or (c) reduction or elimination of ion-adducts, such as monovalent cation-adducts, and/or detrimental effects resulting therefrom.

The methods of the invention provide for the selective reduction, preferably elimination, of ion adducts, such as cation adducts, including monovalent cation adducts. Thus, one measure of an effective amount of an matrix additive of the invention involves its ability to selectively reduce and preferably eliminate monovalent cation adducts. Protein adducts are particularly undesirable to those studying a proteome, as it causes both the loss of molecules of interest (proteins) and production of contaminants (adducts). Both events complicate the target sample, and both can introduce inaccuracy and/or imprecision in the MS spectra. The adduct cluster peaks repeat at intervals of (M−1) Da, where M is the molecular mass of the cation. The invention provides for the reduction or elimination of the adduct cluster peaks in a MALDI-TOF MS spectrum. Preferably, the M−1 adduct cluster peak is diminished by at least about 10% upon addition of a MALDI matrix additive of the invention, preferably by at least 50% or about 50%, most preferably by 95% or about 95%, to about 100%. The M−1 adduct cluster peak is completely diminished at 100%, but solubilizers that result in near complete elimination of the peak are also within the scope of the invention. For example, the M−1 adduct cluster peak is nearly completely diminished at about 90% or 90%, about 95% or 95%, about 96% or 96%, about 97% or 97%, about 98% or 98%, about 99% or 99%. A reference standard molecule having a known response to the solubilizing agent can be used to confirm and measure the desirable properties resulting from the presence of the solubilizer. One such reference standard molecule for effects of solubilizers on adducts is bradykinin, which can be tested using the compositions, methods and conditions of the invention as described in the Examples.

Accordingly, provided herein is a method to analyze a molecules, such as a protein, using MALDI-TOF mass spectroscopy according to the above method, wherein a monovalent cation is present in a solution that includes the molecule at the time the molecule is contacted with a MALDI matrix additive of the invention and the mass spectroscopy matrix that includes contacting the molecule with a MALDI matrix additive and a mass spectroscopy matrix, and analyzing the molecule using MALDI-TOF. The MALDI matrix additive can be a MS-compatible solubilizer, such as a MS-compatible detergent and/or a MS-compatible non-detergent surfactant.

The invention provides compositions and methods for preparing a protein for MALDI-TOF analysis, the method comprising contacting a composition comprising the protein, in any order or combination, with (a) at least one MALDI matrix additive of the invention and (b) at least one enzyme, such as a protease or a protein-modifying enzyme. By way of non-limiting example, in the case of peptide mass fingerprinting (PMF), the enzyme is a protease. In a more specific embodiment, the invention provides compositions and methods for preparing a protein having one or more hydrophobic regions for MALDI-TOF analysis, the method comprising contacting a composition comprising said protein, in any order or combination, with (a) at least one MS-compatible solubilizer and (b) at least one enzyme, such as a protease or a protein-modifying enzyme.

The invention provides methods of obtaining a MALDI MS spectrum of an analyte that include: 1) contacting an analyte with, in any order or combination, a MALDI matrix; and one or more of a MS-compatible solubilizer, a MS-compatible sorbent, and a MS-compatible buffer; and, at least one protease to generate one or more peptides. The method further includes co-precipitating the one or ore peptides with the MALDI matrix to generate analyte:matrix crystals; subjecting the analyte:matrix crystals to laser irradiation to generate peptide analyte ions, and detecting and quantifying the peptide analyte ions to generate a MALDI-MS spectrum of the one or more peptides. The analyte can be any type of analyte, such as, for example, a nucleic acid, a carbohydrate, or a protein.

The invention provides methods of determining one or more amino acid sequences of a protein analyte that include: 1) contacting a protein analyte with, in any order or combination, a MALDI matrix; and one or more of a MS-compatible solubilizer, a MS-compatible sorbent, and a MS-compatible buffer; and, at least one protease to generate one or more peptides. The method further includes co-precipitating the one or ore peptides with the MALDI matrix to generate analyte:matrix crystals; subjecting the analyte:matrix crystals to laser irradiation to generate peptide analyte ions, and detecting and quantifying the peptide analyte ions to generate a MALDI-MS spectrum of the one or more peptides, and using the MALDI-MS spectrum to determine the sequences of the one or more peptides, where the sequences of the peptides are sequences of the protein analyte.

The invention provides methods of determining an amino acid sequence of a protein analyte that binds to a ligand that include: 1) contacting a protein analyte with, in any order or combination, a MALDI matrix; and one or more of a MS-compatible solubilizer, a MS-compatible sorbent, and a MS-compatible buffer; and, 2) contacting a second sample comprising said protein analyte with, in any order or combination, a MALDI matrix; one or more of a MS-compatible solubilizer, a MS-compatible sorbent, and a MS-compatible buffer; and a ligand of the protein analyte.

The method further includes 3) independently contacting the first and second samples with a protease, to generate a first set of one or more peptides and a second set of one or more peptides and 4) independently co-precipitating the rist and second set of peptides with the MALDI matrix to generate first and second analyte:matrix crystals. The method further includes 5) subjecting the analyte:matrix crystals independently to laser irradiation, thus generating first and second sets of peptide analyte ions and 6) detecting and quantifying the peptide analyte ions, to generate a MALDI-MS spectrum of the first and second sets of peptides; and using the MALDI-MS spectrum to determine the amino acid sequences of the first and second sets of peptides, in which an amino acid sequence depleted in the sequences of the second set set relative to the first set is an amino acid sequence of a protein analyte that binds the ligand.

In one embodiment of this aspect, the invention provides methods of preparing a hydrophobic molecule for MS analysis, the methods comprising contacting a composition comprising said hydrophobic molecule with at least one MS-compatible solubilizer. The hydrophobic molecule, for example, can be a membrane protein. Using the compositions and methods provided herein, membrane proteins can be analyzed using mass spectrometry for chemically modified sites, ligand binding sites, and component peptide sequences. In these aspects, the present invention expands the useful applications of MS to hydrophobic molecules including membrane proteins.

Optionally, composition can comprise one or more enzymes, one or more chaotropes, and/or one or more co-additives. Co-additives include without limitation phospholipids, fatty acids, cholesterol, steroid compounds and organic solvents. Such co-additives help to separate hydrophobic molecules from other molecules, including molecular complexes or other hydrophobic molecules in a sample. By way of non-limiting example, when the analyte is a protein, co-additives can be used to help separate the protein of interest from a molecular complex, or to help displace a membrane protein of interest from membranes.

In another embodiment, the invention provides compositions and methods of preparing a sample comprising a protein, such methods comprising (a) subjecting a sample comprising the protein to a process that at least partially separates the protein from other molecules in the sample, to generate a partially purified protein, and (b) contacting a composition comprising the partially purified protein with MALDI matrix additive of the invention, thereby generating a sample comprising the protein suitable for MALDI-TOF analysis. An enzyme, such as a protease, and/or a matrix suitable for MALDI-TOF may also be added at (b) or some other point in sample preparation. In instances wherein the sample comprises a protein having one or more hydrophobic regions for MALDI-TOF analysis, a preferred MALDI matrix additive is a MS-compatible solubilizer.

The invention provides methods of obtaining a MS-MALDI spectrum of a protein analyte, methods of determining one or more amino acid sequences of a protein analyte, methods for identifying an amino acid sequence of a protein analyte that binds to a ligand, methods for identifying an amino acid sequence of a protein analyte that is chemically modified by a protein-modifiying enzyme. Protein-modifiying enzymes include without limitation kinases, phosphatases, glycosylases, deglycosylases, and combinations and complexes thereof.

The invention provides compositions and methods for preparing a protein for MALDI-TOF analysis, the method comprising contacting a composition comprising the protein, in any order or combination, with (a) at least one MALDI matrix additive of the invention and (b) at least one enzyme, such as a protease or a protein-modifying enzyme. By way of non-limiting example, in the case of peptide mass fingerprinting (PMF), the enzyme is a protease. In a more specific embodiment, the invention provides compositions and methods for preparing a protein having one or more hydrophobic regions for MALDI-TOF analysis, the method comprising contacting a composition comprising said protein, in any order or combination, with (a) at least one MS-compatible solubilizer and (b) at least one enzyme, such as a protease or a protein-modifying enzyme.

In another embodiment, the invention provides compositions and methods for identifying a region on a molecule that binds to a region on a ligand, comprising contacting the molecule and/or the ligand with at least one MALDI matrix additive of the invention, thereby generating a sample suitable for MALDI-TOF analysis, and subjecting the sample to MALDI-TOF analysis. The molecule and/or the ligand can be hydrophobic, or comprise at least one region that is hydrophobic, in which case a preferred MALDI matrix additive is a MS-compatible solubilizer of the invention.

In another embodiment, the invention provides compositions and methods for identifying a protein that binds to a ligand, the method comprising (a) contacting, in any order or combination, (i) a sample comprising one or more proteins, (ii) the ligand, (iii) one or more cross-linkers, (iv) a MALDI matrix additive of the invention, and (v) a protease; in order to generate cross-linked peptides, which are cross-linked to the ligand or some portion thereof, and determining the amino acid sequences of the cross-linked peptides by MALDI-MS analysis. The amino acid sequence of the cross-linked peptides comprise all or part of a region on a protein that binds to said ligand. The matrix additive can be any disclosed herein, such as for example, a MS-compatible buffer, sorbent, or solubilizer.

In another embodiment, the invention provides compositions and methods for identifying a protein or region thereof that is chemically modified, said method comprising: (a) contacting, in any order or combination, (i) the protein, (ii) an enzyme that modifies proteins, (iii) a MALDI matrix additive of the invention, and (iv) a protease, in order to generate chemically modified peptides. The amino acid sequences of the chemically modified peptides are determined by MALDI-TOF analysis; these amino acid sequences comprise all or part of a region on a protein that is chemically modified by the enzyme. The matrix additive can be any disclosed herein, such as for example, a MS-compatible buffer, sorbent, or solubilizer.

The invention also provides compositions and methods for extending sequence coverage in peptide-mass fingerprinting, comprising contacting the peptide with a MALDI matrix additive of the invention and performing MALDI-MS on the peptide. In these embodiments, the sequence coverage of the peptide is greater than that of the peptide analyzed by MALDI-MS in the absence of the matrix additive.

The invention provides compositions and methods for inhibiting the formation of protein:ion adducts in a protein, comprising contacting the protein with a MALDI matrix additive of the invention and performing MALDI-MS on the peptide.

The invention also provides compositions and methods for evaluating uncharacterized compounds and compositions for their potential as matrix additives of the invention (e.g., MS-compatible solubilizers, MS-compatible sorbents and/or MS-compatible buffers).

### MALDI-TOF MS

Matrix-assisted laser desorption time-of-flight mass spectrometry (MALDI-TOF MS) typically involves several processes, e.g., matrix formation (co-crystallization), desorption, desolvation and ionization. Matrix formation involves mixing analyte molecules with an excess of matrix molecules and co-crystallizing the two. Typically, the matrix is an acidic aromatic, e.g., sinapinic acid (SA) or alpha-cyano-4-hydroxycinnamic acid (CHCA).

The matrix molecules are selected to be capable of absorbing light at wavelengths compatible with an emitting laser. During laser irradiation, the matrix molecules absorb this energy and are desorbed by photoejection from the target surface. Because it is co-crystallized with matrix molecules, the analyte is released (co-desorbed) with the matrix.

Analyte molecules are complexed with and/or surrounded by matrix molecules as they leave the surface. After a short distance the analyte molecules begin to desolvate and separate from the matrix, allowing them to be ionized.

Ionization can occur as analyte molecules are released and/or after desolvation. Typically, in the gas phase, the analyte molecules are ionized from matrix clusters in a series of collision events. Ions are accelerated into a time-of-flight tube where they are separated by their momentum.

### MALDI Matrices

The selection of matrix varies depending on the nature of the analyte being analyzed. Generally, however, effective MALDI matrices share common physical and chemical characteristics. (1) the matrix must effectively associate with the analyte to break-up intermolecular aggregation of analyte molecules, and to assure even distribution of the analyte within the sample spot; (2) the matrix must be stable under vacuum conditions; (3) the matrix must absorb at wavelengths compatible with the emitting laser. Moreover, the solubility properties of the analyte must match that of the matrix molecule, so that they are soluble within the same volatile solvent. For any given analyte of interest, identification of an optimal matrix, ratio of matrix:analyte, analyte concentration, solvent selection, spotting method, sample preparation conditions and instrument conditions is desirable for best results involving that specific analyte.

Because of the differences in the physicochemical properties between analytes, much of determining optimal conditions for MALDI-MS analysis remains empirical, but there are general guidelines and rules that apply to most if not all samples. Non-volatile solvents such as DMSO or DMF are to be strictly avoided. Similarly, surface-active compounds such as Triton-X100 are to be avoided since they disrupt matrix crystal formation, as do high concentrations of chaotropes such as urea or PEG Generally, low pH (≦about 4) is required for effective crystal formation of organic acid matrices, therefore 0.1% TFA or an equivalent volatile acid is required. If the analyte sample contains contaminants such as salts, or nonvolatile solvents, surfactants or chaotropes, these must be removed by solid-phase extraction or dialysis prior to MALDI-MS.

Beyond the above general advice, conditions vary from analyte to analyte. That is, conditions for MALDI for each specific analyte vary and must be optimized in terms of, e.g., choice of matrix, treatment of the matrix:analyte during MALDI, etc. Optionally, matrix additives might be used, or the matrix or analyte might be pretreated, etc. All these parameters are typically determined empirically. Preparation of samples for MALDI is presently as much art as science.

As used herein, the terms “matrix” and “matrix molecules” refer to the material with which a biomolecule can be combined for MALDI mass spectrometric analysis. Any substance that can absorb light at the laser's wavelength (300-400 nm) and is crystal-forming can be used. Any matrix material, such as solid acids, including 3-hydroxypicolinic acid and alpha-cyano-4-hydroxycinnamic acid (a.k.a. gentisic acid, CHCA, 4-HCCA), and liquid matrices, such as glycerol, known to those of skill in the art for MALDI-TOF MS analyses is contemplated. Materials useful for matrix formulation include without limitation 4-HCCA (a.k.a. CHCA), sinapinic acid (SA), 2,5-dihydroxybenzoic acid (DHBA), 3-hydroxy-picolinic acid (HPA) (all available from, e.g., Sigma-Aldrich, St. Louis, Mo.) and nor-harmane (Sigma). Generally, nor-harmane is prepared as a 10 mg/ml solution in 50% acetonitrile/50% water for aqueous soluble molecules, tetrahydrofuran for polymers and chloroform for lipids.

In general, SA is recommended for preparations of intact hydrophobic proteins and 4-HCCA is recommended for enzymatic digestion of proteins. Sinapinic acid, which is mostly used for analysis of intact proteins, has a fragile crystal structure that becomes ablated during prolonged exposure to the MALDI laser. Thus, this fragility precludes enhancement of the signal-to-noise of low abundance proteins through longer acquisitions. While protein identification and characterization relies to a great extent on the study of a set of accurate mass measurements derived from proteolytic digests, exact mass measurement of intact proteins still play an important role, especially in the study of post-translational modifications. Sinapinic acid (SA) is the matrix of choice for large proteins. However, the acquisition time and number of laser pulses must be extended in order to analyze low-abundant proteins or analytes that ionize inefficiently. SA crystals appear white and “fluffy” when properly spotted yet, these crystals are quickly depleted by laser irradiation during extended analysis, appearing as “flaking” of the matrix crystals. This laser-induced damage limits the number of scans that can be performed during an analysis. This limitation can impair analysis of low-abundance proteins, where averaging over a large number of scans enhances the signal-to-noise.

Alpha-cyano-4-hydroxycinnamic acid, which is mostly used for analysis of peptides, requires specific spotting techniques and the use of specific solvents to generate a homogeneous distribution of small crystals, which in turn produce the highest quality spectra. Alpha-cyano-4-hydroxycinnamic acid is also referred to herein as “alpha c”, “αC”, “alpha-cyano”, “α-cyano”, “CHCA” and “HCCA”.

Small matrix crystals spotted in a homogeneous thin film often provide the best results. It is reasonable to state that small crystals in a thin film provide the most analyzable surface area. Also, a thin film of small crystals eliminates the formation of pools of analyte mixed with solvent within large crystals. These pools of solvent could prevent association of analyte with matrix and therefore would not desorb upon laser irradiation.

There are published reports of dissolving matrix in acetone, which produces a thin film of small crystals. This method requires a wash to eliminate adduction by monovalent cations, and spotting needs to be performed in three steps (matrix spot, analyte spot, wash).

Target analyte:matrix crystals for MALDI are typically prepared by mixing and co-precipitating/co-crystallizing a matrix and an analyte, wherein the matrix, often an acidic aromatic matrix, is a molecule capable of absorbing light at wavelengths compatible with an emitting laser. When this matrix/analyte mixture is subject to laser, the matrix molecules absorb the laser energy and are desorbed from the target surface by vaporization, forcing the co-desorption of the analyte molecules with which they are co-crystallized. In the gas phase, the analyte molecules undergo ionization as they are ionized in a series of collision events, and accelerated into a time-of-flight tube where they are separated by their momentum.

In a MALDI-TOF MS method provided herein, a sample is prepared, mixed with a suitable matrix, and deposited on the MALDI target to form dry mixed crystals and, subsequently, placed in the source chamber of the mass spectrometer. Although the sample preparation and introduction into the source chamber can require a significant amount of time, protein identification by this technique has the advantage of short measuring time (typically, a few minutes) and negligible sample consumption (less than 1 pmol) together with additional information on microheterogeneity (e.g., glycosylation) and presence of byproducts.

The “dried-droplet” method of sample preparation is relatively simple. A saturated solution of matrix material is mixed with protein to a final concentration of 1-10 mM. A droplet (0.5-2 microliters) of the resulting mixture is placed on the mass spectrometer's sample stage. The droplet is dried at room temperature and, when the liquid has completely evaporated, the sample may be loaded into the mass spectrometer. Dried droplets are relatively stable and can be kept out of direct light and/or in vacuum for days.

The “slow crystallization” method may improve detection, particularly of larger proteins (Cohen et al., Anal Chem. 68:31, 1996; Botting, Rapid Commun Mass Spectrom. 14:2030, 2000). In this method, the protein sample is thoroughly mixed with the matrix solution and lety stand for a few hours. Large crystals, formed on the walls of the microfuge tube, are washed with water, scraped off, and applied to the MALDI target.

Polycrystalline thin films can be used in MALDI-TOF MS sample preparation. This method of sample preparation produces a uniform layer of very small crystals on the mass spectrometer's sample stage that are mechanically well adhered to the substrate. The crystals can be thoroughly washed without removing them from the surface. Li et al. (Dai et al., Anal Chem. 71:1087, 1999; Zhang et al., Anal Chem. 73:2968, 2001) developed a 2-layer sample preparation technique. This approach involves formation of a microcrystalline layer of matrix using fast solvent evaporation, followed by a deposition of a mixture of matrix and sample on top of the microcrystlline layer. The film grows rapidly, so it is not necessary to wait until the droplet is dry before washing the film, reducing effects caused by increasing contaminant concentrations as the droplet dries.

“Sandwich” MS sample preparation (Li et al., J. Am. Chem Soc. 118:11662, 1996; Kussman et al., J. Mass Spectrom. 32:593, 32, 1997) involves formation of a microcrystalline layer, followed by application of a sample (without matrix) and finally by depostion of another matrix layer. The sample is thus “sandwiched” between the two matrix layers.

### MALDI Matrix Additives

In some aspects, the invention is drawn to MALDI matrix additives or, more simply, matrix additives. Generally, matrix additives are included in matrices for the purpose of enhancing or adding a desirable characteristic to the matrix and/or matrix:analyte co-crystal.

A matrix additive of the invention, including without limitation a non-volatile matrix additive, can be mixed with MALDI matrix to initiate, promote, accelerate and/or support the formation of small crystals in thin layers in a single step, thus providing a large analyzable surface area for laser irradiation. The formation of small crystals in thin layers aided by the additive produce the most efficient desolvation of analyte molecules, thus maximizing the number of desorbable analyte molecules, which results in better ionization of the analyte molecules.

Additionally or alternatively, a matrix additive of the invention, including without limitation a non-volatile matrix additive, acts as an ion-exchanger, resulting in removal of monovalent cation contaminants.

Due to these and other properties or effects, a MALDI matrix additive of the invention results in improved signal-to-noise, reduction of monovalent cation-adducts, and suppressed matrix background noise.

As it is typically but not exclusively used in SA-based matrices, one type of formulation is referred to herein as a MALDI SA Matrix Additive. A specific but non-limiting of a MALDI SA Matrix Additive is Maxlon SA, which is described in more detail herein. MaxIon SA utilizes a novel diluent formulation that attenuates laser-induced damage of SA crystals and thus allows an increased number of MALDI acquisitions to be made and summed, thus enhancing the overall MALDI-MS spectral quality.

As it is typically but not exclusively used in CHCA-based matrices, one type of formulation is referred to herein as a MALDI CHCA Matrix Additive. A specific but non-limiting of a MALDI CHCA Matrix Additive is MaxIon AC, which is described in more detail herein. Maxlon AC includes one novel diluent based on a solubilizer (the zwitterionic surfactant NDSB) and silica resin as a MALDI matrix additive.

Silica is an efficient matrix crystal morphology modulator that enhances spectral quality even under high salt conditions. The silica resin additive promotes the formation of thin layers of small CHCA crystals thus reducing the matrix background, enhancing ionization of low-abundance species, and eliminating the suppressive effects of salt contamination.

### Protein Analysis Using MALDI MS

The invention provides methods and compositions for analyzing proteins. The invention can be used to detect, quantify, identify, characterize a protein. Some specific examples of analysis that the invention is suited for include, but are not limited to: (1) amino acid sequence determination and peptide mass fingerprinting (PMF), useful for identifying a gene encoding a protein of interest; (2) determination of sites of chemical modifications of proteins; (3) identification of ligand binding proteins and domains, useful in studies of interactions of protein with other proteins and molecules, and pharmacology/drug discovery.

In this and other aspects of the invention, the target molecule to be analyzed using mass spectrometry can be a hydrophilic molecule, e.g., a hydrophilic protein, such as a soluble protein or a small synthetic molecule, including oligonucleotides or peptides. Similarly, in this and other aspects of the invention, the target molecule can be a hydrophobic molecule, e.g., a hydrophobic protein, such as a membrane protein. Hydrophobic proteins of interest include ion channels or a transporters, particularly ligand-gated ion channels, such as a serotonin receptor, a gamma-aminobutyric acid receptor, a glycine receptor, a glutamategated chloride channel, a glutamate receptor, an ATP-gated channel, or an NMDA receptor. Other hydrophobic proteins of interest have at least one transmembrane domain and/or homology to the nicotinic acetylcholine receptor of Torpedo californica.

### Peptide Mass Fingerprinting (PMF)

One powerful use of mass spectrometers is to identify a protein from its peptide mass fingerprint and/or partial amino acid sequence. A peptide mass fingerprint is a compilation of the molecular weights of peptides generated by a specific protease. More recently, the ability to determine all or part of the amino acid sequence of the protein fragments. The sequence information, as well as data relating to the molecular weights of the parent protein prior to protease treatment and the subsequent proteolytic fragments is used to search genome databases for any similarly sized protein with identical or similar amino acid sequences and/or peptide mass maps

Proteases useful in PMF and amino acid sequencing include without limitation trypsin, chymotrypsin, elastase, Endoproteinase Arg-C, Endoproteinase Asp-N, Endoproteinase Glu-C, Endoproteinase Lys-C, Aminopeptidase M, Carboxypeptidase-Y and pronase. For details of protease cleavage reactions, see Sweeney, P. and Walker, J. M. Chapters 14-18 in: Enzymes of Molecular Biology, Methods in Molecular Biology 16, M. M. Burrell (ed.), Humana Press, Totowa, N.J. (1993). Chemical proteolysis (e.g., Edman degration) might also be used but are generally not preferred over enzymatic digestion, which can be targeted to specific amino acid sequences within a protein.

During a typical “in-gel” proteolysis protocol, a band comprising the protein of interest is isolated after electrophoresis. From this point, the protein might be further purified for sample preparation, but “in-gel” proteolysis, in which the protease is added to the partially purified protein, is generally preferred. In such cases, the enzymatic digest is performed in an aqueous solution where newly-generated hydrophobic fragments may irreversibly precipitate. The compositions and methods of the invention provide for “in-gel” proteolysis of hydrophobic proteins with a minimum amount of precipitation.

Several programs to assist with protease digestion analysis are available on the worldwide web. MS-Digest, for example, (available on the worldwide web at http://prospector.ucsf.edu/) allows for the in silico digestion of a protein sequence with a variety of proteolytic agents including trypsin, chymotrypsin, V8 protease, Lys-C, Arg-C, Asp-N, and CNBr. The program calculates the expected mass of fragments from these virtual digestions and allows the effects of protein modifications such as N-terminal acetylation, oxidation, and phosphorylation to be considered.

### Modifications of Proteins

The compositions and methods described herein can be used to study chemical and enzymatic modification and, in particular, to identify the sites (amino acid sequences) where the modifications occur. Post-translational modifications can be studied in this fashion. Also, proteins that are modified in signaling, and other cellular pathways, including apoptosis, can be studied. As a non-limiting example, proteins to which a phosphate group is added (by kinases) or removed (by phosphatases) often occur in cellular pathways. Examples of such studies include without limitation the following. Comparing peptide fingerprints before and after treatment with phosphatases indicates the position of modified amino acids in the protein's amino acid sequence directly, as has been shown for neurofilaments and EphB Receptors (Cleverley et al., Biochemistry 37:3917, 1998; Kalo et al., Biochemistry 38:14396, 1999). Utilizing this approach, residue-specific glycosidases can provide additional information about oligosaccharide side-chains in a protein, as has been shown for neurolin (Denzinger et al., J Mass Spectrom 34:435, 1999). The location and pairing of sulfides in a protein can be determined by reduction and proteolytic digest prior to MALDI analysis, as has been demonstrated for alpha-dendrotoxin (Belva et al., Rapid Commun Mass Spectrom. 14:224, 2000). Protein-modifying enzymes that can be used in the invention include without limitation kinases, phosphatases, glycosylases and deglycosylases. See, e.g., Jaquinod et al., Biol Chem 380:1307-1314 (1999); Kuster et al., Curr Opin Struct Biol. 8:393-400 (1998); Yan et al., Biochem Biophys Res Commun 259:271-282 (1999); and Nilsson, Mol Biotechnol 2:243-280 (1994).

### Ligand Binding Domains

MALDI-TOF MS can also be used to obtain information about quaternary structures, such as mapping of protein-protein contacts or ligand binding sites. For example, MALDI-TOF MS has been used to study the binding of alpha-neurotoxin to the nicotinic acetylcholine receptor and substance P to the neurokinin-1 tachykinin receptor, and the mapping of the agonist binding site of the cholecystokinin B receptor (Machold et al., Proc Natl Acad Sci USA 92:7282, 1995; Girault et al., Eur J Biochem 240:215, 1996; Anders et al., Biochemistry 38:6043, 1999).

Detectably-labeled ligands, such as known agonists, antagonists, receptor ligands, and derivatives thereof, are covalently linked to the binding site in a protein of interest in cross-linking reactions. Subsequent proteolysis and MALDI-TOF MS analysis result in PMF maps with additional peaks, indicating the cross-linking site on the protein.

Photoaffinity labeling represents one type of cross-linking strategy that can be used to identify and characterize those regions of a protein in which an interaction with low-molecular-mass ligands takes place. In the specific instance of nAChR, photoaffinity probes include without limitation [3H]4-Benzoylbenzoylcholine (Wang et al., J. Biol. Chem., 275:28666, 2000), [3H]nicotine (Middleton et al., Biochemistry 30:6987, 1991) and p-(N,N-dimethyl)aminobenzenediazonium fluoroborate (Galzi et al., J. Biol. Chem. 256:10430, 1990). Ligand-binding regions of glycoprotein P have also been studied using photoaffinity labeling and MALDI-TOF MS (Ecker et al., Mol Pharmacol. 61:637, 2002).

### Hydrophobic Proteins

The invention provides compositions and methods for studying, for example, hydrophobic proteins, including without limitation membrane proteins. Proteins that span a biological membrane are said to have one or more transmembrane domains (TMDs). Membrane proteins may represent as much as one half of the total diversity of some proteomes, and play many roles in fundamental biological processes such as cell-cell interactions, cell signaling, protein trafficking, ion and solute transport and intracellular compartmentalization. Many pharmacological targets are membrane proteins. Membrane and other hydrophobic proteins are notoriously difficult to study with conventional methods.

Membrane protein can be from any biological source membrane, including without limit cellular membranes, viral envelopes (Kim et al., Anal Chem. 73:1544, 2001) and membranes from an organelle (such as a nucleus, a nucleolus, a mitochondrion, a chloroplast, and the endoplasmic reticulum). In the case of mitochondria and chloroplasts, both the inner and outer membranes, and the intermembrane space, can be sources of membrane and other hydrophobic proteins.

The invention can be applied to any membrane protein, including but not limited to the following exemplary receptors and membrane proteins. The proteins include but are not limited to receptors (e.g., G-protein coupled receptors, or GPCRs, sphingolipid receptors, neurotransmitter receptors, sensory receptors, growth factor receptors, hormone receptors, chemokine receptors, cytokine receptors, immunological receptors, and compliment receptors, FC receptors), channels (e.g., potassium channels, sodium channels, calcium channels.), pores (e.g., nuclear pore proteins, water channels), ion and other pumps (e.g., calcium pumps, proton pumps), exchangers (e.g., sodium/potassium exchangers, sodium/hydrogen exchangers, potassium/hydrogen exchangers), electron transport proteins (e.g., cytochrome oxidase), enzymes and kinases (e.g., protein kinases, ATPases, GTPases, phosphatases, proteases.), cyytochrome P450 enzymes, structural/linker proteins (e.g., Caveolins, clathrin), adapter proteins (e.g., TRAD, TRAP, FAN), chemotactic/adhesion proteins (e.g., ICAM11, selectins, CD34, VCAM-1, LFA-1, VLA-1), and phospholipases such as PI-specific PLC and other phospholipases.

### Ligand-Gated Ion Channels (LGICs)

In particular, the invention can be applied to proteins or domains that are homologous to nAChR and/or domains thereof, and other ligand-gated ion channels (LGICs). LGICs include a serotonin (5-hydroxytryptamine or 5-HT) receptor (e.g., 5-HT3A and 5-HT3B); a gamma-aminobutyric acid receptor; a glycine receptor; a glutamate-gated chloride channel; a glutamate receptor; an ATP-gated channel; and an NMDA receptor.

Although it was once thought that all LGICs belonged to a single superfamily of channels, there may in fact be three distinct superfamilies:

(1) Members of the cys-loop superfamily contain four membrane-spanning segments without any pore loops. The term “Cys-loop” refers to the presence of a pair of disulphide-bonded cysteines near the N-terminal of the protein. In mammals, the superfamily of Cys loop LGICs is assembled from a pool of more than 40 homologous subunits. These subunits have been classified into four families representing channels that are gated by acetylcholine, serotonin, gamma-aminobutyric acid, or glycine.

The muscle-type nicotinic acetylcholine (ACh) receptor (mnAChR) is an exemplary cys-loop protein. The basic structural features of mnAChRs (four membrane-spanning segments, ligand-binding sites at subunit interfaces and a pore formed by M2) are thought to be preserved in the other members of the cys-loop superfamily. Two members of the cys-loop superfamily, gamma-aminobutyric acid type A receptors (GABAAR) and glycine receptors (GlyR), are permeable to anions rather than cations. Neuronal nicotinic ACh receptors (nnAChR) include without limitation nAchR.

(2) The ionotropic glutamate receptor (GluR) superfamily consists of three families, all of which are activated in vivo by L-glutamate. The three families are distinguished by their affinity for the synthetic agonists-amino-5-methyl-3-hydroxy-4-isoxazole propionic acid (AMPA), N-methyl-D-aspartame (NMDA) and kainate.

(3) The ionotropic, purinergic receptor (P2X) ATP-activated superfamily of LGICs exhibit two membrane-spanning regions and no pore-loops. All of the receptors are about equally permeable to Na⁺ and K⁺ and also have significant Ca⁺⁺ permeability.

Volatile anaesthetics and alcohols [e.g., soflurane and butanolanaesthetics (ether, cyclopropane, butane)] have both inhibitory and potentiating effects on mnAChRs (McLarnon J G, Pennefather P, Quastel D M J. Mechanisms of nicotinic channel blockade by anesthetics. In: Roth S H, Miller K W, eds. Molecular and Cellular Mechanisms of Anesthetics. New York: Plenum Press, 1986; 155-164).

Some structural information about the ligand-binding domains of LGICs has been obtained from crystallographic studies. The ligand-binding domain of GluR has been imaged at 1.9 Å resolution to reveal a clamshell-shaped shape having two lobes surrounding a large binding cleft. An Ach-binding protein isolated from snail glial cells has an amino acid sequence that resembles the extracellular portion of members of the cys-loop superfamily, and like the mammalian channels, it forms a pentamer. Unlike the ligand-binding domain of GIuR, however, there is no large binding cleft in the ACh-binding protein; rather, ligand-binding sites are formed at the interface between each pair of subunits.

### Related Proteins

The methods of the invention can be applied to known proteins, domains and/or amino acid sequences, or to uncharacterized proteins, domains and/or amino acid sequences that are substantially identical and/or have homology to each other.

### Identity

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% or about 60% identity, optionally 65% or about 65%, 70% or about 70%, 75% or about 75%, 80% or about 80%, 85% or about 85%, 90% or about 90%, or 95% or about 95% identity over a specified region), when compared and aligned for maximum correspondence over a comparison window or designated region, as measured using sequence comparison algorithms or by manual alignment and visual inspection. Such sequences are then said to be “substantially identical.” Preferably, the identity exists over a comparison window or designated region that is at least 25 or about 25 nucleotides (nt) or amino acids (aa) in length, more preferably over a region that is from 25 or about 25 to 75 or about 75 nt or aa in length, even more preferably from about 75 or 75 to 150 or about 150, or more, nt or aa in length.

By a polypeptide having an amino acid sequence at least, for example, 95% “identical” to a reference amino acid sequence it is intended that the amino acid sequence of the polypeptide is identical to the reference sequence, except that the polypeptide sequence may include up to five amino acid alterations, including deletions and insertions, per each 100 amino acids of the reference amino acid sequence. In other words, to obtain a polypeptide having an amino acid sequence at least 95% identical to a reference amino acid sequence, up to 5% of the amino acid residues in the reference sequence may be deleted or substituted with another amino acid, and/or a number of amino acids up to 5% of the total amino acid residues in the reference sequence may be inserted into the reference sequence.

For sequence comparison, typically a known sequence acts as a reference sequence, to which test sequences are compared. A “comparison window”, as used herein, includes reference to a segment of any one of the number of contiguous positions selected from the group consisting of from 20 or about 20 to 600 or about 600, usually from 50 or about 50 to 200 or about 200, more usually 100 or about 100 to 150 or about 150, in which a sequence may be compared to a reference sequence of the same number of contiguous positions after the two sequences are optimally aligned. Methods of alignment of sequences for comparison include the following. Alignment of sequences for comparison can be conducted, e.g., by the local homology algorithm of Smith & Waterman, Adv. Appl. Math., 2:482 (1981), by the homology alignment algorithm of Needleman & Wunsch, J. Mol Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement).

### Homology

The overall homology of a protein to another protein, or of a domain or motif to another, can be 50% or about 50%, 60% or about 60%, 70% or about 70%, 75% or about 75%, 80% or about 80%, 85% or about 85%, 90% or about 90%, 95% or about 95% or 99% or about 99%. The percent homology between two sequences is determined using sequence analysis software. Such software matches similar sequences by assigning degrees of homology to various insertions, deletions, substitutions, and other modifications. Exemplary software include: The Sequence Analysis Software Package of the Genetics Computer Group, University of Wisconsin Biotechnology Center, Madison, Wis; (Devereux et al., Nucleic Acids Res. 12:387, 1984); The algorithm of E. Myers and W. Miller (CABIOS, 4:11, 1989), which has been incorporated into the ALIGN program (version 2.0), using a PAM120 weight residue table, a gap length penalty of 12 and a gap penalty of 4; The NBLAST and XBLAST programs (version 2.0) of Altschul et al. (J. Mol. Biol. 215:403, 1990).

BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (Nucleic Acids Res. 25:3389, 1997). When utilizing BLAST and gapped BLAST programs, the default parameters of the respective programs (e.g., XBLAST and NBLAST) can be used.

A number of sequence databases can be searched for homologous molecules, including, for example, the GenBank database (National Center for Biotechnology Information, Bethesda), EMBL data library (European Bioinformatics Institute, Cambridge, UK), the Protein Sequence Database and PIR-International, and SWISS-PROT. The ExPASy (Expert Protein Analysis System) proteomics server of the Swiss Institute of Bioinformatics (SIB), available on the worldwide web at http://www.expasy.ch/, provides information on, and URLs (links) for numerous available databases and software tools for the analysis of protein sequences.

It should be appreciated that the present invention is applicable to any sequence databases and analysis tools available to the skilled artisan and is not limited to the examples described herein.

### III. Kits

The invention also provides kits that include one or more compositions of the invention. For example, a kit can comprise containers consisting of one, two or more compounds or mixtures thereof in either a solution or in powdered (dessicated or lyophilized) form. Such compounds and mixtures include the MS-compatible solubilizing formulations described herein, and/or the non-volatile MS matrix additives, and optionally several dilutions thereof. A kit can optionally further comprise one or more other kit components, including but not limited to one or more chaotropes, optionally mixed in an optimized proportion; one or more MALDI matrices; one or more buffers; one or more standard or control proteins and/or one or more MS calibrants. Containers are typically sealed and can be, e.g., a packet, a bag, a vial, a tube, a blister pack, a microtiter plate or any other suitable container.

In one aspect, the invention is drawn to kits. In one embodiment of this aspect of the invention, a kit of the invention comprises one or more MS-compatible solubilizers. The MS-compatible solubilizer can be one described herein. By way of non-limiting example, the MS-compatible solubilizer comprises a compound selected from the group consisting of ASB-C8Ø, Octyl-beta-D-1-thioglucopyranoside, n-Dodecanoylsucrose, SB14 and a non-detergent sulfobetaine. The MS-compatible solubilizerin a kit is typically provided in the form of a concentrated stock solution, e.g., a 1.5×, 2×, 3×, 4×, 5×, 10×, 15×, 25×, 50× or 100× stock solution.

Optionally, the kit further comprises one or more matrix compositions. Non-limiting examples of matrix compositions include sinapinic acid and alpha-cyano-4-hydroxycinnamic acid. Optionally, the kit further comprises one or more matrix solvents. Non-limiting examples of matrix solvents inlcude 0.1% trifluoroacetic acid and 100% acetonitrile.

Optionally, the kit further comprises one or more chaotropes. Non-limiting examples of chaotropes include urea, thiourea and guanidine chloride.

Optionally, the kit further comprises one or more enzymes, such as a protease. Non-limiting examples of proteases include TEV protease, trypsin, chymotrypsin, elastase, Endoproteinase Arg-C, Endoproteinase Asp-N, Endoproteinase Glu-C, Endoproteinase Lys-C, Aminopeptidase M, Carboxypeptidase-Y and pronase.

Optionally, the kit further comprises one or more buffers; one or more cross-linkers; one or more standards, controls or calibrants; and a product manual that describes storage conditions and one or more experimental protocols. Non-limiting examples of experimental protocols inlcude a protocol for direct analysis and calibration of intact hydrophobic proteins, a buffer exchange protocol, and a trypsin digestion protocol.

In one embodiment, a kit of the invention comprises (a) a container comprising a solution of ASB-C8Ø, Octyl-beta-D-1-thioglucopyranoside, n-Dodecanoylsucrose and SB14; (b) a container comprising NDSB-201; and, optionally, one or more of: (c) a container comprising one or more molecular weight standards; (d) a container comprising sinapinic acid; (e) a container comprising alpha-cyano-4-hydroxycinnamic acid; (f) a container comprising trifluoroacetic acid; and (g) a container comprising acetonitrile. In a more specific embodiment, a kit of the invention comprises (a) a container comprising 10 ml of a solution of ASB-C8Ø at 125 mM, Octyl-beta-D-1-thioglucopyranoside at 50 mM, n-Dodecanoylsucrose at 3.8 mM, and SB14 at 1 mM; (b) a container comprising 25 ml of 500 mM NDSB-201; and, optionally, one or more of: c) a container comprising 25 μL of 90 kDa InvitroMass protein standard; (d) a container comprising 20 mg of sinapinic acid; (e) a container comprising 20 mg of alpha-cyano-4-hydroxycinnamic acid; (f) a container comprising 20 ml of 0.1% trifluoroacetic acid; and (g) a container comprising 1 ml of 100% acetonitrile.

Another kit can include (a) a container comprising a solution of NDSB 201, NDSB 256, and SB14; and (b) a container comprising a protein standard. In a more specific embodiment, a kit of the invention comprises (a) a container comprising 5 ml of a solubilizer solution of 125 mM NDSB 201, 125 mM NDSB 256, and 1.1 mM SB14 in 125 mM Ammonium bicarbonate, pH 7.8; and (b) a container comprising 50 microliters of a standard tryptic digest of BSA in 1× solubilizer.

Instructions may also be included in a kit. Typically, sufficient documentation will be included to describe the application of this kit's components to the direct analysis and calibration of intact hydrophobic proteins; general recommendations for contaminants, particularly SDS, Triton X, CHAPS, and HEPES; and a description of performance and analysis of tryptic digests supported by the solubilizers.

A kit may also comprise one or more solid supports, which can optionally be coated with one or more one or more MS-compatible compositions of the invention. Such solid supports include without limitation beads, porous beads, crushed particles, membranes, tubing and planar surfaces (e.g., plates). Descriptions of representative kits of the invention are given in the Examples herein.

All patents, patent publications, patent applications and other published and world wide web based references mentioned herein are hereby incorporated by reference in their entirety as if each had been individually and specifically incorporated by reference herein.

Headings found herein are for the convenience of the reader and do not limit the invention in any way.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Analysis of Detergents and Other Surfactants

This Example illustrates testing of detergents and other surfactants to identify solubilizer formulations that can be used for methods of the present invention that involve MALDI-TOF-MS analysis. The formulations include surfactant molecules that have been independently tested for suppression effects on the ionization of peptides and intact proteins by MALDI.

A MALDI-MS compatible surfactant blend formulation was devised by separately assaying the effect of individual components on the ionization efficiency of a peptide mixture. The performance of BLEND I in MALDI-TOF MS was tested using beta-galactosidase (b-gal) and BSA. Bovine serum albumin (BSA), a commonly utilized test protein, was used as an exemplary intact protein, and a tryptic digest of b-galactosidase (t-b-gal) was used as an exemplary peptide mixture. Like BSA, b-gal is a commonly utilized test protein; moreover, the b-gal tryptic fragments represent a range of solubility from hydrophilic to hydrophobic.

MALDI-MS analysis of t-beta-gal in a solution containing the test surfactant was carried out separately for each surfactant. Table 4 lists the various solutions of t-beta-gal and detergent that were examined. Each detergent was tested at a concentration near its CMC, the exception being ASB-C8Ø, for which there is no CMC data. Samples were run in parallel with an equivalent control sample of t-beta-gal containing no surfactant.

MALDI-MS analysis revealed that most components (with the exception of SB10) do not significantly suppress ionization, since total ion counts were similar for the test samples and the control sample. At the concentrations tested, n-Dodecanoylsucrose suppressed t-beta-gal peptides the least, and SB10 suppressed the most. In the (m/z 1199-3180) data range, n-Dodecanoylsucrose matched 100% of the mass-ions identified in the t-beta-gal sample without surfactant (MASCOT® score 133, 14% sequence coverage).

TABLE 4
MALDI-TOF MS TESTING OF INDIVIDUAL SURFACTANTS

| | | Test | % Match |
|---|---|---|---|
| Surfactant | CMC | Concentration | (m/z 1199-3181) |
| ASB-C8Ø* | unknown | 0.025 mM | 94% |
| Octyl-beta-D-1- | 9 mM | 9.25 mM | 71% |
| thioglucopyranoside | | | |
| n-Dodecanoylsucrose | 0.3 mM | 0.38 mM | 100% |
| SB10** | 25-40 mM | 2.9 mM | 58% |
| SB14*** | 2-4 mM | 0.96 mM | 71% |

*ASB-C8Ø (a.k.a. ASB-C8F) is 4-n-Octylbenzoylamido-propyl-dimethylammonio sulfobetaine.
**SB10 (a.k.a. sulfobetaine 10) is n-Decyl-N,N-dimethyl-c-ammonio-1-porpanesulfonate.
***SB14 (a.k.a. sulfobetaine 14) is n-Tetradecyl-N,N-dimethy-3-ammonio-1-propanesulfonate.

Table 4 also lists the percentage of match for each individual surfactant for the mlz 1199-3180 data range. The MALDI-MS data was analyzed further to identify the t-beta-gal fragment that corresponded to individual mass-ions that had been selectively suppressed in the presence of surfactant. Of the 12 suppressed peptides, 8 contained one or more tryptophan residues, and 10 contained two or more aromatic residues. The mechanism of this suppression is not known and does not appear to be surfactant specific, although the selective suppression effect was most pronounced with SB14.

Based on the results of the preceding experiments, a new formulation was devised (Table 5). This formulation, called “BLEND I” herein, keeps each component above its CMC and excludes SB10 due to its suppressive nature (SB10 is commercially available from, e.g., A.G. Scientific, San Diego, Calif. or USB, Cleveland, Ohio).

TABLE 5
FORMULATION OF 5X BLEND I IN DEIONIZED WATER

| Detergent | Concentration | Supplier |
|---|---|---|
| ASB-C8Ø | 0.125 mM | CalBiochem (a) |
| Octyl-beta-D-1-thioglucopyranoside | 50 mM | Sigma (b) |
| n-Dodecanoylsucrose* | 3.8 mM | CalBiochem (a) |
| SB14 | 1 mM | Fluka (c) |

*a.k.a. b-D-Fructo-furanosylsucrose Monolaurate n-Monododecanoate-a-D-glucopyranoside.
(a) Calbiochem ® is a brand of EMD Biosciences, Inc. (San Diego, CA).
(b) Sigma-Aldrich Corp. (St. Louis, MO).
(c) Fluka is a brand of Sigma-Aldrich Corp. (St. Louis, MO).

There are no data regarding the CMC of ASB-C8Ø, but MALDI-MS spectra of samples using other formulations often show strong peaks of the dimer form of ASB-C8Ø (˜881 Da). The concentration of ASB-C8Ø in BLEND I was determined by simply diluting the surfactant concentration in a t-beta-gal sample until the dimer peak was no longer dominant.

In order to test the characteristics of BLEND I, samples (400 fmol) of t-b-gal were prepared in 1× BLEND I. These samples were then analyzed by MALDI-MS. The MALDI-MS spectra demonstrate that t-b-gal peptides ionize with the same efficiency in BLEND I as in 50% acetonitrile. The MALDI spectra further demonstrate that a positive identification of the t-b-gal tryptic map fingerprint can be made even at 4 fmol (MASCOT® score 139, 21% sequence coverage). Results from experiments using 350 fmol and 35 fmol of BSA demonstrate that BSA ionizes with nearly the same efficiency and sensitivity whether in water or BLEND I. Thus, the BLEND I ionization suppression effects by MALDI-MS are insignificant.

### Example 2

### Analysis of Cytochrome P450 1A2

The preceding experiments show that BLEND I does not interfere with ionization and sensitivity of the MALDI-MS analysis of peptides and proteins. However, for some applications, especially those involving hydrophobic target molecules, the surfactant blend must be an efficient solubilization agent. Thus, the performance characteristics of BLEND I were tested as follows.

Drop-dialysis on Cytochrome P450 1 A2 (Invitrogen/PanVera) was carried out in order to exchange the 20% glycerol included in the stock storage buffer for BLEND I. Cytochrome P450 was selected as the test protein because it contains a transmembrane segment within the first 30 N-terminal residues and thus requires a surfactant to be soluble in an aqueous solution. Drop-dialysis was performed using a 25 mm filter-membrane (Millipore) placed on top of an inverted 15 mL conical tube cap containing 3 ml of 0.05× BLEND I. Three (3) μL of Cytochrome P450 (1.7 μg/μL) was mixed with 3 μL of 1× BLEND I and incubated at RT for 10 min. The 6 [L drop was then placed on the membrane and dialysed for 2 hrs, followed by a bottom buffer change and an additional 2 hrs. During this process, detergent monomers, salts and glycerol are allowed to freely equilibrate between the drop and the bottom buffer. Detergent micelles and protein are retained at the drop due to the ˜2,500 Da molecular weight cut-off of the filter.

At the end of the dialysis, it is estimated that the sample contains 8×10⁻⁵% glycerol. It should be noted that the increase from the relatively high concentration of glycerol caused the volume of the drop to increase three to five fold (swelling often occurs at concentrations of glycerol >20%). The MALDI-MS spectra of Cytochrome P450 prior to and after dialysis show that 20% glycerol suppresses the ionization of Cytochrome P450, while exchange for BLEND I relieves this suppression. The negative control (water) resulted in loss of protein.

Although 20% glycerol can maintain some hydrophobic proteins in aqueous solution, it does not form micelles. In order to test whether BLEND I could be utilized to exchange a micelle-forming surfactant such as Triton X100, a commonly used detergent in the extraction and purification of hydrophobic proteins, the following experiments were carried out. A solution of Cytochrome P450 (500 ng/ml) in 0.5% Triton X100, 6% glycerol was prepared for drop dialysis. The solution was mixed 1:1 (v/v) with 5× BLEND I and incubated for 10 min at RT. The solution was subsequently dialyzed as described above. The MALDI-MS spectra for Cytochrome P450 in 0.5% Triton X100 prior to and after dialysis show that the ionization of Cytochrome P450 is completely suppressed whereas dialysis against BLEND I allows ionization to be restored.

Detergent exchange may be performed in the presence of the 90 kDa InvitroMass calibrant. Besides providing an internal mass standard (alternatively the standard can be spiked into the analyte solution immediately prior to MALDI-MS analysis), the inclusion of the standard is a positive control for testing the possible residual presence of interfering detergent.

### Example 3

### Enhanced Sequence Coverage of the Peptide Mass Fingerprint for Cytochrome P450 1A2

During a typical “in-gel” proteolysis protocol, the enzymatic digest is performed in an aqueous solution where hydrophobic fragments may irreversibly precipitate. The application of BLEND I during “in-gel” proteolysis was tested as follows.

A sample containing 75 pmol of the membrane protein Cytochrome P450 1A2 (Invitrogen/PanVera) was prepared for gel electrophoresis in the standard manner and separated by SDS-PAGE. A band at ˜60 kDa corresponding to P450 was excised and destained with 50% acetonitrile/25 mM ammonium bicarbonate pH 8.0. Two hundred (200) μL of 100% acetonitrile was added to the gel piece and then dried down using a speed-vac apparatus. The sample was then rehydrated in a 10 ng/μL solution of trypsin in 25 mM ammonium bicarbonate pH 8.0 plus 1× BLEND I and incubated overnight at 37° C. After proteolysis, the digested peptides were extracted using one 10 μL 2.5% TFA wash, and an additional 10 μL 25% acetonitrile/2.5% TFA wash. The extracted samples were analyzed by MALDI-MS and the resulting mass/ions identified by the MASCOT® software (Matrix Science, London, UK). The addition of BLEND I extended the sequence coverage from 40% (without BLEND I) to 48% (with BLEND I).

### Example 4

### Enhanced Sequence Coverage of the Peptide Mass Fingerprint for the Nicotinic Acetylcholine Receptor

The nicotinic acetylcholine receptor (nAChR) was extracted from the electric organ of Torpedo californica and affinity purified as previously described (DaCosta et al., J. Biol. Chem. 277:201, 2002). Purified material was concentrated by ultracentrifugation where the pelleted phospholipid vesicles with nAChR were resuspended in SDS-PAGE loading buffer (Invitrogen). Approximately 2 μg of nAChR was run per lane of a NuPAGE SDS-PAGE gel (4-12%) (Invitrogen). Bands were detected by Simply Blue staining (Invitrogen).

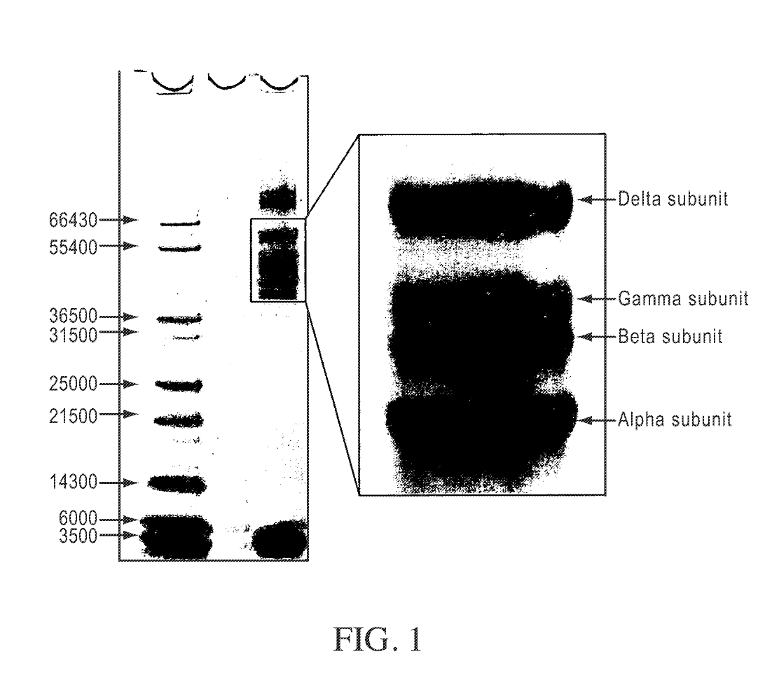

FIG. 1 shows the results of SDS-PAGE of purified T. californica nAChR. The electrophoretic migration pattern of the individual subunits (alpha, beta, gamma and delta) that assemble into the native, pharmacologically active nAChR is shown in relation to molecular weight markers. The inset to the right more specifically identifies each subunit of the nAChR.

Bands were excised manually and destained in 50% acetonitrile/25 mM ammonium bicarbonate (ABC) at pH 7.8 (ABC-7.8) or 8.0 (ABC-8.0) until blue color was no longer visible. Bands were dehydrated with 200 μL of 100% acetonitrile and vacuum centrifuged to dryness.

The gel pieces were then rehydrated on ice with a 10 ng/μL solution of trypsin (Promega) in 25mM ABC-7.8 or ABC-8.0 and 1× BLEND I to a final concentration of 10 mM n-octyl-b-thioglupyranoside and 200 mM Zwittergent 3-14. Proteolysis was allowed to proceed overnight at 37° C. Peptides were extracted with 50% acetonitrile/2.5% TFA.

MALDI-MS analysis was conducted on a ABI DE-STR MALDI-TOF (laser settings set 1650-1850, 250 nsec delay). MS/MS of select mass-ions was performed on a 4700 Voyager MALDI-TOF-TOF(MS/MS laser setting:4000, CID gas off, 250 nsec delay) (Applied Biosystems). All analyses were conducted in reflectron mode. Samples were spotted on a stainless steel MALDI target using a “sandwich” spotting method wherein the analyte is spotted between two layers of alpha-cyano-4-hydroxycinnamic acid (Fluka). Assignments of the MS data to the nAChR sequence were performed using MASCOT® software.

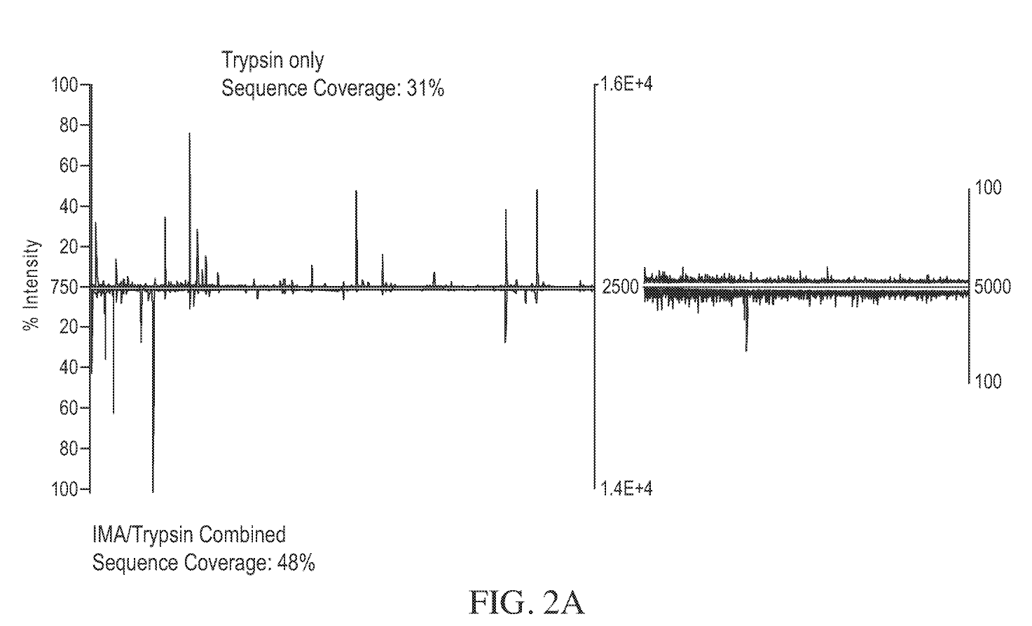

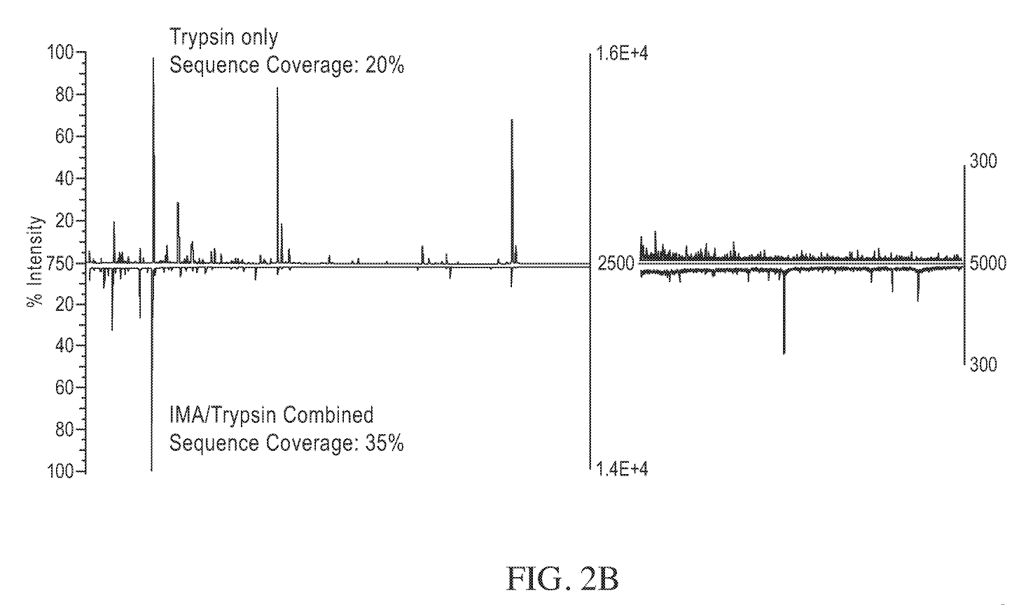

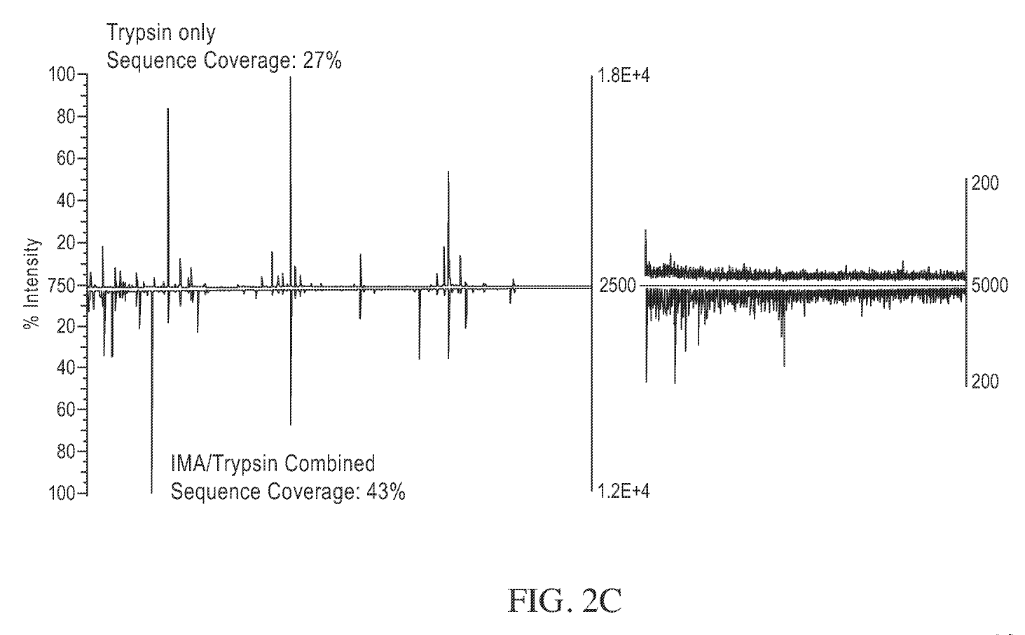

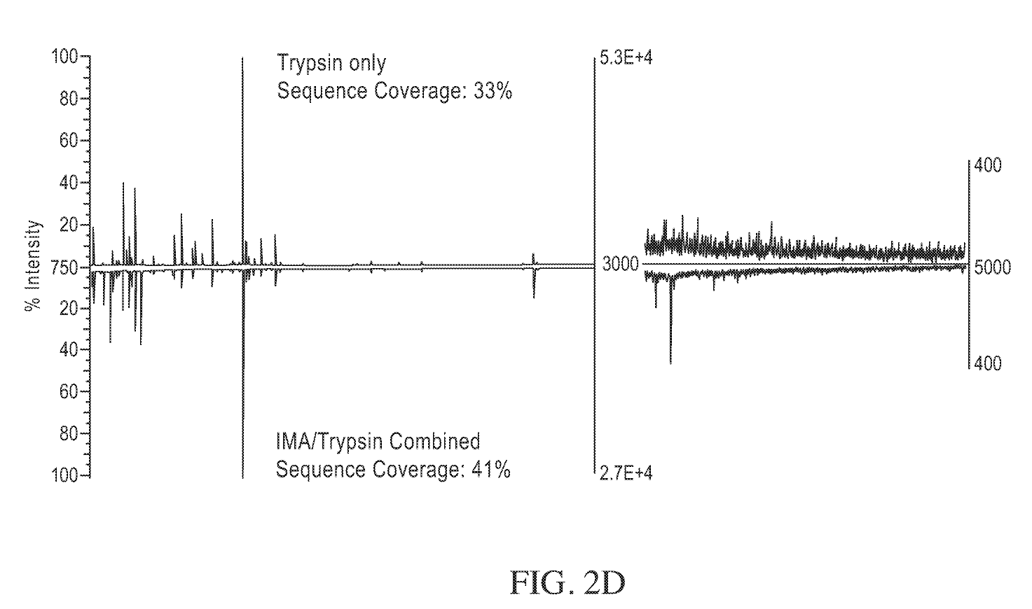

FIG. 2 shows the MALDI-MS spectra for the peptide mass fingerprint of the delta (A), gamma (B), beta (C) and alpha (D) subunits of nAChR. The blue spectra represent the digests performed in the absence of BLEND 1, whereas the red spectra represent the digests performed in the presence of 1× BLEND I. The sequence coverage without BLEND I is shown above the spectra, whereas the combined sequence coverage of the digests with and without BLEND I is shown above the respective nAChR subunit sequence. These results are summarized in Table 6.

In performing parallel processing of nAChR proteins, in which one process included BLEND I surfactant mix in the trypsin digest of the protein, and the alternate process omitted surfactants in the trypsin digest. Each treatment generated unique peptides, with the peptides generated from each process had overlapping sequences. Because of this, combining the peptide pools from each separation protocol enhanced the sequence coverage of the protein. On average, combining the data from the two sample preparations (digestion with Blend I and digestion without Blend I) yielded 1.5 times the sequence coverage for the nAChR than conventional “in-gel” or solutions proteolysis protocols.

TABLE 6
EXTENT OF SEQUENCE COVERAGE
WITH OR WITHOUT BLEND I

| NAChR | Trypsin only Sequence | BLEND I/Trypsin Combined |
|---|---|---|
| subunit | Coverage | Sequence Coverage: |
| Alpha | 33% | 41% |
| Beta | 27% | 43% |
| Gamma | 20% | 41% |
| Delta | 31% | 48% |

### Example 5

### Identification of Pharmacologically Relevant nAChR Regions

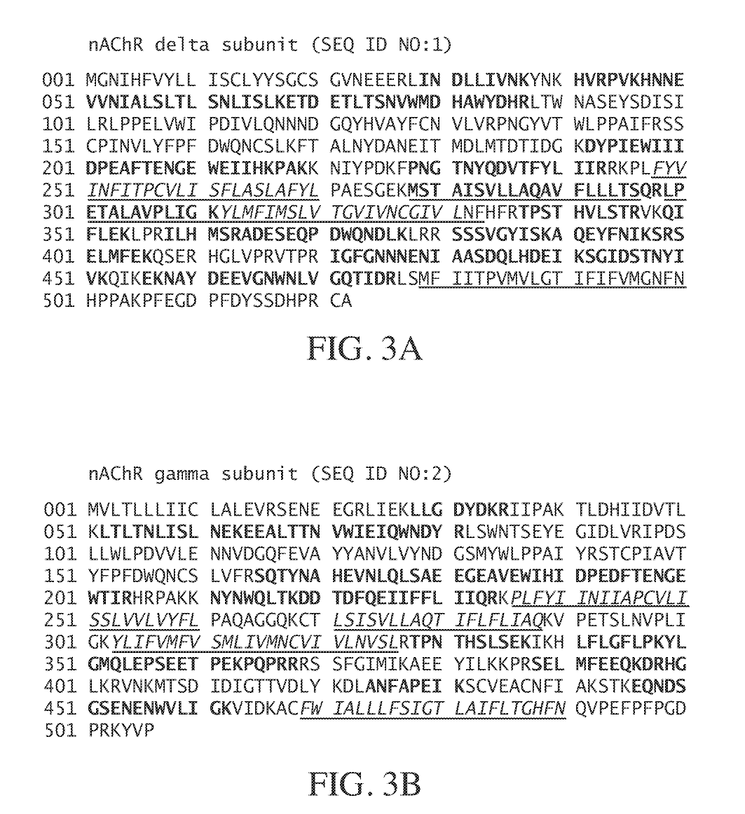

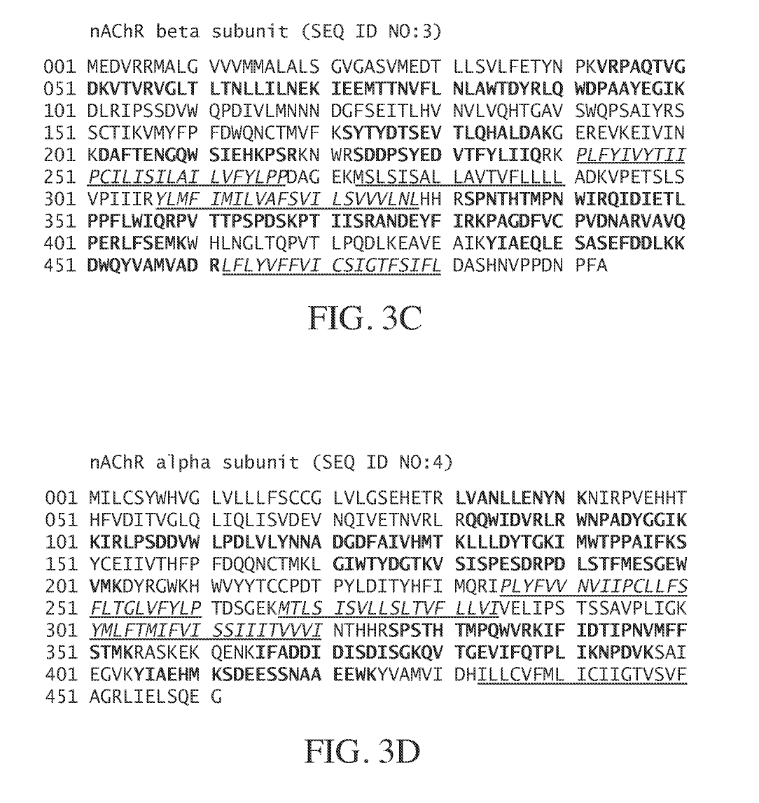

FIG. 3 shows the sequences of the delta (A), gamma (B), beta (C) and alpha (D) subunits of nAChR. Amino acid sequences of transmembrane domains are underlined, and the amino acid residues of the peptide fragments identified by these experiments are shown in bold (SEQ ID NOS:5-7).

The amino acid sequences identified in the previous Example were examined and evaluated for their pharmacological relevance. An illustration of the Lymnae stagnalis acetylcholine binding protein crystal structure (Brejc et al., Nature 411:269, 2001) was superimposed over the electron micrograph image of the Torpedo marmorata nAChR transmembrane domain (Miyazawa et al., Nature 423:949, 2003). Images were generated using the coordinates posted on Protein Databank and PDP viewer software. The 3-dimensional locations of amino acid sequences identified in the previous Example were determined and compared to regions of known pharmacological relevance. Table 7 summarizes the results.

Opening up hydrophobic regions for MALDI-TOF MS is expected to reveal other pharmacologically relevant regions in areas that are normally unavailable for MALDI-TOF MS. Novel pharmacologically relevant regions may be found in previously inaccessible domains, including transmembrane and intramembranous domains. For example, Labrou et al. (J. Biol. Chem. 276:37944, 2001) suggest that, in the case of the Tachykinin NK2 receptor, its peptide binding site is at least in part formed by residues buried deep within the transmembrane bundle, and that this intramembranous binding domain may correspond to the binding sites for substantially smaller ligands. The Tachykinin NK2 receptor thus has sites where ligands bind, which are expected to be pharmacologically relevant, and these sites are in hydrophobic regions, which are the types of regions towards which the solubilizing agents of invention are directed.

TABLE 7
IDENTIFIED PHARMACOLOGICALLY RELEVANT
NACHR REGIONS

| | | Amino | | |
|---|---|---|---|---|
| | | Acid | | |
| m/z | Subunit | Residues | Region | Significance |
| 2262.4 | Delta | 278-297 | M2 | Pore forming alpha- |
| | | | | helical rod; target |
| | | | | of channel blockers |
| | | | | and noncompetitive |
| | | | | antagonists (Labarca et |
| | | | | al., Nature 376: 514, |
| | | | | 1995) |
| 1321.8 | Delta | 299-311 | M2-M3 loop | Conformational linker |
| | | | SEQ ID NO: 5 | between N-terminal |
| | | | | domain and pore domain |
| | | | | (Grosman et al., J Gen |
| | | | | Phys 116: 327, 2000; |
| | | | | Miyazawa et al., |
| | | | | Nature 423: 949, |
| | | | | 2003). |
| 1153.6 | Alpha | 170-179 | Ligand domain | Site of ligand, |
| | | | SEQ ID NO: 6 | modulator binding |
| | | | | (Lester et al., |
| | | | | TINS 27: 29, 2004; |
| 2727.3 | Alpha | 180-203 | Ligand domain | Leite et al., PNAS |
| | | | SEQ ID NO: 7 | USA 100: 1304, 2003; |
| | | | | Brejc, et al., Nature |
| | | | | 411: 269, 2001). |

### Example 6

### Further Surfactant/Detergent Analysis (NDSB-201)

An additional MALDI-MS compatible surfactant that may be used as an alternative to or in combination with BLEND I is 250 mM NDSB-201 (a.k.a. Invitrogen MS-compatible solubilizer “B”, Invitrosol B or IMB). This NDSB (non-detergent sulfobetaine) compound has been used as to prevent aggregation of hydrophobic proteins in aqueous solution or enhance the renaturation of proteins from insoluble inclusion bodies.

Pure bovine serum albumin (BSA) (Sigma-Aldrich, St. Louis, Mo., USA) was diluted in ABC-7.8 (200 mM ammonium bicarbonate, pH 7.8) to a 1.5 mM working solution. Sequencing grade trypsin (Promega, Madison, Wis., USA) was added to the solution at 1:100 enzyme-to-substrate ratio (w/w). Half the sample was mixed 1:1 (v/v) with deionized water (negative control) (Pierce Biotechnology, Rockford, Ill.), and the other half was mixed 1:1 (v/v) with 500 mM NDSB-201 (Calbiochem®, a brand of EMD Biosciences, Inc., San Diego, Calif.). Both samples were then incubated at 37° C. overnight.

MALDI-TOF-MS analysis was performed using an Applied Biosystems Voyager DE STR instrument. Samples (0.5 μL) were spotted on the MALDI target using a “sandwich method” wherein 0.5 μL of alpha-cyano-4-hydroxycinnamic acid (Sigma-Aldrich, St. Louis, Mo., USA) [7 mg/ml in 50% acetonitrile (Pierce)] with 0.1% TFA (Pierce) was first spotted on the plate and allowed to dry. An aliquot (0.5 μL) of sample was then spotted as an over-layer and allowed to dry. Finally, an additional 0.5 μL spot of matrix was applied to re-wet the lower two layers and the slurry was permitted to air dry once again. All MALDI-TOF-MS spectra were acquired in the positive reflectron mode (unless specified) with acceleration voltage at 20 kV, delay time 50-250 nsec, 300 laser shots per spectrum, laser intensity 1500-1700, with the digitizer vertical scale set at 500 mV. Spectra were calibrated externally or internally using the InvitroMass LMW calibrant kit (Invitrogen-Life Technologies, Carlsbad, Calif.). The PMF (peptide mass fingerprinting) of BSA (SwissProt accession number P02769) was analyzed by Voyager Explorer software (Applied Biosystems, Foster City, Calif., USA) as well as MASCOT® (Matrix Science, Boston, Mass., USA).

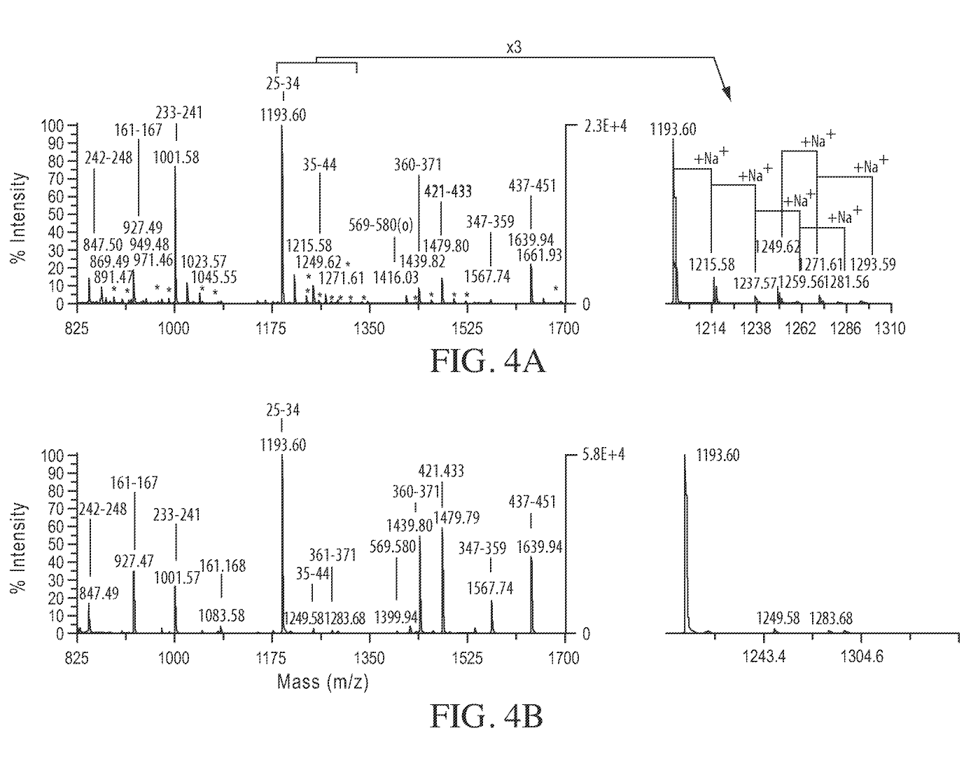

The results (FIG. 4) show the benefit of 1MB in MALDI-MS TOF analyses of trypic peptides. Spectrum A (no IMB) is more complex than spectrum B (with IMB). Many of the peaks unique to spectrum A comprise a cluster of repeating peaks that are 22 Da apart, suggesting that these peaks are sodium adduct clusters. The expanded view of the mass region between m/z 1190 and m/z 1310 from Spectrum A (no IMB) clearly illustrates two cluster series: one beginning with m/z 1193.6 and another series beginning with m/z 1249.62. In contrast, Sodium adduct clusters are clearly absent from Spectrum B (+IMB).

Moreover, two low abundance peaks (m/z 1083.6, 1283.7) are visible when FMB is present (Spectrum B). The m/z 1083.6 signal corresponds to the peptide 161-168, which overlaps with 161-167 (m/z 927.5) as it is the result of a single missed cleavage site. The m/z 1283.7 (361-371) also overlaps with the corresponding sequence of an observed peptide peak (m/z 1439.8, 360-371). Interestingly, m/z 1283.7 appears at lower abundance than m/z 1439.8 and, assuming that m/z 1283.7 and 1439.8 have equivalent ionization efficiencies (although m/z 1283.7 is likely to have a slightly higher ionization efficiency), this would suggest that trypsin cleavage occurs more frequently at R359 than R360. In any event, detection of these low abundance species only in spectrum B emphasizes the benefit of IMB in MALDI-MS TOF analyses.

These results include two interesting phenomena: (1) trypsinized BSA in 100 mM ABC-7.8 displayed a significant number of Na+ adducts (which we conclude is a contaminant originating from the purified BSA itself), and (2) BSA trypsinized in the presence of 250 mM NDSB-201 displayed no sodium adducts. Proteolysis of BSA performed in solution and analysis of the PMF yielded ˜46% sequence coverage in the absence of NDSB-201 and ˜64% in the presence of 250 mM NDSB-201 (average MASCOT® scores of 96 and 114, respectively). That is, like BLEND I, NDSB-201 increases the extent of sequence coverage.

Like BLEND I, NDSB-201 may be used as an additive during “in-gel” proteolysis. Experiments with “in-gel” tryptic digests of Cytochrome P450 were carried out as described above except that NDSB-201 was present in the digest solution. As with BLEND I, 250 mM NDSB-201 enhanced sequence coverage by MALDI-MS over the control (44% for NDSB-201 versus 40% for the control).

### Example 7

### NDSB-201: Bradykinin Ion Sequestration

The previous Example suggests that NDSB-201 prevents sodium from binding to BSA proteolysis products. An experiment was devised to investigate whether 250 mM NDSB-201 can titrate sodium adducts with increasing concentrations of NDSB-201 versus Na⁺. The peptide Bradykinin (monoisotopic M.W. 998.5786) was used as a test peptide for the titration studies.

Lys(-Des-Arg9, Leu8)-Bradykinin [H-Lys-Arg-Pro-Pro-Gly-Phe-Ser-Pro-Leu-OH (C47H75N13O11) (SEQ ID NO:9)] from Calibrant II of InvitroMass LMW calibrant kit (Invitrogen) was diluted to a final 1 mM concentration in either deionized water (negative control) or in 250 mM NaCl (Sigma-Aldrich, St. Louis, Mo.). The calibrant was then mixed 1:1 with various dilutions of NDSB-201 and spotted onto the MALDI target as described above. MALDI-TOF MS analysis was conducted in positive linear mode. Quantification of sodium sequestration by NDSB-201 was performed by analyzing the ion intensity of a centroid peak corresponding to the +Na⁺ adduct (m/z 1020.6) of the Bradykinin component (m/z 998.6). The average ion intensity of four spots was plotted against the respective concentration of 250 mM NDSB-201.

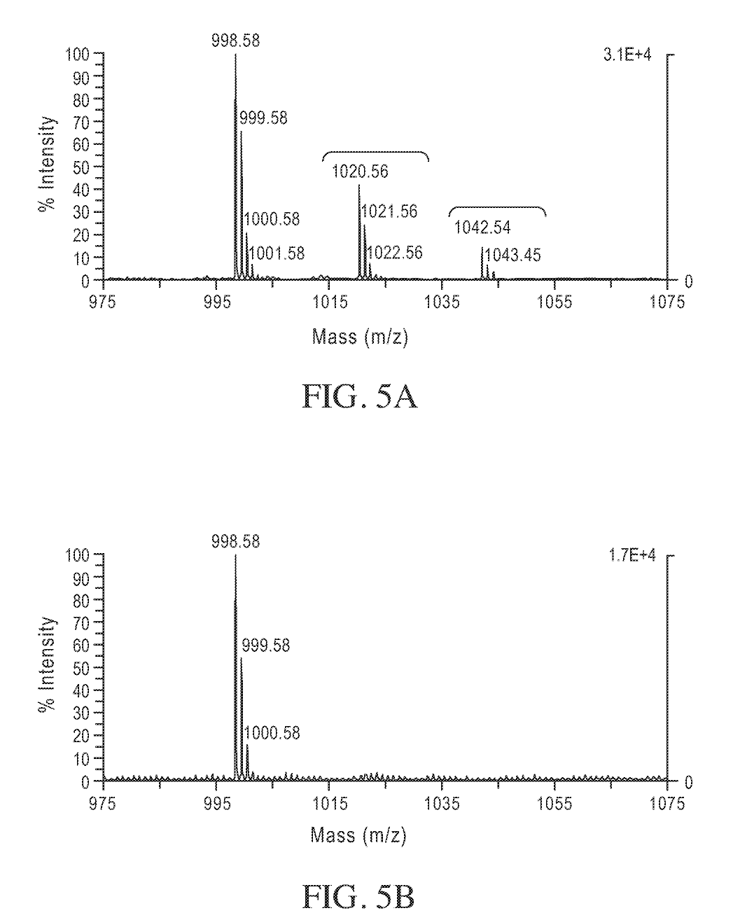

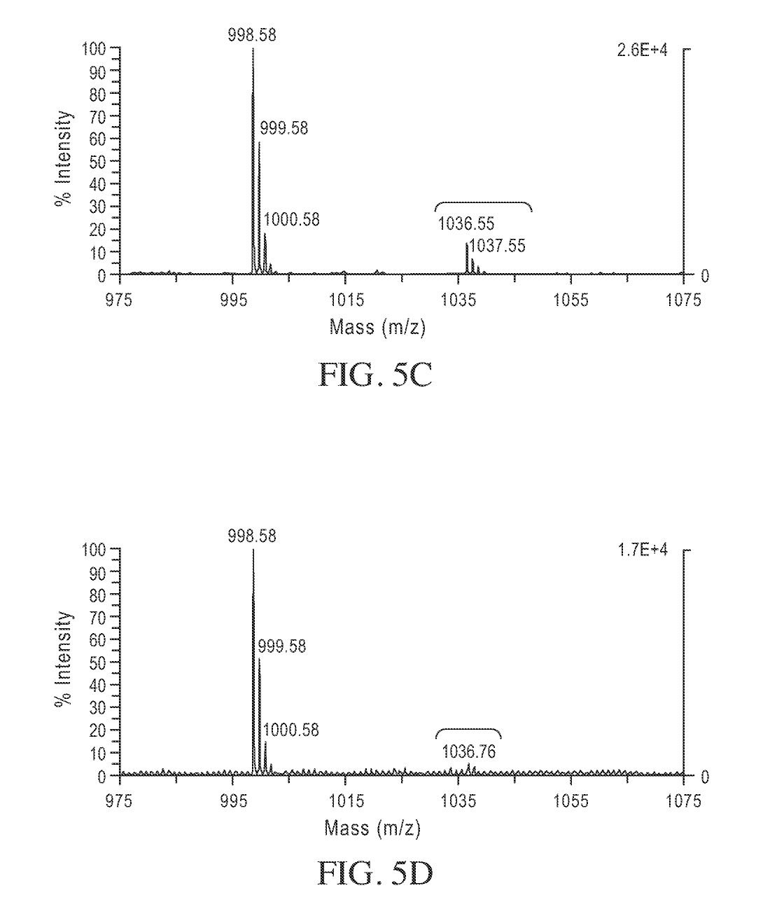

FIG. 5 shows that, under these conditions, 100 mM NDSB-201 is sufficient to eliminate the formation of sodium and potassium adducts. Bradykinin was analyzed in the presence of 50 mM NaCl (A and B) or 50 mM KCl (C and D). The sodium adducts in (A) (m/z 1020.56, 1042.54), and the potassium adduct in (C) and (D) (M/z 1036.55), are identified by green font. When Bradykinin in 50 mM NaCl (B) or 50 mM KCl (D) are co-spotted with 0.4× IMB 1:1 (v/v), the adducts are markedly reduced or eliminated [compare (A) to (B) and (C) to (D)].

The preceding experiment suggests that Bradykinin is a good test peptide to monitor adduction of sodium, and that NDSB-201 can eliminate, or at least substantially reduce, the formation of monovalent adducts. Thus, a titration experiment was carried out to determine if the removal of sodium adducts from Bradykinin occurs in an NDSB-201 concentration-dependent manner. Various concentrations of NDSB-201, ranging from 0-100 mM, were prepared using 1 mM of Bradykinin in 250 mM NaCl. The relatively high concentration of NaCl was selected to stringently test NDSB-201 ability to eliminate adduct formation.

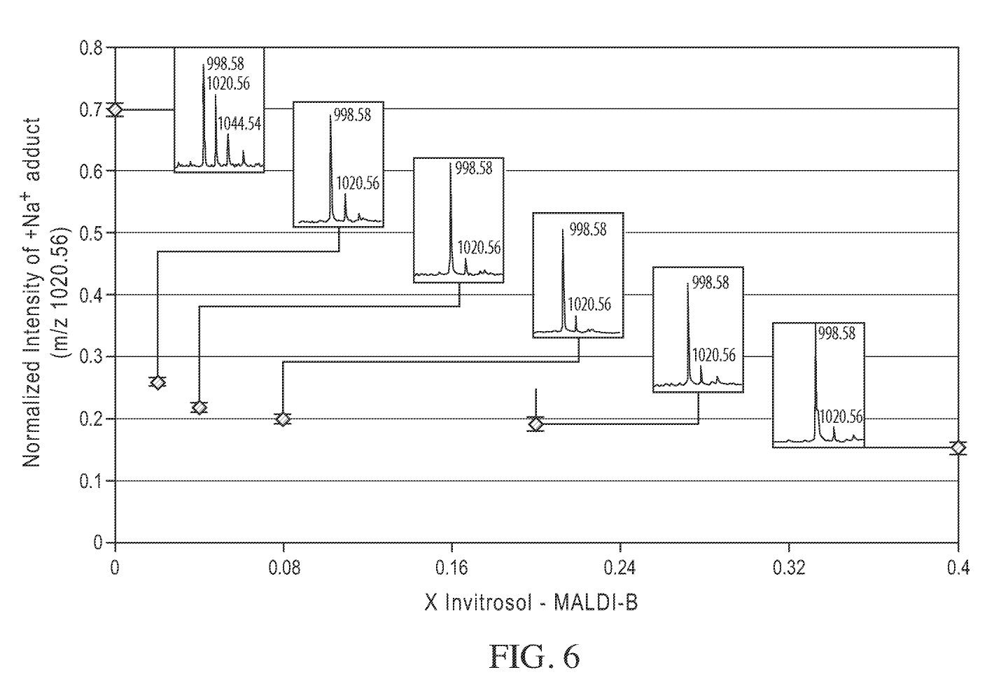

An analysis was performed of the normalized intensity of the sodium adduct m/z 1020.56 versus NDSB-201 concentration. The normalized intensity of the first sodium adduct peak (m/z 1020.56) was measured by the formula: (intensity of m/z 1020.56)/(intensity of m/z 998.58)+(intensity of m/z 1020.56), and plotted against the concentration of IMB (FIG. 6). The values on the X axis represent fractions of 1× concentration of IMB. A representative spectrum for each data point is shown in the plot. The ion intensity of m/z 998.58 in each spectrum is ˜1×10e4.

The results indicate that addition of 5 mM NDSB-201 is sufficient to reduce the intensity of adduct peaks, and that the relative signal for m/z 998.58 does not improve at concentrations of NDSB-201 higher than 20 mM. The relative intensity of m/z 1020.56 was reduced in an NDSB-201 concentration-dependent manner, suggesting that NDSB-201 competes for sodium binding sites on Bradykinin.

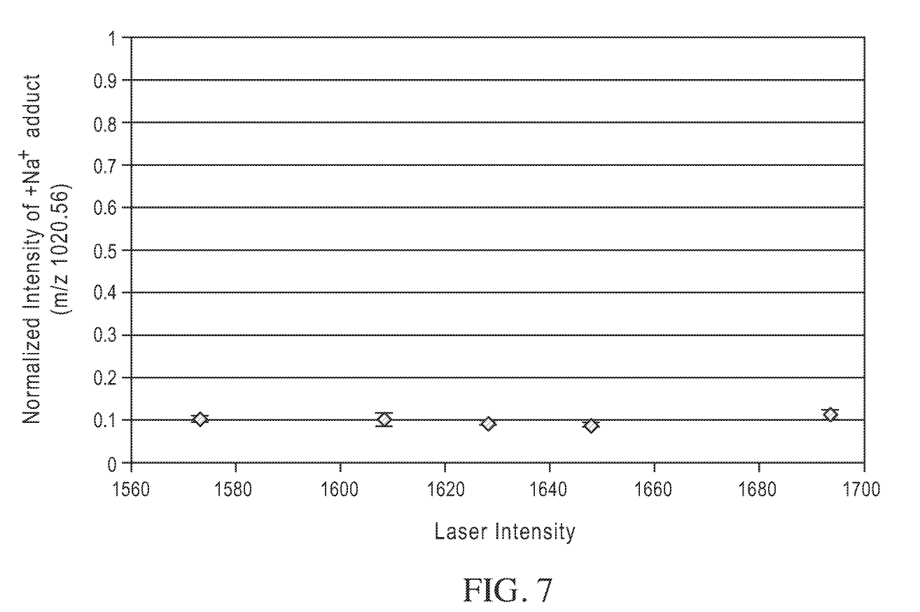

During the titration analysis we had observed that in order to maintain the ion intensity of the m/z 998.56 at approximately 1×10e4, the intensity of the laser setting had to be increased by 5-10% for the spots that contained greater than 0.1× IMB. It is possible that the relative intensities of unsodiated and sodiated Bradykinin might shift under this minor variation in laser fluence. An experiment was carried out to measure the normalized intensity of m/z 1020.56 (from Bradykinin in 125 mM NaCl) under a range of laser power settings. The results are shown in FIG. 7. The normalized intensity of the first sodium adduct peak (m/z 1020.56) was measured by the formula: (intensity of m/z 1020.56)/(intensity of m/z 998.58)+(intensity of m/z 1020.56), and plotted against the laser intensity (1500-1700). The values on the X axis represent laser intensity units. The results (FIG. 7) suggest that the relative intensities of unsodiated and sodiated Bradykinin are independent of the laser setting throughout the range tested.

### Example 8

### Testing Chaotropic Additives for Use with MS-Compatibile Solubilizers

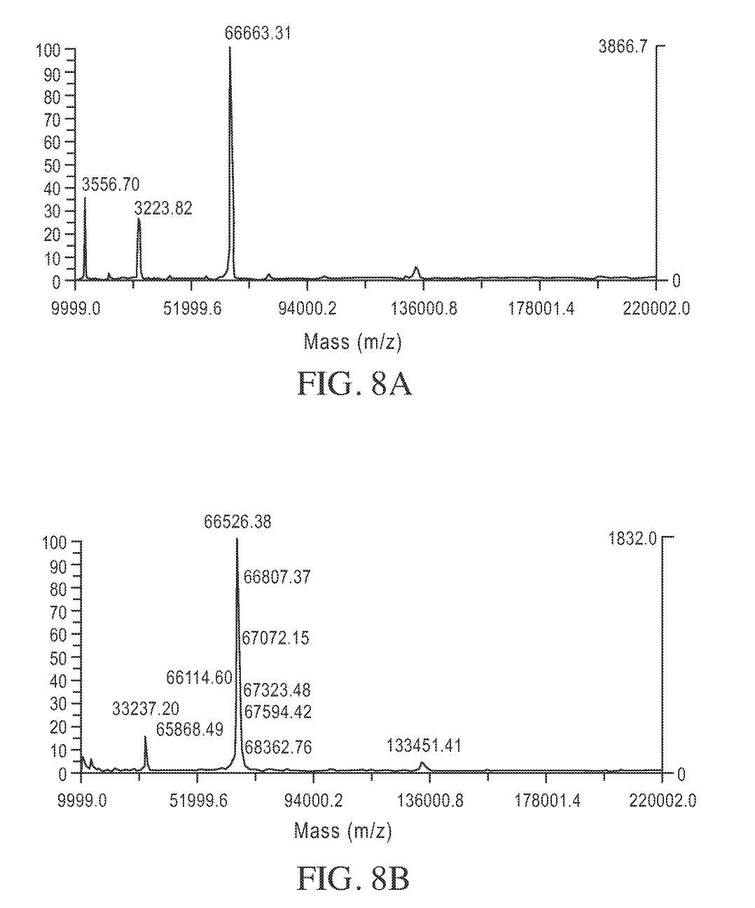

Although high concentrations of chaotropes such as urea and thiourea suppress ionization of proteins by MALDI, lower concentrations (˜0.7 M) are well tolerated. The MALDI compatibilities of 0.7 M urea and thiourea as additives to BLEND I and NDSB-201 were tested. FIG. 8 shows the MALDI-MS spectra of BSA in the presence and absence of BLEND I with 0.7 M urea and 0.7 M thiourea. There is little to no significant ionization suppression, although FIG. 8B indicates that urea and thiourea cause slight peak broadening. Suppression of ionization increases over time as the thiourea breakdown products begin to interact with the analyte protein, so it is preferable that the chaotropic mix be made fresh daily. Preferably, if urea and thiourea are to be used for analysis of peptides, the chaotrope concentration should not exceed about 0.35 M.

### Example 9

### Matrix Preparation with MS-Compatibile Solubilizers

During the course of these studies, it was observed that the manner in which solubilizer-MALDI samples are mixed with the MALDI matrix and applied to the MALDI target plate may require optimization. Without wishing to be bound by any theory in particular, the reasons for optimization may include the fact that surfactants are designed to break up intramolecular associations (thus preventing precipitation), whereas proper ionization by MALDI requires effective crystallization, a process that requires intramolecular contacts. Additionally, surfactants may lower the surface tension and inhibit droplet formation, and may thus dilute the sample across the surface area.

A “sandwich” spotting protocol that may circumvent or limit these problems is as follows. A matrix solution is prepared, either a saturated solution of sinapinic acid in 50% acetonitrile/0.1% TFA for intact proteins, or a saturated solution of alpha-cyano-4-hydroxycinnamic acid in 50% acetonitrile/0.1% TFA, mixed 1:1 with 50% acetonitrile/0.1% TFA for peptides. The matrix solution (typically, 0.5 μL) is spotted on the plate first. Once dry, the 0.5 μL of sample is spotted over it and allowed to dry. Then, another 0.5 μL of the matrix solution is spotted over that and dried.

### Example 10

### Development of Surfactant/Detergent Blends for LC and LC/MS (Blend II)

During the study of Invitrosol MALDI formulations, RP-HPLC was used to develop a method to compare the retention time of the different blend components and to verify any changes in stability. It was observed that several Invitrosol MALDI components have stable and predictable retention times and do not elute within the typical retention time range of proteins and peptides. The non-detergent sulfobetaine NDSB-201 was tested and found to elute in the void volume in a typical RP-HPLC run. However, NDSB-201 alone is not enough to solubilize extremely hydrophobic proteins such as Vitamin K carboxylase. Hence, blends of NDSB-201 and detergents were tested, including those of the sulfobetaine class (such as SB14 and SB10) as well as other non-detergent sulfobetaines such as NDSB-256. Each individual surfactant was tested by C18-RP HPLC for its binding and elution properties. Components were evaluated based on their ability to (1) elute in the void volume or (2) elute in a distinct peak at a solvent concentration that is high enough to ensure separation from eluting peptides, and (3) their ability to not elute in subsequent runs as “ghost peaks”.

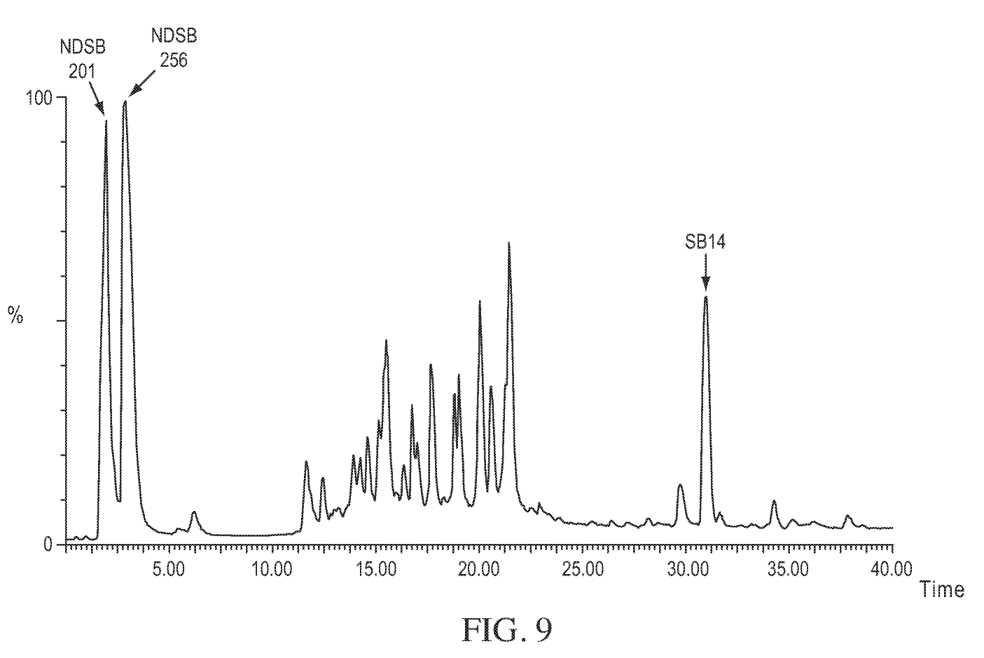

Although some were RP-HPLC compatible (NDSB 201 and SB 14), others were not (C8 phenyl). A MS-compatible surfactant blend was formulated that included NDSB 201, NDSB 256 and SB14, components that eluted in distinct peaks. FIG. 9 shows chromatographic separation of Cytochrome P450 digested in the presence of BLEND II where the surfactant components elute separately from peptides.

This blend, referred to herein as “BLEND II”, is preferably used for liquid chromatography (LC), including without limitation HPLC and RP-HPLC.

Blend II is also compatible with isoelectric focusing, including isoelectric separation methods that use immobilized pH gradients, such as immobilized pH gradient (IPG) strips, and pI-based separation methods such as capillary electrophoresis. Isoelectric focusing can also be performed using column chromatography.

TABLE 8
FORMULATION OF 1X BLEND II

| Component | Concentration |
|---|---|
| NDSB-201 | 50 mM |
| NDSB-256 | 50 mM |
| SB-14 | 0.01 mM |
| Ammonium Bicarbonate, pH 7.8 | 50 mM |

An alternate composition that is also compatible with HPLC, RP-HPLC, capillary electrophoresis, and immobilized pH gradient (IPG) separation is Invitrosol C:

TABLE 9
FORMULATION OF INVITROSOL C

| Component | Concentration (1x) |
|---|---|
| NDSB-201 | 25 mM |
| NDSB-256 | 25 mM |
| SB-14 | 0.22 mM |
| Ammonium Bicarbonate, pH 7.8 | 25 mM |

Note that deionized, sodium free water is preferably used in the production of these blends.

### Example 11

### Blend II: Effective Concentrations

Appropriate effective concentrations for BLEND II in different procedures and under different conditions were determined based on the following parameters and experiments.

### Preventing Precipitation of Hydrophobic Proteins During Dialysis

Buffer exchanges using a Centricon device (Millipore) on Cytochrome P450 3A4 and 2D6 (Invitrogen/PanVera) were carried out in order to exchange the 20% glycerol included in the stock storage buffer for BLEND II. Cytochrome P450 was selected as a test subject because it contains a transmembrane segment within the first 30 N-terminal residues and thus requires a surfactant to be soluble in an aqueous solution. Buffer exchange was also carried out on affinity purified nicotinic acetylcholine receptor (nAChR) from Torpedo Pacifica electroplax organ reconstituted in mixed phospholipids vesicles in Tris buffer.

### Resolubilizing Acetone-Precipitated Proteins During Dialysis

Buffer exchanges, and detergent removal, can be performed by selectively precipitating a protein in an incompatible solution using acetone. Typically, it is difficult to resolubilize pelleted hydrophobic proteins without the aid of surfactants. The ability of BLEND II to resolubilize of acetone-precipitated proteins was tested as follows.

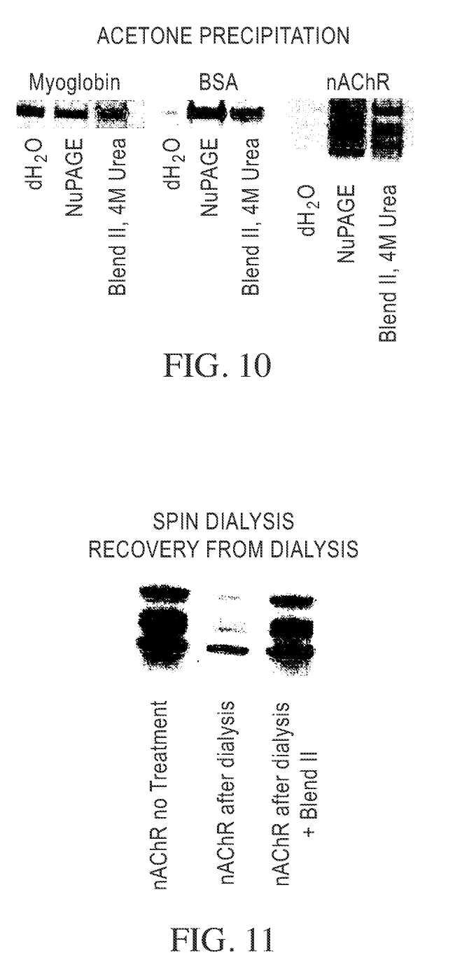

nAChR was used as model test hydrophobic proteins. Acetone precipitated proteins (prepared essentially according to the Acetone Precipitation Protocol in Example 14, below) were resolubilized in various concentrations of BLEND II (FIG. 10). Similarly, proteins were dialyzed against various concentrations of BLEND II (FIG. 11). Myoglobin and BSA were used in parallel as soluble protein controls to assess losses inherent to each exchange protocol. Equivalent samples of protein before and after exchange by dialysis or precipitation were analyzed by SDS-PAGE analysis to determine if significant losses in protein recovery occurred. The SDS-PAGE for acetone precipitated proteins is shown in FIG. 10. Cytochrome P450 and myoglobin were recoconstituted in water, NuPAGE® LDS Sample Buffer (Invitrogen) or 1× BLEND II. The results show that BLEND II is nearly as effective as the NuPAGE® buffer in resolubilizing the Cytochrome P450 pellet.

### Enhancing Amino Acid Sequence Coverage by at Least About 20%

Samples of Cytochrome P450 and nAChR were digested with trypsin in solution in the presence of 1× BLEND II. Samples were analyzed by LC-MS analysis. A comparative analysis between samples digested in the presence or absence of Invitrosol was not possible because, in the absence of Invitrosol these samples are not soluble, or these samples require a MS incompatible surfactant. Thus, results obtained from in-solution digests with BLEND II were compared to those from in-gel digests in the absence of any added surfactant. The results are shown in Table 10.

TABLE 10
PMF RESULTS

| | In-solution tryptic | | | |
|---|---|---|---|---|
| | digest with Chroma- | | In-gel tryptic digest | |
| | Sol detergent | | with BLEND II | |
| | Confidence | Sequence | Confidence | Sequence |
| Subunit | score | Coverage | score | Coverage |
| (P02710) | 670 | 32% | 115 | 16% |
| Alpha chain | | | | |
| precursor | | | | |
| (P02712) | 470 | 13% | 68 | 10% |
| Beta chain | | | | |
| precursor | | | | |
| (P02714) | 641 | 23% | 54 | 15% |
| Gamma chain | | | | |
| precursor | | | | |
| (P02718) | 475 | 13% | 134 | 10% |
| Delta chain | | | | |
| precursor | | | | |

The average enhanced Mascot sequence database score for the four nAChR subunits was 7 fold higher using Invitrosol, and the average enhanced sequence coverage was 1.5 fold higher.

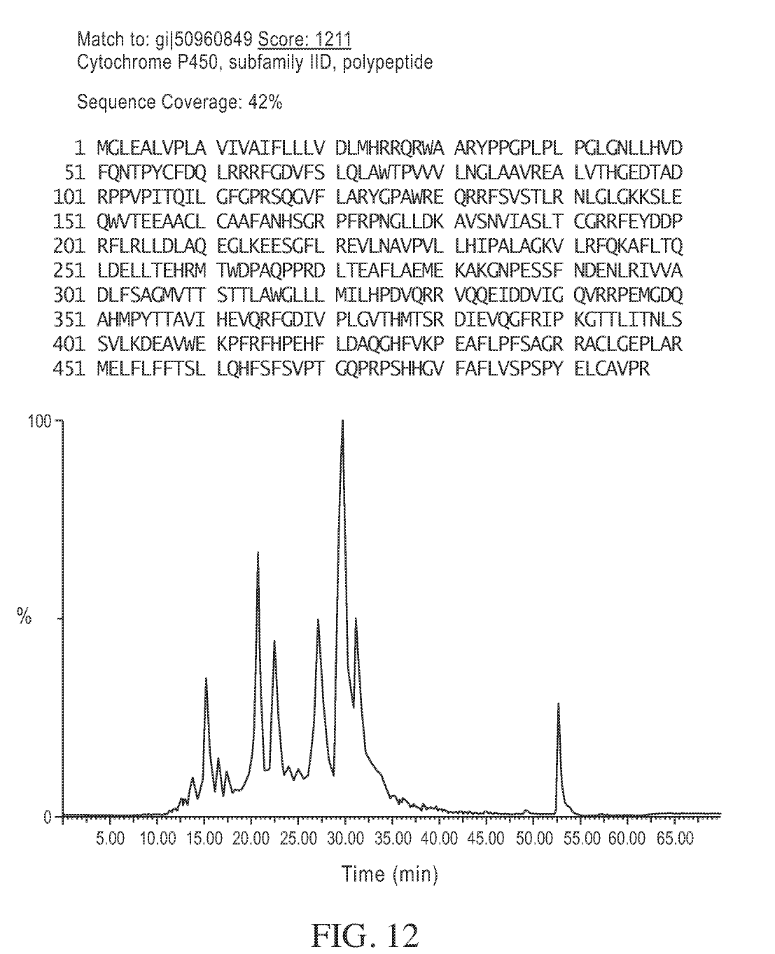

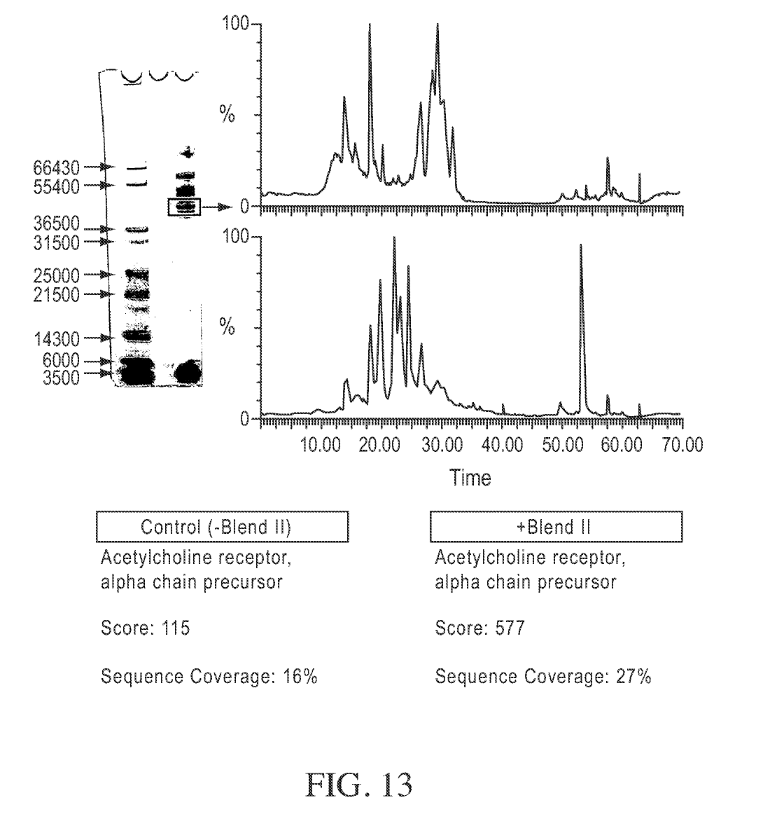

In solution digests of Cytochrome P450 followed by LC-MS are not technically feasible due to the abundant presence of glycerol and salts, which interfere with RP separations. These problems, however, can be circumvented using Invitrosol-LC, which can be used to resuspend acetone precipitated protein or substitute glycerol using dialysis. FIG. 12 shows a typical LC-MS total ion chromatogram for a sample of P450 in 1× BLEND II digested with trypsin. Using the combination of trypsin digestion in BLEND II and LC-MS, statistical scores from Mascot sequence database searches were quite high (1211 score in FIG. 12).

### “In-Gel” Digestion

In order to identify a protein from a peptide map fingerprint (PMF), several parameters must be optimized. One of these parameters is sequence coverage. While it is possible to identify a protein with low sequence coverage based on the PMF alone, one may not be able to distinguish between related gene products or splice variants. Low sequence coverage is often the result of low solubility of peptides covering the hydrophobic regions of a protein. Even a soluble globular protein contains hydrophobic domains in its core. During a typical “in-gel” proteolysis protocol, the enzymatic digest is performed in an aqueous solution where hydrophobic fragments may irreversibly precipitate. Therefore, we have tested the application of BLEND II during “in-gel” proteolysis.

A sample containing 75 pmol of Cytochrome P450 1A2 was prepared and separated by SDS-PAGE using traditional protocols. A band at ˜60 kDa corresponding to P450 was excised and de-stained with 50% acetonitrile/25 mM ammonium bicarbonate pH 8.0. 200 ml of 100% acetonitrile was added to the gel piece and then dried down using a speed-vac apparatus. The sample was then rehydrated in a 10 ng/ml solution of trypsin (Promega) in 25 mM ammonium bicarbonate pH 8.0 plus 1× BLEND II and incubated overnight at 37∞C. After proteolysis, the digested peptides were extracted using one wash of 10 ml 5% FA, and an additional 10 ml 25% acetonitrile/5% FA wash. The extracted samples were analyzed by RP-HPLC and the resulting mass/ions identified by the Mascot sequence database search program (Matrix Science). Comparing the digested sample with and without BLEND II & LC/MS, we saw modest improvements in the sequence coverage (40% without and 48% with BLEND II).

Whereas modest improvements in sequence coverage were observed for in-gel digestion of Cytochrome P450 in the presence of BLEND II, dramatic improvements were observed with nicotinic acetylcholine receptor. Affinity purified Torpedo Pacifica nAChR was separated by SDS-PAGE followed by in-gel trypsin proteolysis in the presence of BLEND II. FIG. 5 illustrates the dramatic improvements in Mascot score and sequence coverage when using BLEND II. Specifically, the sequence coverage improved from 16% to 27% and the Mascot score improved from 115 to 577.

### Example 12

### Blend II: Stability Studies

Stability testing for the original BLEND II was performed over a period of 1 week at 37° C. An HPLC-based method, whereby samples are separated by C18 reverse phase chromatography while monitoring the eluted components by absorbance at 210 nm, was used. Chromatograms of the freshly prepared control sample and the test samples were compared and analyzed for the presence of additional peaks or shifts in the retention times. These experiments showed that the components of BLEND II had not decayed by the end of the test period. HPLC analysis of BLEND II that had been freshly prepared, stored for 45 days at RT or for 1 day at 60° C. showed no differences between the samples.

In the absence of chaotropes, surfactant blends will be stable at room temperature for at least two weeks and three months at 2° C. to 8° C. As initially provided, surfactant blends may be aliquoted and stored at −20° C. for eighteen months. Upon thawing, the blends may be diluted to reach the 1× concentration recommended for most purposes.

### Example 13

### Blend II: Representative Kit

A representative BLEND II kit comprises a surfactant blend and a standard, separately contained and/or packaged, co-packaged in a box or other container.

BLEND II is provided at 5x concentration in a clear polypropylene screw cap bottle. Sufficient BLEND II will be provided to perform ˜75 detergent/buffer exchange procedures (5 ml).

A representative standard is a Tryptic Digest of BSA standard prepared in BLEND II. A representative amount of standard is 25 ml of 1 μg/μL BSA tryptic digest in BLEND II. The BLEND II solution and the standard are provided separately, each in a 1.7 ml polypropylene screw cap microcentrifuge vial with a removable cap (from, e.g., VWR).

The kit may be warehoused at −20° C. and shipped at either −20° C. or 4° C. The customer will be advised to store the product at 4° C. if it is to be used in under 2 months, or aliquoted at −20° C. until reaching the expiration date (18 months post production).

The kit optionally includes a product manual which will cover storage conditions, protocols for several applications, and a link to an Invitrogen website that can keep the customer informed of new blends as well as new application protocols.

### Example 14

### Blend II: Protocols

### Acetone Precipitation Protocol

BLEND II surfactant blends are formulated to be directly compatible with LC & LC/MS analysis at 1× concentration. The following protocol is designed for intact protein samples that contain solubilizers such as CHAPS, PEG, Glycerol, SDS, and salt concentration which may interfere with trypsin activity.

1. Add 80% (v/v) of cold acetone to the mixture and incubate on dry ice for at least 3 hrs.

2. Centrifuge the tube at 14,000× rpm at 4° C. for 10 minutes.

3. Carefully remove the supernatant.

4. Wash the pellet with cold acetone twice.

5. Air dry the pellet.

6. Add enough 1× LC & LC/MS compatible detergent to the pellet to rich the suitable concentration (depends on the experiment and the instrument that the sample is being analyzed).

7. Vortex for about 1-2 minutes.

8. Incubate at 60° C. for 5 minutes.

9. Vortex for about 1-2 minutes.

10. Incubate at 60° C. for another 10 minutes or until the pellet is completely dissolved.

### Buffer Exchange Protocol

The following protocol is designed to remove buffer solution components that may interfere with LC and LC-MS analysis.

1. Mix your intact protein sample with 5× BLEND II, and incubate at 37° C. for 10 min. The total volume of the sample to be dialyzed should not exceed 100 ml.

2. Prepare and wash the dialysis device adding 100 μL of ultrapure diH₂O and centrifuging the device for 5 minutes at 2,500×.

3. Using a pipet transfer the mixture of the sample LC & LC/MS compatible detergent without touching the membrane.

4. Centrifuge for 20 minutes at 12,000 rpm. (Note: it is safe to check the volume of the sample in the centricon device every 5 minutes to avoid the driness and eventually sample lost)

5. Add another 100 μL of 1× LC & LC/MS compatible detergent and centrifuge for another 20 minutes.

Repeat step 5 for 2-3 times.

### Trypsin Digestion Protocol

The following protocol is intended to digest an intact protein with sequencing grade trypsin. This protocol is intended for proteins that have already undergone buffer exchange as described above.

1. Reconstitute lyophilized Trypsin using 25 mM Ammonium Bicarbonate at pH 8.0. Ideally, the final solution should have an enzyme to substrate ratio of 1:25-1:100 (w/w), and a final concentration of 25 mM ammonium bicarbonate.

2. Incubate the mixture for 12-18 hrs at 37∞C.

3. The sample may be analyzed directly by RP-HPLC using the following steps:

(a) Depending on the column size (for a 4.6 mm ID column load 10-100 pmol of digested protein, for a 100-300 μm ID column load 0.5-2 pmol) inject the appropriate bolus of sample.

(b) Monitor the wavelength at 214 nm and, if using multivariable wavelength detector, at 280 nm.

(c) Run the appropriate gradient protocol (this protocol must be determined empirically for each protein preparation, but a generic gradient should include a ramp from 5%-70% Acetonitrile in water with 0.1% TFA over 50 minutes) with an initial offline (if analyzing by ESI-MS) wash period of no less than 10 min in order to allow BLEND II components that elute in the void volume to pass through undetected.

### In-Gel Trypsin Digestion Protocol

Cut the appropriate gel-band using the tip of a P-1000 pipettor,

mince the gel into small pieces.

Wash the band in 50% acetronitrile, 25 mM AMBC, pH 8.0

Repeat until the band is sufficiently destained

Add 200 uL of ACN to dehydrate, incubate for 5-10 min at RT

Speed-vac

Add 5-10 uL of a 10 ng/μL solution of trypsin (in 100 mM ABC pH8) in 1× Invitrosol LC. Incubate ov/nt at 37 degrees

Add 10-15 μL of 2.5% TFA, incubate for 30 min at RT

Collect sup

Add 10-15 μL of 2.5% TFA/50% ACN, incubate for 30 min at RT

Pool sup's for a combined 25% ACN/2.5% TFA solution

LC-MS analysis (if ESI-MS is to be used substitute TFA with an equivalent concentration of formic acid).

### Example 15

### Illustrative Detergents, Non-Detergents, and Other Compositions for MS-Compatible Solubilizers

### Alkyl Glycosides

One type of MS-compatible solubilizer is an alkyl glycoside having the structure

R—Z—(CH₂)ₓ—CH₃ [I]

wherein:

Z can be O, S, Cl, I, Fl, Se, Br;

x=1-20;

when R=glucose, x=1-8; and

when R=maltose, x=1-11.

Representative compounds wherein Z is O and R is maltose include without limitation n-ethyl-beta-D-maltoside (x=1), n-propyl-beta-D-maltoside (x=2), n-tetryl-beta-D-maltoside (x=3), n-pentyl-beta-D-maltoside (x=4), n-hexyl-beta-D-maltoside (x=5), n-heptyl-beta-D-maltoside (x=6), n-octyl-beta-D-maltoside (x=7), n-nonyl-beta-D-maltoside (x=8), n-decyl-beta-D-maltoside (x=9), n-monodecyl-beta-D-maltoside (x=10), and n-dodecyl-beta-D-maltoside (x=11).

Representative alkyl glycosides having structures wherein Z is O and R is glucose include without limitation n-ethyl-beta-D-glucopyranoside (x=1), n-propyl-beta-D-glucopyranoside (x=2), n-tetryl-beta-D-glucopyranoside (x=3), n-pentyl-beta-D-glucopyranoside (x=4), n-hexyl-beta-D-glucopyranoside (x=5), n-heptyl-beta-D-glucopyranoside (x=6), n-octyl-beta-D-glucopyranoside (x=7), and n-nonyl-beta-D-glucopyranoside (x=8).

### Sulfobetaines

One type of MS-compatible solubilizer is a sulfobetaine having the structure

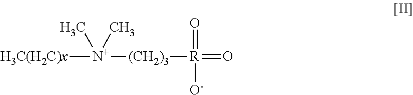

wherein:

R can be S, P or C; and

x=1-20.

### Non-Detergent Sulfobetaines (NDSBs)

Another type of MS-compatible solubilizer is a non-detergent sulfobetaine having any of the following structures III-VI.

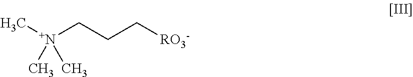

wherein R is S, P or C.

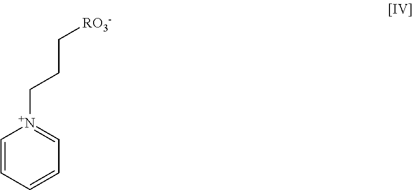

wherein R is S, P or C.

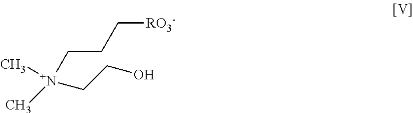

wherein R is S, P or C.

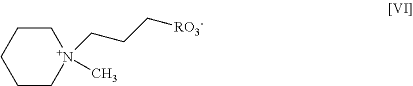

wherein R is S, P or C.

### Bile Acids

Another type of MS-compatible solubilizer is a bile acid having the structure

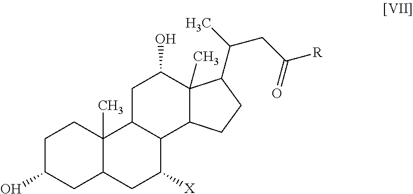

wherein:

R is a non-detergent sulfobetaine; and

X can be H or OH.

### Rabilloud Detergent Variants

Another type of MS-compatible solubilizer is a Rabilloud detergent variant having the structure

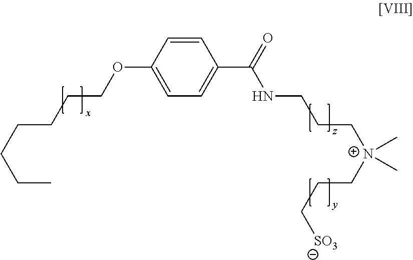

wherein x, y and z are independently selected selected from the group consisting of

x=0-25, preferably 0-10;

y=0-15, preferably 0-10; and

z=0-15, preferably 0-10.

In some instances, z=0, 1, 2, 3, 4 or 5; y=0, 1, 2, 3, 4 or 5; and/or x=0, 1, 2, 3, 4, 5, 6, 7, 8, 9 or 10.

### Example 16

### MALDI-MS Solubilizer Kit

A representative kit comprising one or more of the MALDI-compatible solubilizers of the invention is packaged in a box and includes the following elements in a total of 2 solubilizing solutions in screw top bottles, 5 compositions in screw cap vials and, optionally, empty vials for sample manipulation. Deionized, sodium free water is used in the production of all solutions.

A. One (1) clear polypropylene screw cap bottle containing 10 ml of 5× BLEND I (formulation in Table 5) (sufficient detergent blend to perform ˜75 detergent/buffer exchange procedures);

B. One (1) clear polypropylene screw cap bottle containing 25 ml of 2× BLEND II (500 mM NDSB-201) (sufficient detergent blend to perform ˜75 detergent/buffer exchange procedures);

C. One (1) 1.7 ml polypropylene screw cap microcentrifuge vial (VWR International, West Chester, Pa.) containing 25 μL of 90 kDa InvitroMass standard (Invitrogen) (sufficient for at least 10 experiments);

D. One (1) vial containing 20 mg of sinapinic acid;

E. One (1) vial containing 20 mg of alpha-cyano-4-hydroxycinnamic acid;

F. One (1) 1.7 ml polypropylene screw cap microcentrifuge vial with red cap (VWR) containing 1 ml of 0.1% trifluoroacetic acid (solvent for mixing MALDI matrices);

G. One (1) 1.7 ml polypropylene screw cap microcentrifuge vial with red cap (VWR) containing 1 ml of 100% acetonitrile (1 ml) (solvent for mixing MALDI matrices);

H. Optionally, four (4) to 20 empty 1.5 ml clear polypropylene microcentrifuge tubes;

I. Optionally, vials containing powdered chaotropes in optimal amounts or in mixed optimized proportion; and

J. A product manual that will describe storage conditions and experimental protocols. The product may be warehoused at −20° C. and shipped at either −20° C. or 4° C. The customer will be advised to store the product at 4° C. if it is to be used in under 2 months, or aliquoted at −20° C. until reaching the expiration date (12 months post-production):

Instructions for resuspension of MALDI matrices will also be included and are as follows: Mix 0.5 ml of acetonitrile solution to 0.5 ml of 0.1% TFA. Add 0.5 ml of this new solution to the “Sinapinic acid” vial and 0.5 ml to the “alpha-cyano-4-hydroxycinnamic acid” vial. These matrices are stable at 4° C. for about 2 weeks. In general, sinapinic acid is recommended for preparations of intact hydrophobic proteins and alpha-cyano-4-hydroxycinnamic acid is recommended for protein digests.

Sufficient documentation will be included to describe the application of this kit's components for (a) direct analysis and calibration of intact hydrophobic proteins; (b) exchange of protein material from MS-incompatible surfactants and/or buffers (the directions will describe general recommendations for the following specific contaminants: SDS, Triton X, CHAPS, and HEPES; and (c) performance and analysis of tryptic digests supported by the kit.

### Example 17

### Experimental Protocols

The following experimental protocols may optionally be included with a kit or other compositon of matter comprising a MS-compatible solubilizer of the invention.

### Direct Analysis and Calibration of Intact Hydrophobic Proteins

Invitrosol-MALDI surfactant blends are formulated to be directly compatible with MALDI-MS analysis at 1X concentration. The following protocol is designed for intact protein samples that do not contain any interfering contaminants such as CHAPS, PEG or SDS. Please refer to the “Buffer Exchange Protocol” below if your sample contains any of these materials.

1. Mix your intact protein sample with an MS-compatible solubilizer, 5× BLEND I or 2× (500 mM) NDSB-201, to obtain a final 1× BLEND I or 250 mM NDSB-201, and incubate at RT for 10 min. (NOTE: It is important that your final concentration of BLEND I or NDSB-201 not exceed 1×). Addition of chaotropes (urea and/or thiourea) up to, but not exceeding 0.35 M may be used for more hydrophobic proteins. Final content of analyte intact protein should be 50 fmol-10 pmol. (NOTE: Urea and/or thiourea (especially) should be prepared fresh from powder. Over the course of a single day, the decomposition of thiourea creates contaminants that hamper the MALDI ionization process significantly.)

2. Spot 0.5 μL of solution from the “Sinapinic Acid” vial onto the MALDI sample target. Allow the spot to dry at RT.

3. Spot 0.5 μL of sample solution mixed with MS-compatible solubilizer(s) at 1× final concentration. Allow the spot to dry at RT.

4. Spot another 0.5 μL of solution from the “Sinapinic Acid” vial onto the MALDI sample target. Allow the spot to dry at RT.

5. Onto another well on the MALDI target, or near the preceding sample, spot 0.5 μL of a 1:1 mixture of 90 kDa InvitroMass molecular mass standard with Sinapinic acid. Alternatively, the molecular mass standard can be co-spotted with the analyte. (NOTE: It may be necessary to empirically adjust the concentration of the InvitroMass standard relative to your protein to optimize the performance and preparation using internal standard.)

6. In linear mode, ramp the laser intensity up slowly until the desired signal-to-noise intensity is achieved. (NOTE: Due to the presence of MS-compatible solubilizer, a slightly higher laser setting may be required).

7. In order to calibrate, use average mass of 89,610 Da for the molecular mass standard.

### Buffer Exchange Protocol

Invitrosol-MALDI blends can be exchanged for commonly used surfactants that are incompatible with MS. The following exemplary protocol is designed to remove buffer solution components that may interfere with MALDI-MS analysis.

1. Mix your intact protein sample with an MS-compatible solubilizer, 5× BLEND I or 2× (500 mM) NDSB-201, to obtain a final 1× BLEND I or 250 mM NDSB-201, and incubate at RT for 10 min. (NOTE: It is important that your final concentration of BLEND I or NDSB-201 not exceed 1×). Addition of chaotropes (urea and/or thiourea) up to, but not exceeding 0.35 M may be used for more hydrophobic proteins. Final concentration of analyte intact protein should be no less 500 fmol.

2. The total volume of the sample to be dialyzed should not exceed 40 ml.

3. Prepare a dialysis solution by diluting either 5× BLEND I or 2× NDSB-201 (selected in Step 1) to 0.05×. Chaotropes need not be added to the dialysis solution.

4. Place ˜3 ml of dialysis solution in an inverted 15 ml conical tube cap.

5. Using forceps, place a single 25 mm filter-membrane (Millipore #VSWP02500) on top of the filled cap.

6. Pipet the sample solution onto the center of the filter membrane.

7. Allow exchange to take place for ˜1 hr at RT.

8. After 1 hr, fill another cap with dialysis solution. Place the cap alongside the cap with the drop and membrane. Using forceps, slowly drag the filter over the new cap. Allow exchange to proceed for another hour at RT.

9. If significant swelling of the drop occurs, a concentration step by speed-vac may be necessary. (NOTE: Do not speed vac to dryness. Do not concentrate beyond the original droplet volume.)

The sample may be analyzed directly by MALDI-MS using the protocol described above in steps 2 through 7 for “Direct Analysis and Calibration of Intact Hydrophobic Proteins”.

### Trypsin Digestion Protocol

The following protocol is intended to digest an intact protein with sequencing grade trypsin. This protocol is intended for proteins that have already undergone buffer exchange as described above.

1. Mix your intact protein sample from the “Buffer Exchange Protocol” (above) with ammonium bicarbonate pH 8 and trypsin solution. Ideally the final solution should have an enzyme to substrate ratio of 1:25-1:250, and a final concentration of 50 mM ammonium bicarbonate.

2. Incubate the mixture for 12-24 hrs at 37° C. (NOTE: Porcine trypsin is enzymatically active in the presence of up to 40% acetonitrile. Many researchers have observed accelerated trypsin digestion in the presence of 10-20% acetonitrile.)

3. The sample may be analyzed directly by MALDI-MS using the following steps:

4. Spot 0.5 μL of solution from the “alpha-cyano-4-hydroxycinnamic acid” vial onto the MALDI sample target. Allow the spot to dry at RT.

5. Mix sample in 1× BLEND I or 250 mM NDSB-201 and matrix solutions 1:1 (v/v) and spot 0.5 μL. Allow the spot to dry at RT.

6. Spot another 0.5 μL of solution from the “alpha-cyano-4-hydroxycinnamic acid” vial onto the MALDI sample target. Allow the spot to dry at RT.

Ramp the laser intensity up slowly until the desired signal-to-noise intensity is achieved. (NOTE: Due to the presence of Invitrosol-MALDI, a slightly higher laser setting may be required).

### Example 18

### Maxion AC: Effect of Silica Additive on Signal-to-Noise Ratio in MALDI-TOF MS Using Purified Proteins

The MALDI-MS analysis of a mixture of highly purified standards using the conventional mixture of alpha C (4-hydroxy-alpha-cyanno-cinnamic acid, CHCA) dissolved in 50% Acetonitrile, 0.05% TFA was compared to that of a mixture of 1:1 (w/w) of CHCA:silica. The latter was prepared as follows. A matrix solution (2 mg/ml) was prepared by dissolving 2.2 mg of solid alpha C in 1 ml of a matrix diluent that is 80% (v/v) HPLC grade acetonitrile and 0.05% (v/v) HPLC grade TFA (final volume, 1.1 ml). A resin solution (20 mg/ml) was prepared by dissolving 20 mg of Lichrosorb Si60 (5 μm) beads (EMD) in 1 ml of a solution of resin diluent [99% (v /v) HPLC grade acetonitrile and 0.01% (v/v) ACS grade ammonium hydroxide]. Immediately before use, the matrix and resin solutions were mixed 9:1 (v:v) to generate a matrix/resin solution. Typically, two (2) μL of conventional matrix solution or matrix/resin solution were combined with 1 μL of sample before spotting.

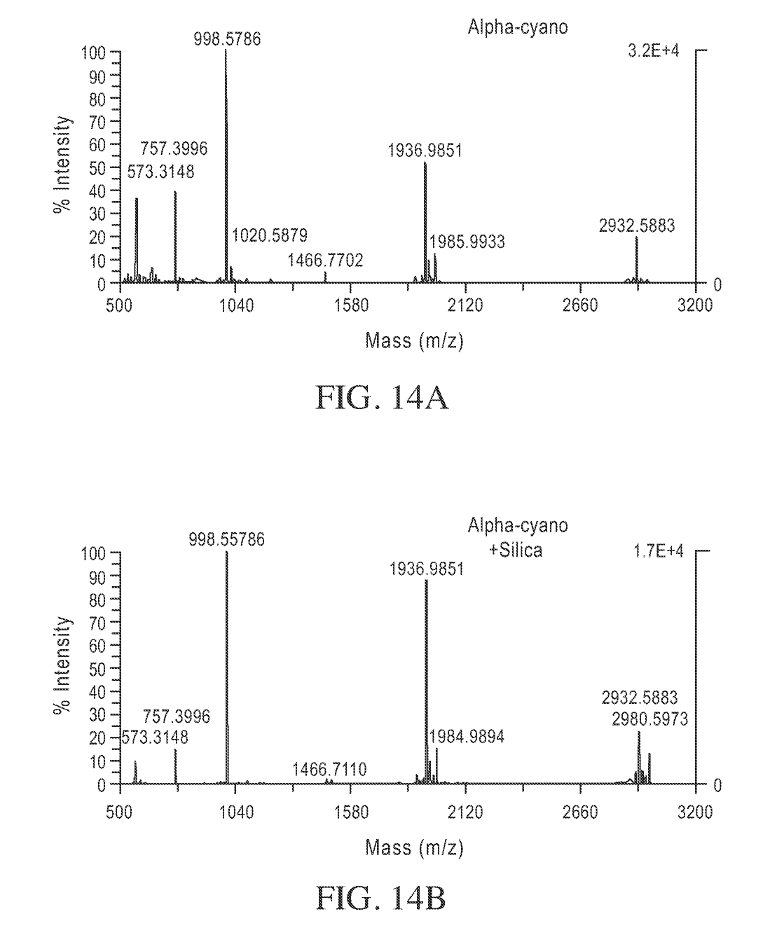

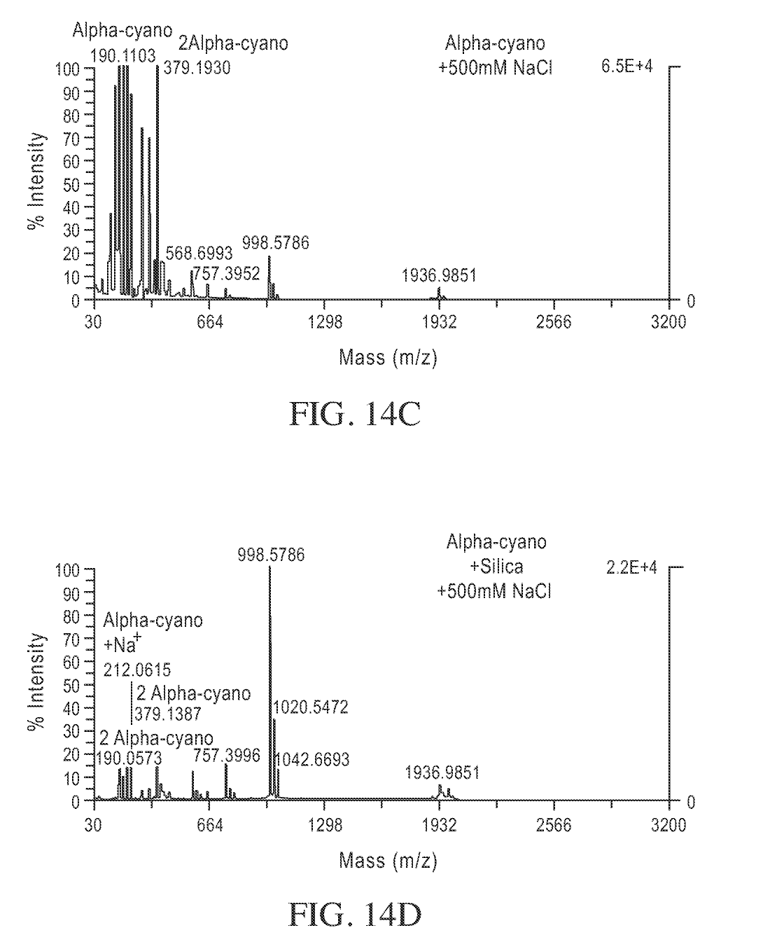

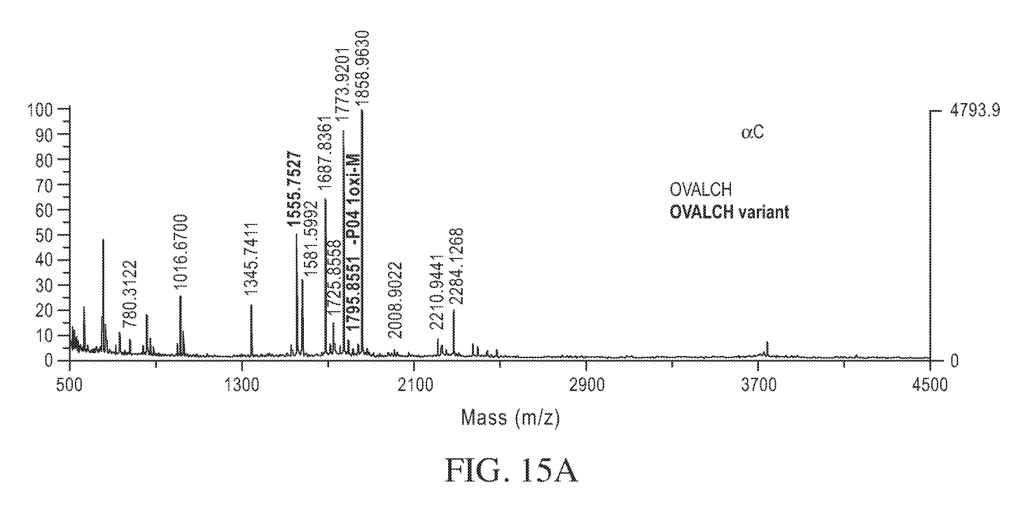

FIG. 14 shows the comparative analysis by MALDI-MS. The top two spectra show MALDI-MS analyses of a calibrant mixture [Bradykinin fragments (1-5, m/z 573.67; 1-7, m/z 757.87), Bradykinin Lys (-Des-Arg9, Leu8) (m/z 998.58), ACTH (1-16, m/z 1936.99), ACTH (1-24, m/z 2932.59)] in conventional alpha-cyanno (top left spectrum) and the alpha-cyano:silica mixture (top right spectrum). The total ion counts on the y axes indicate the improved signal to noise obtained with silica resin. There are significant gains in signal-to-noise across the spectrum despite the total ion counts decreasing from 2.3e4 to 1.7e4 at the same laser intensity.

The experiment was repeated in the presence of 500 mM NaCl in conventional alpha-cyano c (bottom left spectrum) versus silica resin (bottom right spectrum), where the low mass gate was opened to allow low-mass ions. The improved signal to noise ratio caused by the use of silica as a matrix additive is apparent. Without wishing to be bound by any particular theory, the improved signal-to-noise appears to result from higher crystal quality (smaller and uniform crystals) and resulting analyte desolvation. Moreover, the total ion counts drop as a result of the decrease in laser fluence (laser energy per unit of area) due to light scatter induced by the silica particles.

Another discernable difference is the improved signal-to-noise of +48 Da species associated with m/z 1936.99 and m/z 2932.57. This may be a result OF silica's ability to improve the ionization of analyte species with multiple oxidations (3×16 Da). These oxidized species are clearly visible with alpha-cyanno, but the intensity is markedly improved in the presence of silica. Without wishing to be bound by any particular theory, it is possible that these oxidations induce the ACTH peptides into a secondary structure that may cause precipitation and/or reduced association with the matrix.

### Example 19

### MaxIon AC: Effect of Silica Additive on Tryptic Digests of Purified Proteins

An experiment was performed where a number of proteins were independently proteolyzed with trypsin and analyzed by MALDI-MS. The object of the experiment was to compare the performance of MaxIon AC silica resin against conventional CHCA using a number of different proteins. Ovalbumin, fetuin, myoglobin, and beta-galactosidase were digested at high enzyme to substrate ratios (1:20) in an attempt to maximize the extent of proteolysis and the sequence coverage. The digests were analyzed by MALDI-MS in both CHCA and MaxIon AC silica resin. The spectra were then processed and the peaks analyzed against a sequence database by MASCOT Distiller. Table 10 lists the results of the MASCOT sequence identification confidence score and the sequence coverage.

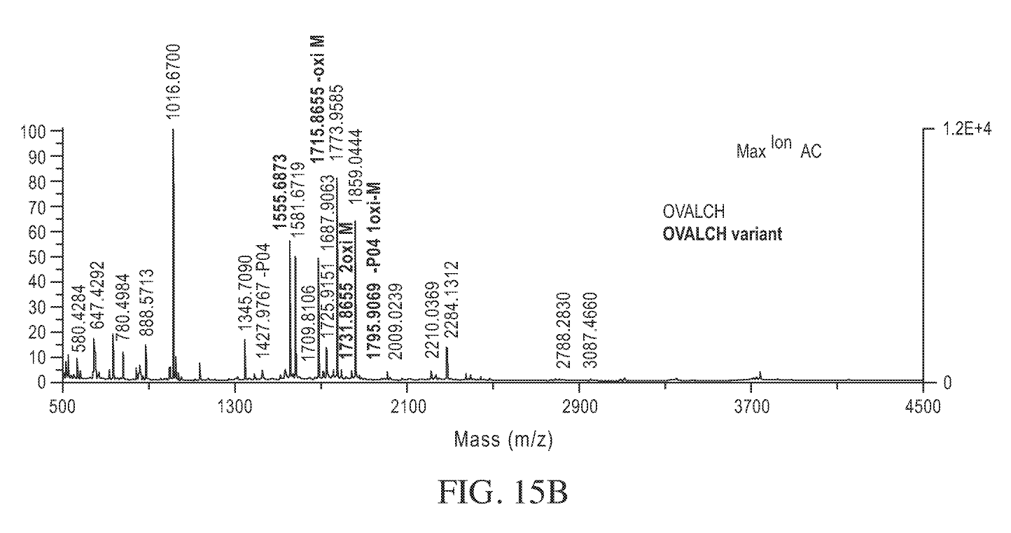

For all proteins studied, MaxIon AC silica resin yielded higher sequence coverage than conventional CHCA. MaxIon AC silica resin also yielded higher MASCOT scores for all proteins except ovalbumin, although MaxIon AC silica resin did yield higher sequence coverage. The reason is that the ovalbumin sample is not homogeneous (Sigma-Aldrich) and MaxIon “amplifies” the peak intensity of multiple lower-abundance ovalbumin isoforms (FIG. 15; isoforms are color coded to mass ions). Thus, although the sequence coverage for ovalbumin is higher, the increased number of “contaminant peaks” resulted in a lower confidence score by MASCOT, which searches for single accession numbers per spectrum.

TABLE 10
MASCOT SEQUENCE IDENTIFICATION CONFIDENCE
SCORE AND % SEQUENCE COVERAGE WITH OR
WITHOUT MAXION AC

| | MASCOT | Sequence | |
|---|---|---|---|
| Tryptic digest (amount) | score | coverage | Matrix |
| Ovalbumin (1 pmol) | 142 | 48% | CHCA |
| Ovalbumin (1 pmol) | 137 | 61% | MaxIon AC |
| Fetuin (1 pmol) | 54 | 16% | CHCA |
| Fetuin (1 pmol) | 120 | 26% | MaxIon AC |
| Myoglobin (1 pmol) | 86 | 46% | CHCA |
| Myoglobin (1 pmol) | 119 | 69% | MaxIon AC |
| Beta-Gal (400 fmol) | 430 | 60% | CHCA |
| Beta-Gal (400 fmol) | 507 | 66% | MaxIon AC |

### Example 20

### MaxIon AC: Effect of Silica on Signal-to-Noise Ratio in MALDI-TOF MS Using Complex Samples

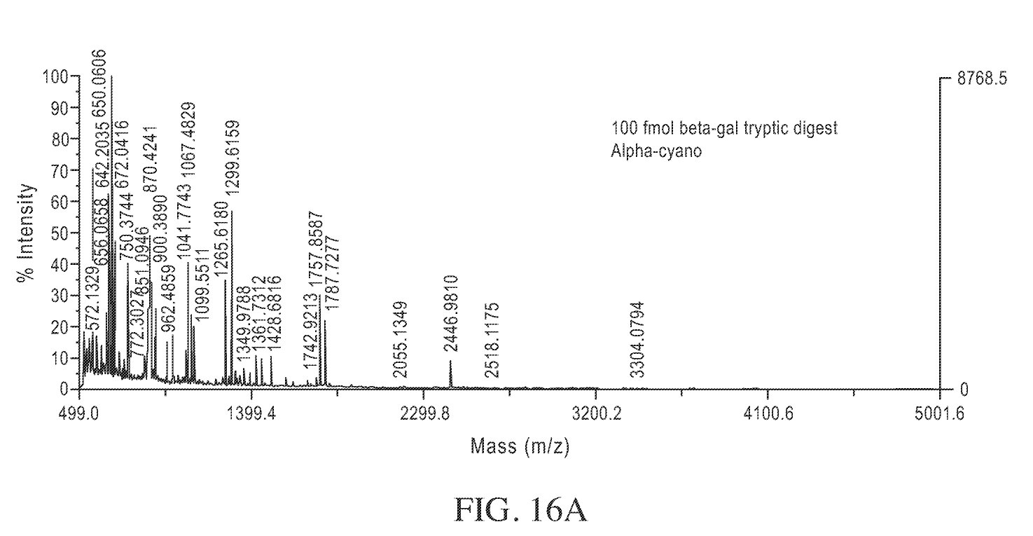

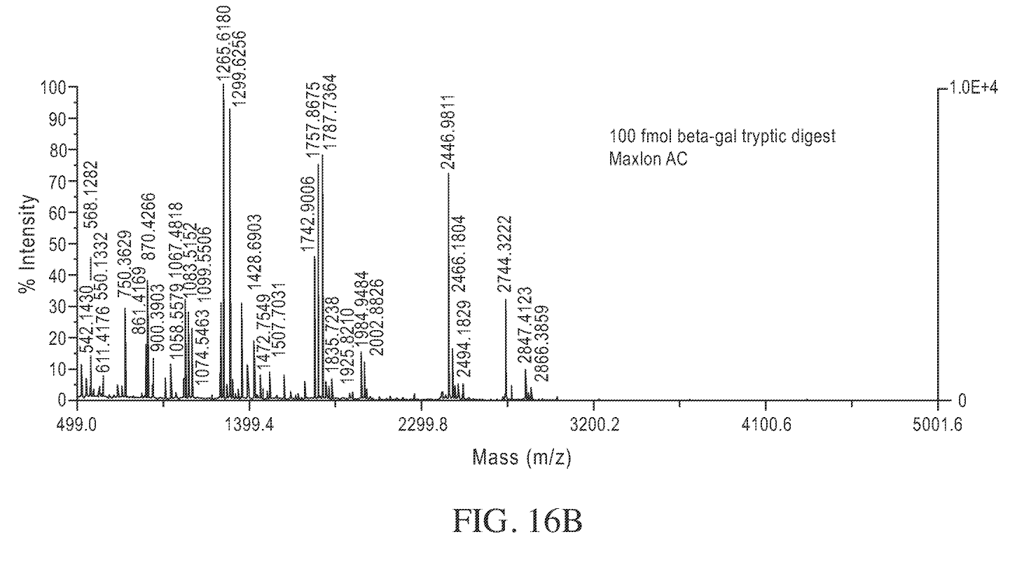

In order to determine if silica added to CHCA matrix could also improve the signal-to-noise of a sample from biological origin, which is typically more complex than a mixture of synthetic pure peptides, the following experiments were carried out. A tryptic digest of beta-galactosidase, which contains a range of peptides spanning a range of the hydrophobicity index, was used as a paradigmatic test biological sample since it. FIG. 16 shows MALDI-MS spectra of 100 fmol of digested beta-galactosidase spotted in typical alpha-cyanno (top) versus alpha-cyanno and the silica additive (bottom). The signal-to-noise of peaks corresponding to beta-galactosidase peptides was markedly improved, and the matrix background was notably suppressed, in the presence of silica. Notably, higher molecular weight mass-ions exhibit enhanced ionization in the presence of silica (mz/2446.98, m/z 2744.32, m/z 2847.41).

### Example 21

### MaxIon AC: Suppression of Salt Effects by Silica

The standards mix was analyzed in the presence of a high concentration of salt while the instrument low mass gate was inactivated, and the mass range increased to 10-5000 Da. The spectrum on the bottom left of FIG. 14 clearly shows how 500 mM NaCl induces the formation of matrix clusters that suppress the ionization of the standards across the spectrum. The spectrum on the bottom right of FIG. 14 shows that addition of silica, in most instances, reverses these salts effects and improves the overall spectral quality, although m/z 2932.6 remained suppressed.

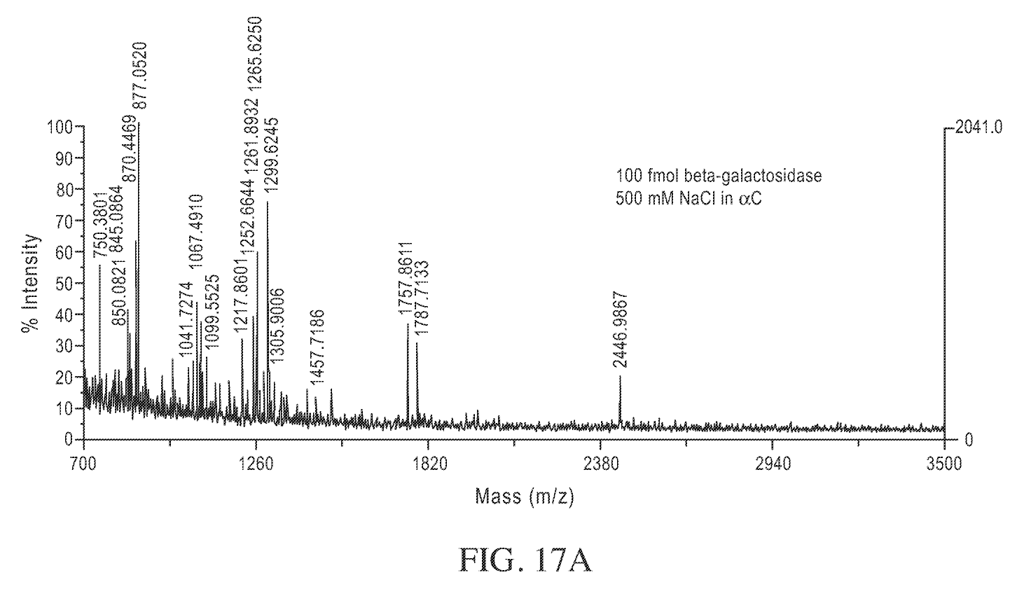

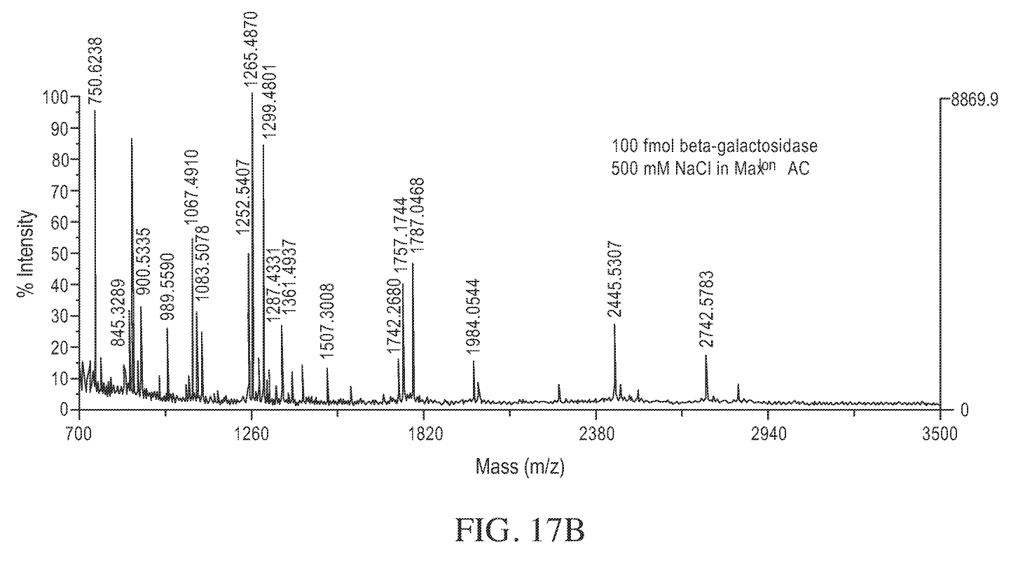

The experiment was repeated to determine the ability of MaxIon AC silica resin to relieve ionization suppression by high concentrations of salt. The same tryptic digest of beta-galactosidase was diluted into 500 mM NaCl (final digest concentration 100 fmol) and analyzed by MALDI-MS using conventional CHCA versus MaxIon AC silica resin. FIG. 17 illustrates the markedly improved spectral quality that MaxIon AC silica resin produces over conventional CHCA. The lower spectrum clearly shows the improved signal-to-noise where the total ion counts are approximately four fold higher than that in the CHCA spectrum. Further, the lower spectrum has a number of peaks corresponding to beta-galactosidase peptides that are suppressed by the high salt concentration in the top spectrum. The peaks from both spectra were analyzed by the database search application MASCOT (Matrix Science) and MaxIon AC silica resin yielded a score of 147 and sequence coverage of 37%. Conventional CHCA yielded only a MASCOT score of 118 and 17% sequence coverage (MASCOT scores >57 are statistically significant). Thus, even in the presence of high concentrations of salt, MaxIon AC silica resin can deliver greater signal-to-noise, which results in better sensitivity and protein identifications with improved confidence and sequence coverage.

### Example 22

### MaxIon AC: Other Compositions

Other compounds and compositions were tested for their ability to enhance MALDI-MS analysis in a manner similar to that of the silica resin used in the preceding experiments. Various parameters were identified that can be used, alone or in combination, to identify and characterize compositions having any or all of the desirable characteristics of silica as regards MALDI-MS analysis. For ease of discussion, such compounds and compositions are referred to herein as being “non-volatile matrix additives”, but it should be understood that phrase refers generally to compounds and compositions having one or more desirable characteristics of silica as regards MALDI-MS analysis and generally having the ability to enhance the quality of MS MALDI spectra under various conditions.

### Composition

The non-volatile matrix additive can be silica or a compound containing silica. Such compounds include without limitation silicon dioxide (SiO₂), silicon carbide, (SiC) and silicates.

As used herein, the term “silica” refers to a tetravalent nonmetallic element (Si) that occurs combined as the most abundant element next to oxygen in the earth's crust. Natural silicon dioxide (SiO₂) occurs in crystalline, amorphous and impure forms (quartz, opal and sand respectively).

Commercially available forms of silica that may be used to practice the invention include without limitation LiChrospher®, LiChroprep®, LiChroprep® and Purospher® RP-18 (registered trademarks of Merck KGaA, Darmstadt, Germany). LiChrosorb® is an irregular porous packing material manufactured in Germany by E. Merck. LiChrosorb® comprises porous irregular silica particles are finely classified in the 5 μm, 7 μm and 10 μm range. LiChrosorb® is available with different modifications, such as polar derivatives (Si60 and Si100), non-polar derivatives (RP-8, RP-18 and RP-select B), and derivatives of medium polarity (NH₂, CN and DIOL). LiChroprep® comprises porous irregular silica particles are finely classified in the 15-25 μm, 25-40 μm and 40-63 μm range. LiChrospher® (the 40 μm material is a.k.a. Superspher® in Europe) comprises particles that are more regular than LiChrosorb. LiChrospher® is available with different modifications, such as the polar modified derivatives LiChrospher® CN, LiChrospher® NH₂ and LiChrospher® DIOL, as well as LiChrospher® Si. Purospher® RP-18 is based upon a high purity, metal free silica. It is relatively chemically stable.

Silicates are arrangements of the elements silicon and oxygen with a wide variety of other elements (most common silicates are quartz and feldspars). The invention can be practiced with silicates other than silica if they are prepared in the proper form and/or ground to an appropriate particle size (see below).

A MS-compatible sorbent of the invention can be silica; alumina; titanium; tin; germanium oxide; an indium tin oxide; a metal oxide; a chloride; a sulfate; a phosphate; a carbonate; a fluoride; a polymer-based oxide, chloride, sulfate, carbonate, phosphate or fluoride; diatomaceous earth; graphite or activated charcoal; gold; or activated gold. The term “alumina” refers to various forms of aluminum oxide, including the naturally occurring corundum. Tin, titanium and alumina have been examined (data not shown) and, to some degree, all have the desirable characteristics of silica as regards MS-MALDI analysys, i.e., they reduce noise, reduce or eliminate adduction, provide for a greater density and more even distribution of crystals and co-crystals on a MALDI target plate, and the like.

### Sorbent Properties

The MS-compatible sorbents of the invention can be inorganic sorbents. Sorbents are insoluble, or partially or practically insoluble, materials or mixtures of materials used to recover liquids through the mechanism of absorption, or adsorption, or both. By “partially insoluble”, it is meant that the sorbent is at least 50% or about 50% insoluble in excess fluid, preferably 70% or about 70%, most preferably 80% or about 80% insoluble. A practically insoluble substance is at least about 85%, preferably 90% or about 90%, most preferably 95% or about 95% insoluble in excess fluid. An insoluble substance is at least about 98%, preferably 99% or about 99%, most preferably 100% or about 100% insoluble in excess fluid. Absorbents, in contrast, are materials that pick up and retain liquid distributed throughout their molecular structure causing the material to swell as much as 50% or about 50%, or more.

In addition to natural sorbents such as alumina and silica, synthetic sorbents may be used. These include polymer-based sorbents, such as Tenax-GC and Tenax-TA, which are available in various mesh sizes (20-35, 35-60, 60-80, and 80-100 microns, for example) from Scientific Instrument Services, Inc. (Ringoes, N.J.). The Tenax compositions comprise poly(2,6-diphenyl-1,4-phenylene oxide).

### Particle Shape and Size

The matrix additive can be provided in a variety of forms, typically as a colloid, such as a colloidal suspension, a resin or slurry. The additive can be in the form of at least partially suspended beads. The composition and particle size of any given non-volatile matrix additive will influence the choice of colloid. Particle size influences other factors as well. For example, powdered (fumed) silica disrupts crystal and co-crystal formation, presumably due to the extremely small particle size (<1 micron).

The concept of particle size encompasses several characteristics, including without limitation particle shape, mean particle size and particle size distribution. The particles can be spherical beads as well as more irregular particles.

The terms D10, D50, D90 and the like are used to evalauate particle size distribution and have the following meaning. When passed through a mesh or filter of a known pore size, D10 is the size of a pore through which 10% of the particles pass through (10% of the particles are smaller than the pore size); D50 is the point at which 50% of the particles are smaller; D90 is the point at which 90% of the particles are smaller; and so on.

As regards size distribution, gradation index (GI) is used to indicate the degree of uniformity for the distribution of particle sizes. By way of non-limiting example, the ratio of D90/D10 is used to evaluate GI herein, but other ratios can be established for any given field or application. When GI has a low value, the material has a uniform particle size distribution, whereas a high value indicates a wide range of particle sizes. In the non-volatile matrix additives of the invention, the GI (D90/D10) is ≦10 or about 10, preferably the GI≦5 or about 5, more preferably the GI≦3 about 3, and most preferably, the GI≦2.5 or about 2.5, or ≦2 or about 2.

Table 11 lists some forms of silica that meet such criteria and thus may be used to practice the invention. The LiChrosorb® compositions are commercially available from EMD Biochemicals/Calbiochem (San Diego, Calif.).

TABLE 11
PARTICULATE FORMS OF SILICA

| Silica Additive | Particle Size |
|---|---|
| Powdered Silica (“fumed silica”)* | 10-20 nm |
| Silica gel, | 40-63 μm |
| 230-400 mesh* | |
| Silica gel, | 63-200 μm |
| 70-230 mesh* | |
| Silica gel, | 75-150 μm |
| 100-200 mesh* | |
| Silica gel, | 75-250 μm |
| 60-200 mesh* | |
| Silica gel, | 75-650 μm |
| 28-200 mesh* | |
| Silica gel, | 150-250 μm |
| 60-100 mesh* | |
| Silica gel, | 200-500 μm |
| 35-70 mesh* | |
| Silica gel, | 250-500 μm |
| 35-60 mesh* | |
| LiChrosorb ® Si60 5 μm** | D10 = 3.5-4.2 μm |
| | D50 = 5.0-6.0 μm |
| | D90 = 8.0-10.8 μm |
| | GI = 10.8/4.2 = 2.57 |
| LiChrosorb ® 5 μm RP8** | D10 = 3.9-4.4 μm |
| | D50 = 5.7-6.5 μm |
| | D90 = 8.0-10.0 μm |
| | GI = 10/4.4 = 2.27 |
| LiChrosorb ® 5 μm RP-18** | D10 = 3.5-4.5 μm |
| | D50 = 5.5-6.5 μm |
| | D90 = 8.0-10.0 μm |
| | GI = 10/4.5 = 2.22 |
| LiChrosorb ® 5 μm RP-Select B** | D10 = 3.0-4.5 μm |
| | D50 = 4.5-6.5 μm |
| | D90 = 7.0-10.8 μm |
| | GI = 10.8/4.5 = 2.4 |
| LiChrosorb ® 5 μm DIOL** | D10 = 3.9-4.4 μm |
| | D50 = 5.7-6.5 μm |
| | D90 = 8.0-10.0 μm |
| | GI = 10/4.4 = 2.27 |
| LiChrosorb ® 10 μm RP18** | D10 = 6.5-8.5 μm |
| | D50 = 9.5-12.5 μm |
| | D90 = 13.0-17.0 μm |
| | GI = 2.00 |
| LiChrosorb ® 10 μm RP8** | D10 = 6.5-8.5 μm |
| | D50 = 9.5-12.5 μm |
| | D90 = 13.0-17.0 μm |
| | GI = 2.00 |
| LiChrosorb ® 10 μm RP18** | D10 = 6.5-8.5 μm |
| | D50 = 9.5-12.5 μm |
| | D90 = 13.0-17.0 μm |
| | GI = 2.00 |
| Silica Gel 60 RP-18** | 90% between 40 and 64 μm |

*Commercially available from, e.g., Sigma-Aldrich, St. Louis, MO.
**Commercially available from, e.g., Phenomenex, Inc., Torrance, CA.

### Example 23

### MaxIon SA: MES, MOPS and Related Compounds as MALDI Matrix Additives

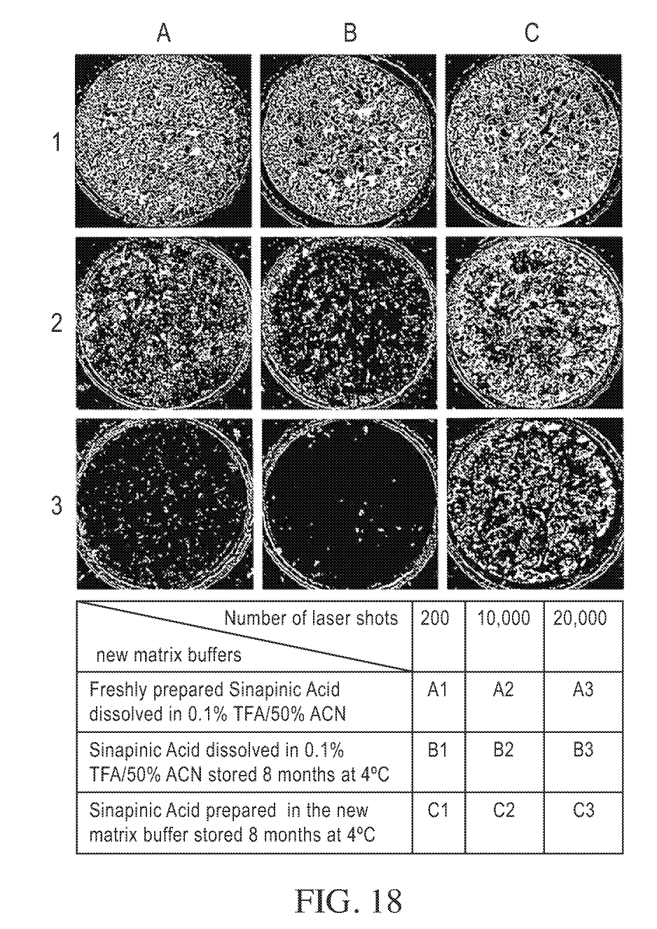

It was observed that intact protein samples dissolved in the buffer 2-(N-Morpholino)ethanesulfonic Acid (MES) produce co-crystals when co-mixed with the SA matrix for MALDI-MS analysis. An experiment was carried out to determine the ability of MES to resist laser ablation under MALDI-MS conditions. SA dissolved in 50% acetonitrile (ACN)/0.1% TFA was compared with SA dissolved in 50% ACN/0.1%TFA/40 mM MES. FIG. 18 shows images of 1 μL spots of SA dissolved in the absence or presence of MES. Even after 20,000 laser shots, the SA/MES sample (C3) displays marked resistance to laser ablation compared to SA spots without MES (A3 and B3). Thus, MES appears to stabilize the physical structure of the SA crystal under MALDI-MS conditions.

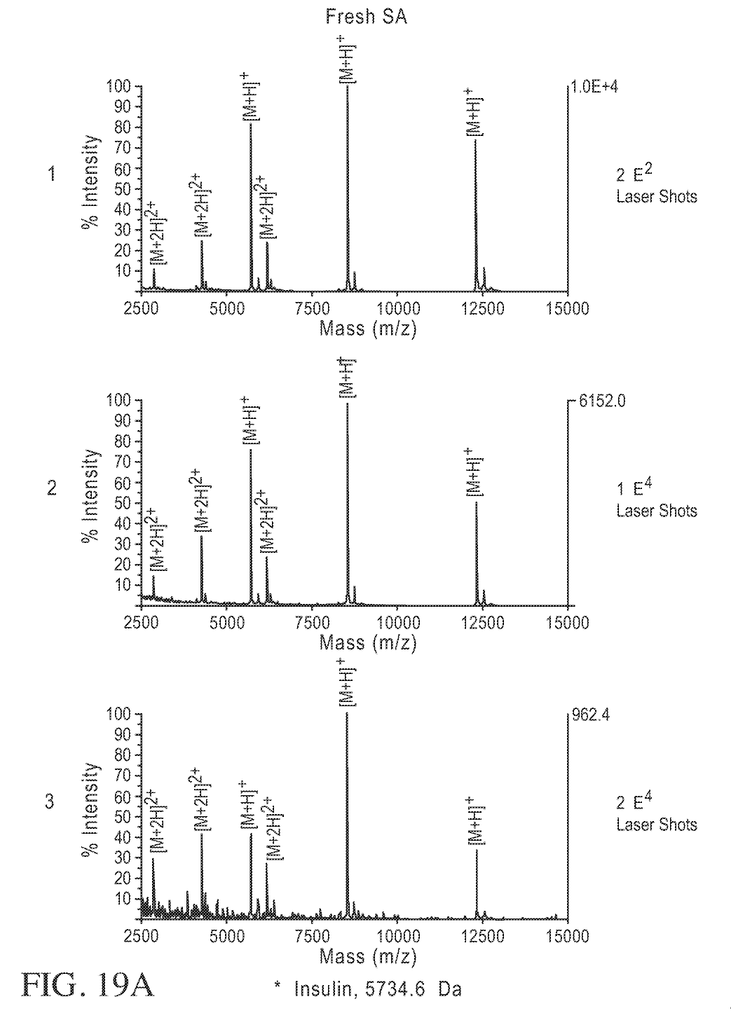

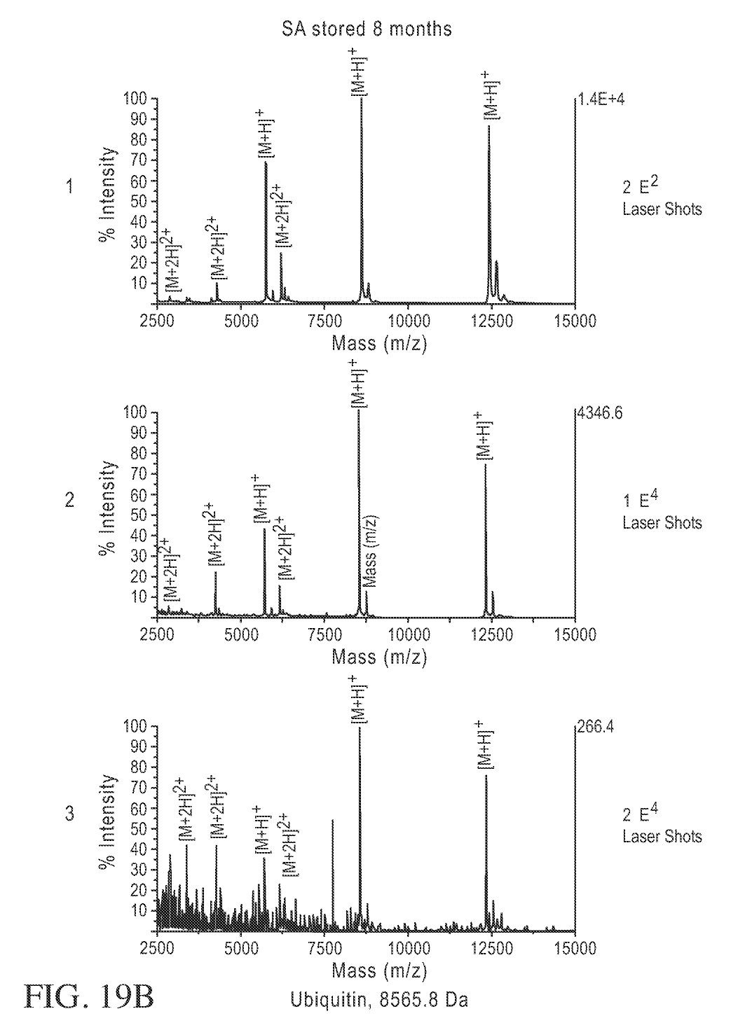

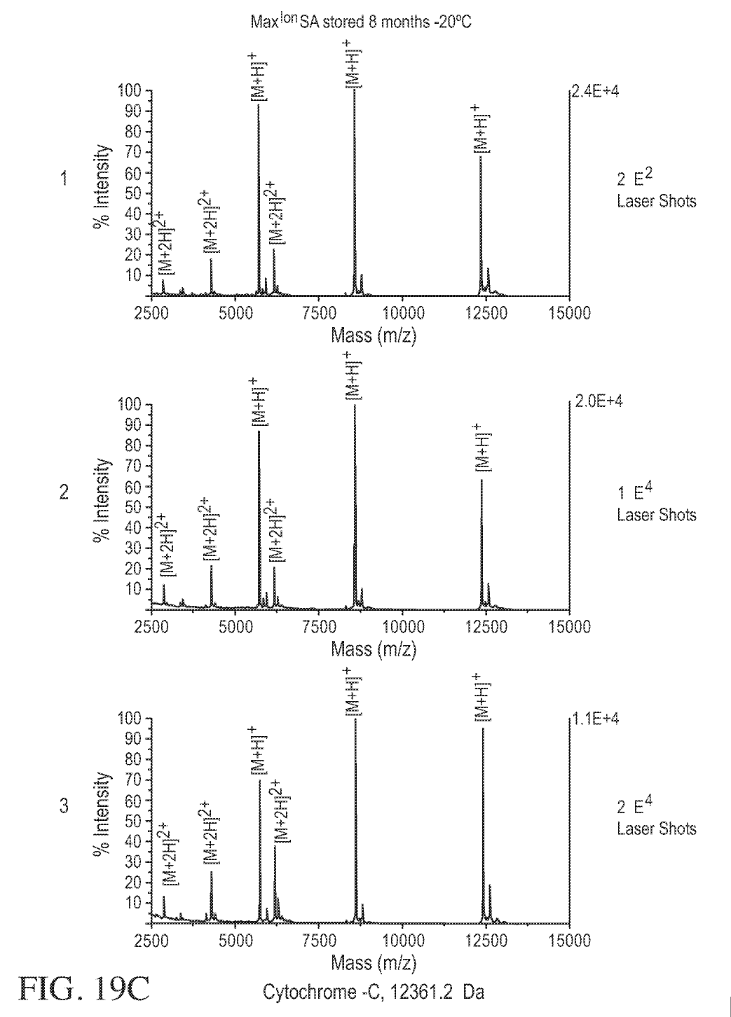

The experiment was repeated with co-spotting of a protein mix (insulin, ubiquitin, cytochrome-c) and using MALDI-MS spectral quality as an assay of the stability of SA/MES crystals. FIG. 19 illustrates how the signal-to-noise of the various protein analyte peaks diminishes with increasing number of laser shots for SA in the absence of MES (A1-A3, B1-B3). However, SA in the presence of MES was able to resist the loss in signal-to-noise as the number of laser shots increased (column C). Thus, the ability of MES to resist laser-induced crystal damage also lessens the loss of signal during increasing exposure to laser irradiation.

Experiments with the buffer MOPS (3-(N-Morpholino)propanesulfonic acid), which differs from MES only by an additional (—CH₂) moiety in the carbon chain between the morpholino and the sulfonate moieties, were also carried out. Although somewhat less pronounced than MES, MOPS also enhanced stability to laser-induced crystal damage.

### Example 24

### MaxIon SA: Effect of MES, MOPS and Related Compounds on Signal-to-Noise Ratio

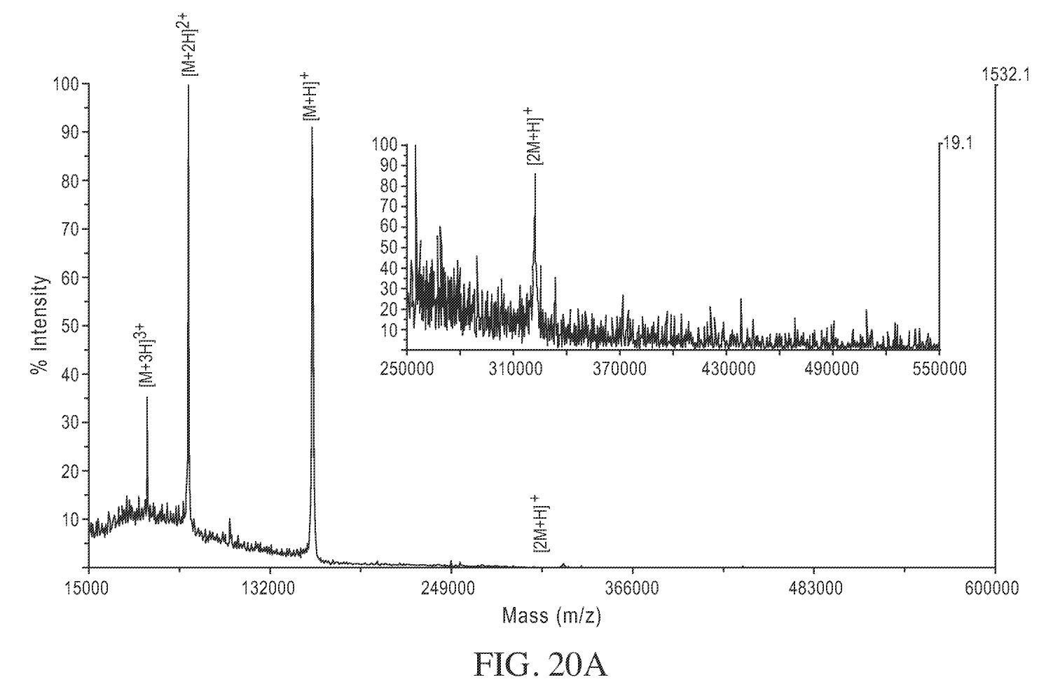

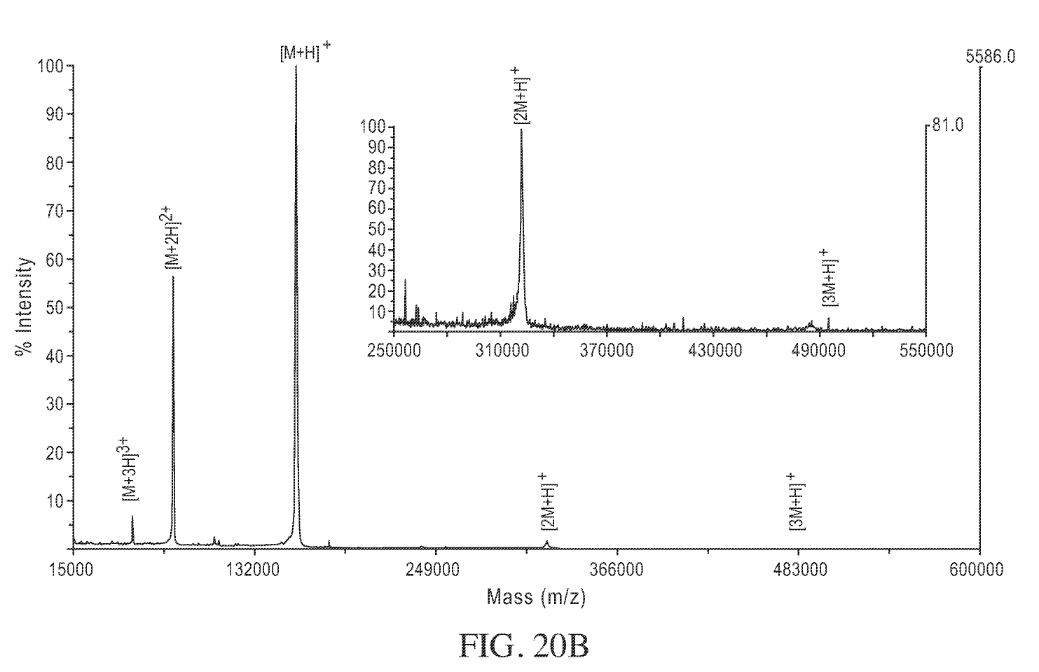

Detection of high molecular weight proteins by MALDI-TOF-MS can be especially challenging due to the inherent poor ionization efficiency. In order to detect these large proteins, higher laser intensities, longer acquisitions and more spectra are needed to sum and average spectra in order to maximize signal-to-noise. FIG. 20 illustrates an example of a MALDI-TOF analysis of a high molecular weight protein and how MaxIon SA can enhance the signal intensities of low abundance proteins. Spectrum A shows the prevalence of noise and the resulting perturbation of the baseline. Spectrum B shows how MaxIon SA can practically eliminate the baseline perturbation and markedly improve the signal-to-noise of the analyte peaks. Further, MaxIon SA allows the improved mass measurement of the dimer peak, and even the identification of the trimer (insets).

### Example 25

### MaxIon SA: Structures of MES, MOPS and Related Compounds

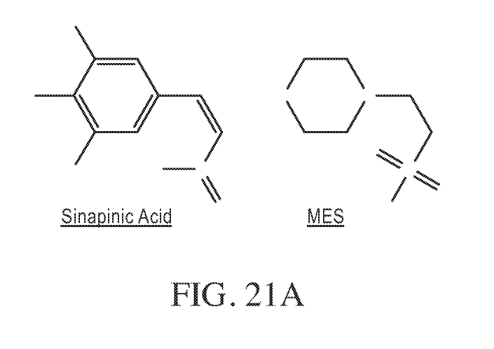

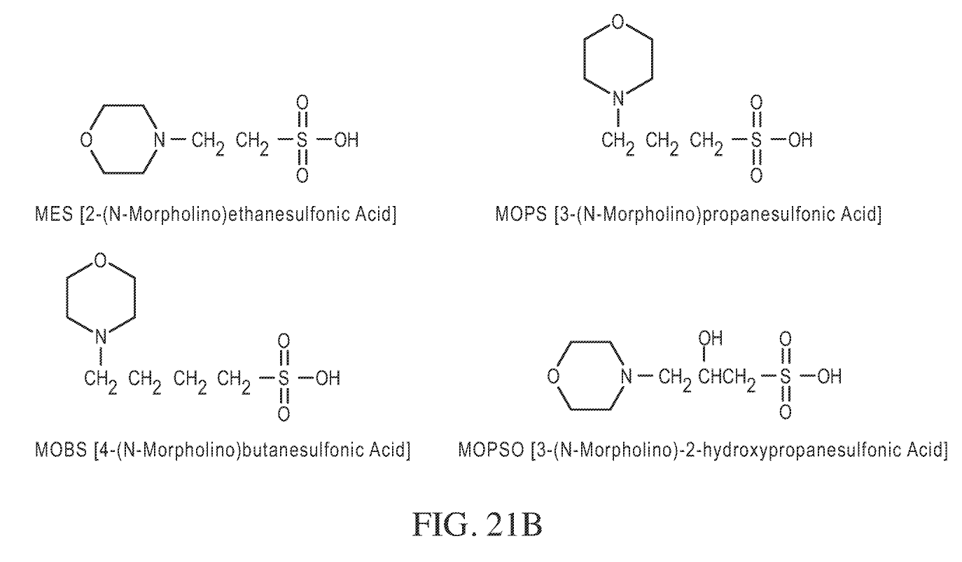

FIG. 21 shows the chemical structures of the MES (2-(N-Morpholino)ethanesulfonic Acid), MOPS (3-(N-Morpholino)propanesulfonic Acid), MOPSO (3-(N-Morpholino)-2-hydroxypropanesulfonic Acid) and MOBS (4-(N-Morpholino)butanesulfonic Acid) buffers. It is believed that these and other morpholino-sulfonic acids, as well as other related compounds, may also be useful as MALDI matrix additives.

One type of matrix additive of the invention has the structure

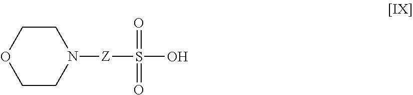

Wherein Z═[CH₂]ₐ—[CH—OH]_{b}—[CH₂]_{c}, and wherein:

a=0 to 25,

b=0 to 25,

c=0 to 25,

with the exception that, if b=0, a and c cannot both be 0.

By way of non-limiting example, in the structure of MES, a=2, b=0 and c=0; in MOPS, a=3, b=0 and c=0; in MOPSO a=1, b=1 and c=1; and in MOBS, a=4, b=0 and c=0. See FIG. 21 for further details.

### Example 26

### MaxIon Formulations

In some embodiments, the non-volatile MALDI matrix additives are comprised within a solution into which the MALDI matrix is dissolved or diluted. A MaxIon SA Matrix Diluent comprises, by way of non-limiting example,50% acetonitrile (v/v) (HPLC grade); 0.1% TFA (v/v) (HPLC grade); and 40 mM MES, in a final volume of 1.1 ml. An exemplary MaxIon AC Matrix Diluent comprises, by way of non-limiting example, 80% acetonitrile (v/v) (HPLC grade) and 0.05% TFA (v/v) (HPLC grade), in a final volume of 1.1 ml.

An exemplary 10× stock solution of MaxIon AC Resin comprises 20 mg of Lichrosorb Si60 (5 μm beads) silica. The MaxIon AC Resin Diluent comprises 99% acetonitrile (v/v) and 0.01% ammonium hydroxide (v/v) (ACS grade) in a final volume of 1.1 ml.

The CHCA matrix may be provided as, by way of non-limiting example, 2.2 mg of CHCA in a 1.5 ml eppendorf tube. This can be prepared by adding 100 μL of a solution of 22 mg/ml of CHCA in 100% methanol (HPLC grade) to a 1.5 ml eppendorf tube, and then drying to remove the menthol, e.g., by using a speed-vac with no heat.

The MaxIon AC Cation Sequestration Diluent (5×) is 500 mM NDSB in a final volume of 1.1 ml.

TABLE 12
MATERIALS AND SOURCES

| MATERIAL | SOURCE |
|---|---|
| Sinapinic acid | Sigma (a) |
| MES | Research Organics (b) |
| CHCA | Sigma (a) |
| Lichrosorb Si60 (5 μm) | EMD Biosciences (c) |
| NDSB | Calbiochem (d) |
| Acetonitrile | Sigma (a) |
| TFA | Pierce (e) |
| Ammonium Hydroxide | EMD Biosciences (c) |

(a) Sigma-Aldrich Corp. (St. Louis, MO).
(b) Research Organics (Cleveland, OH).
(c) EMD Biosciences, Inc. (San Diego, CA).
(d) Calbiochem ® is a brand of EMD Biosciences, Inc. (San Diego, CA).
(e) Pierce Biotechnology, Inc. (Rockford, IL).

### Example 27

### MaxIon Kits

In one aspect, a kit of the invention comprises MaxIon SA and/or MaxIon AC. Other kit components include without limitation:

(a) MALDI matrices (e.g., SA and CHCA);

(b) Standards as positive controls for the evaluation of the performance of the product, the spotting technique and the performance of the instrument;

(c) Instructions describing the matrix reconstitution in the diluents, the application of these matrices, and storage conditions; and

(d) Examples of MALDI-MS spectra of the included standards for use in troubleshooting and/or calibration.

At least 3 types of kits are contemplated by this aspect of the invention: MaxIon SA, MaxIon AC and MaxIon Complete.

An exemplary MaxIon SA kit, geared towards applications wherein SA is the MALDI matrix, contains five 1.5 ml eppendorff vials of Sinapinic Acid (20 mg), five 1.5 ml eppendorff vials of SA diluent (40 mM MES, 50% acetonitrile, 0.1% TFA), and one 0.5 ml eppendorff vial with InvitroMass LMW Calibrant mix 4 (Invitrogen).

An exemplary MaxIon AC kit, geared towards applications wherein CHCA is the MALDI matrix, contains five 1.5 ml eppendorff vials of CHCA (2.2 mg), five 1.5 ml eppendorff vials of CHCA diluent 1 (80% acetonitrile, 0.1% TFA), one 1.5 ml eppendorff vial of MaxIon AC Resin (20 mg of silica beads in 99% acetonitrile, 0.01% ammonium hydroxide), one 1.5 ml of Cation Sequestration Diluent (1 ml of 500 mM NDSB), and one 0.5 ml eppendorff tube of InvitroMass LMW Calibrant mix 2 (Invitrogen).

A MaxIon Complete kit, which can be used with a variety of MALDI matrices, contains the contents of the MaxIon SA and MaxIon AC kits.

Different calibrants can alternatively or additionally be included. These include without limitation the Invitromass™ calibrants from Invitrogen. The Invitromass™ Calibrant 1 set (500-1000 Da) comprises Bradykinin fragment (aa 1-5), Bradykinin fragment (aa 1-7) and Lys(-Des-Arg9, Leu8)-Bradykinin. The Invitromass™ Calibrant 2 set (1000-3000 Da) comprises Lys(-Des-Arg9, Leu8)-Bradykinin; ACTH fragment (aa 1-16); and ACTH fragment (aa 1-24). The Invitromass™ Calibrant 3 set (3000-6000 Da) comprises ACTH fragment (aa 1-24); ACTH fragment (aa 1-39); and Insulin. The Invitromass™ Calibrant 4 set (6000-12000 Da) comprises Insulin, Ubiquitin and Cytochrome C.

### Example 28

### Stability Studies

### MaxIon AC

During the course of the studies described in the sections above, we observed a time-dependent loss of performance of the MaxIon AC silica resin (data not shown). Specifically, MaxIon AC silica demonstrated a diminished ability to overcome the suppressive effects of sample salts. We hypothesized that the low pH in the matrix solution was promoting protonation of the SiO moieties. Generally, the MaxIon AC loses potency after 24 hrs in an acidic solution (0.05% TFA ˜pH 2.5). Preferably, the resin and CHCA are mixed immediately prior to use, and this solution is used within about 24, about 36, or preferably, 48 hours.

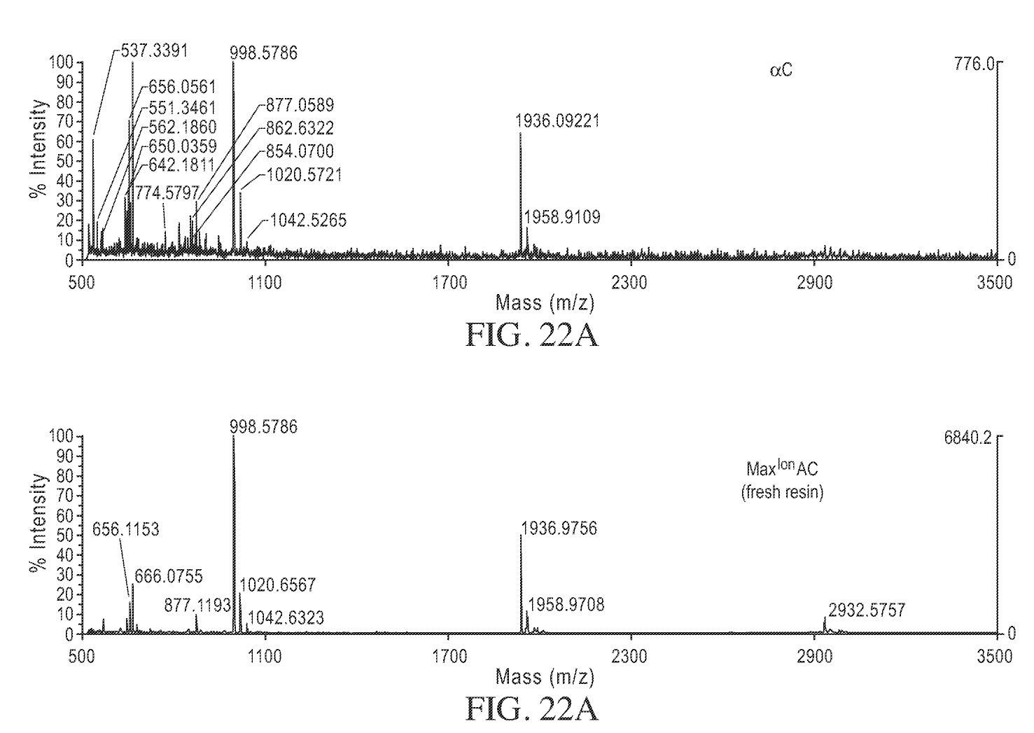

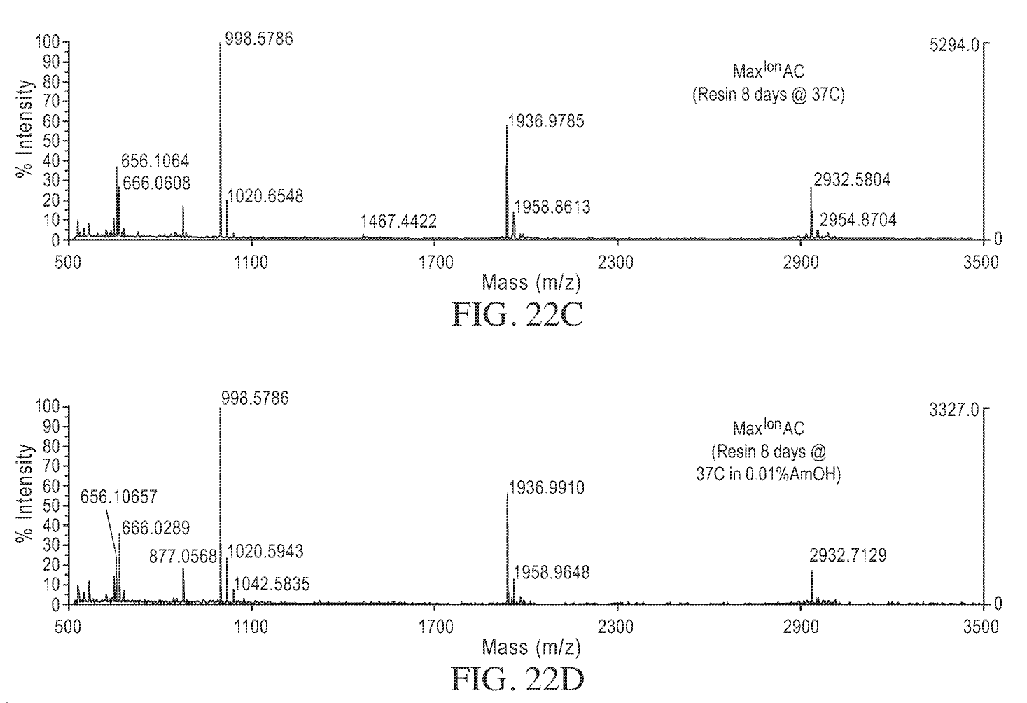

Studies were conducted to evaluate the stability of Lichrosorb Si60 when suspended as slurry in 99% Acetonitrile. Our concern was that the Lichrosorb Si60 silica resin would lose potency under storage conditions. Solutions of Lichrosorb Si60 (20 mg/nil) were made in 99% acetonitrile (v/v) with and without 0.01% ammonium hydroxide (AmOH). These solutions were then stored at 37° C. for 8 days. The InvitroMass LMW calibrant 2 was tested by MALDI-TOF-MS using fresh solutions of CHCA (2 mg/ml) with fresh silica resin (2 mg/ml) or stored silica resin. FIG. 22 shows MALDI-MS spectra of InvitroMass LMW Calibrant 2 (diluted 1:200 in 100 mM NaCl) in CHCA and CHCA mixed with silica under different storage conditions. The results suggest that even after 8 days at 37° C., the silica resin performs as well as freshly prepared resin. Interestingly, we did not note a marked performance enhancement of resin stored with acetonitrile fortified with 0.01% AmOH. However, we recommend that the Resin Diluent formulation should still include 0.01% AmOH to protect the resin from unforeseen changes in pH during storage conditions. Based on the stability studies described above, we extrapolate that the resin slurry is stable up to 8 months when stored at −20° C.

### Maxion SA

The reagents included in MaxIon SA are stable when stored at −20° C. for up to eight months post-production. It is recommended that once the SA matrix has been dissolved in the MaxIon SA Diluent, the solution is stable for up to 2 weeks when stored at 4° C.

MaxIon SA Diluent is 50% acetornitrile (HPLC grade) (v/v); 0.1% (v/v) TFA (HPLC grade); and 40 mM MES, in a final volume of 1.1 ml). A non-limiting example of HPLC grade acetonitrile is HPLC Grade >99.93% acetonitrile (Sigma-Aldrich Corp., St. Louis, Mo., catalog No. 270717). A non-limiting example of HPLC grade TFA is Trifluoroacetic Acid, Sequanal Grade (Pierce Biotechnology, Rockford, Ill., catalog No. 28904). A non-limiting example of MES is 2-(N-Morpholino)ethanesulfonic acid, monohydrate (Research Organics, Cleveland, Ohio, catalog No. 0113M).

### Example 29

### Automated Systems

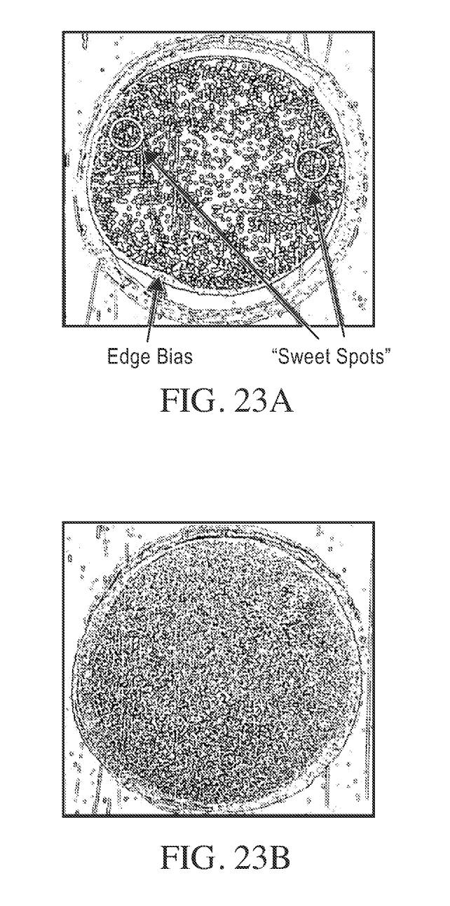

In order to examine the effects of silica on crystal (matrix) and co-crystal (analyte:matrix) homogeneity, the following experiments were carried out. Using silica as an additive Alpha cyano and alpha cyano:silica mixtures were prepared as described above. In initial experiments, a matrix solution, or a water blank, were co-spotted with 25 fmol of Beta-gal tryptic digest. However, due at least in part to light diffraction in the microscope, these did not show the heterogeneity in alpha-cyano alone that one can see using a MALDI instrument's camera and monitor. In order to enhance heterogeneity effects, 0.5 μL of matrix solution was co-spotted with 0.5 μL of 100 mM NaCl solution onto a stainless steel MALDI target plate. The results (FIG. 23) show that, without silica (A), crystals and co-crystals are not uniformly distributed and exhibit edge effects, thus requiring identification of “sweet spots” suitable for MALDI analysis. In contrast, the presence of silica (B) results in more (higher density) crystals and co-crystals, and also a more uniform distribution. The analyzable surface area of the plate shown on the right in FIG. 18 is much greater than the one on the left. Edge bias has been reduced or eliminated, and nearly the entire surface (as opposed to patches of the surface) of the plate consists of “sweet spots”.

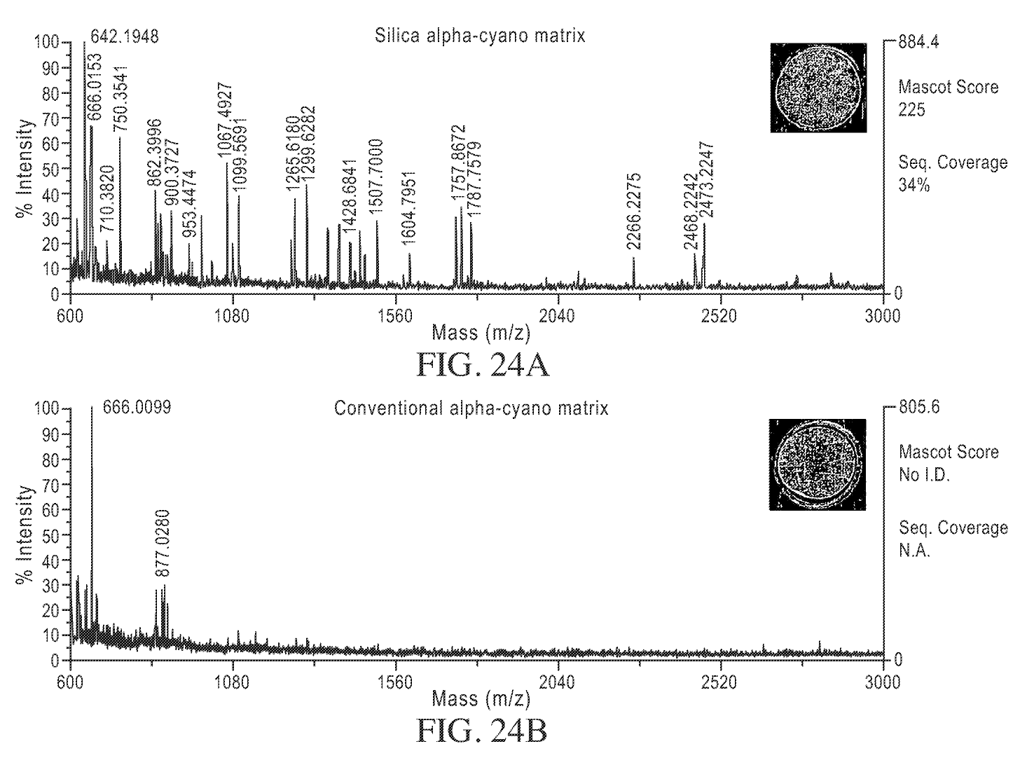

In order to examine the suitability of MALDI target plates prepared in this manner for automated MALDI scanning, such as might be used in HTS, the following experiment was carried out using the MALDI plates shown in FIG. 23. Rather than selecting and focusing “sweet spots” during acquisition and scanning of the plate lacking silica (A in FIG. 23), the laser's target point was moved about randomly on both plates. The resultant spectra (FIG. 24) show that, under these conditions, only the targets prepared with silica yield useful spectra. Indeed, the spectrum of the sample without silica is not even sufficient to result in identification of the protein analyte in the absence of targeting to sweet spots on the target plate.

### Example 30

### Peptide Identification Using an MS-Compatible Solubilizer with IPG Strip Separation and MS

Separation of peptides by immobilized pH gradients (IPG) is an effective method of sample fractionation and provides valuable pI data for sequence identification. Combining pI fractionation with LC-MS in bottom-up analysis of complex peptide mixtures can be used to validate peptide sequence and enhance sequence coverage of hydrophobic proteins.

Cytochrome P450 and nAChR were digested with trypsin (Promega) or endoproteinase AspN (Roche) overnight in the presence of an MS-compatible solubilizer blend of 25 mM NDSB-201, 25 mM NDSB-256, 0.22 mM SB-14, 25 mM Ammonium bicarbonate, pH 7.8. 5 ug of the digested samples were loaded directly on 3-10 NL IPG strips (Invitrogen) in addition to a 1 ug aliquot of myoglobin. After electrophoretic focusing, the strips were cut into 8 equal pieces and peptides were extracted using 5% TFA and 50% ACN/2.5% TFA. The extracted peptides were dried using speed vac (Savant) and were reconstituted in 20% ACN/2.5% FA. The samples were then analyzed with both 4700 MALDI-TOF/TOF (Applied Biosystems) and Q-TOF LC-ESI/MS (Waters). The data were analyzed by GPS explorer (Applied Biosystems), Mascot Distiller (Matrix Sciences) sequence database search and GPMAW 6.0 (ChemSW).

High recovery yields of digested peptides from IPG strips were obtained and thus we were able to analyze the same samples in parallel using the Q-TOF LC-ESI/MS and 4700 MALDI-TOF/TOF via LC separation using automated spotting. Electrophoretic migration of peptides exhibited good reproducibility between multiple separations which allowed for assignment of peptides into pI ‘bins’. These bins represent a pI range that is ⅛^{th} of the nonlinear 3 to 10 pH range of the strip.

Sequence assignments were made based on 1) the exact mass of the peptide, and 2) by comparing agreement between the predicted pI and the known pI of a myoglobin proteolytic fragment co-migrating within the same pI bin. We confirmed our sequence identifications by MS/MS analysis to determine if sequence assignments by our method were successful. We found that having exact mass and assigning peptides to a narrow pI range was sufficient to successfully identify peptide sequences. This method can be used to enhance sequence coverage of hydrophobic proteins, especially for the purpose of detecting membrane-spanning protein segments.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following claims.

What is claimed is:

1. A MS-compatible solubilizer comprising one or more components selected from the group consisting of
a. one or more MS-compatible detergents, wherein at least one of said MS-compatible detergents is at a concentration that is at least about 75% of its CMC; and
b. one or more MS-compatible non-detergent surfactants,
wherein an effective amount of said MS-compatible solubilizer has one or more characteristics selected from the group consisting of
i. increasing the signal-to-noise ratio by at least about 5%;
ii. decreasing by at least about 5% signals resulting from one or more adduct cluster peaks of a molecule that forms adduct with ions;
iii. increasing the analyzable surface area by at least about 1%;
iv. improving the solubility of an analyte by at least about 5% during one or more sample processing procedures;
v. improving the solubility of an analyte by at least about 5% in a composition comprising a matrix; and
vi. improving the stability of an analyte:matrix crystal by at least about 5%.

2. The MS-compatible solubilizer of claim 1, comprising one or more MS-compatible non-detergent surfactants and one or more MS-compatible detergents, wherein at least one of said one or more MS-compatible detergents is at a concentration that is at least about 75% of its CMC.

3. The MS-compatible solubilizer of claim 1, comprising one or more MS-compatible non-detergent surfactants and one or more MS-compatible detergents, wherein at least one of said one or more MS-compatible detergents is at a concentration that is greater than or equal to its CMC.

4. The MS-compatible solubilizer of claim 1, comprising two or more MS-compatible detergents, wherein each detergent is at a concentration that is at least about 75% of its respective CMC.

5. The MS-compatible solubilizer of claim 1, comprising two or more detergents, wherein each detergent is at a concentration that is greater than or equal to its respective CMC.

6. The MS-compatible solubilizer of claim 1, wherein an effective amount of said MS-compatible solubilizer increases the signal-to-noise ratio by at least about 5%.

7. The MS-compatible solubilizer of claim 1, wherein an effective amount of said MS-compatible solubilizer increases the signal-to-noise ratio by at least about twofold.

8. The MS-compatible solubilizer of claim 1, wherein an effective amount of said MS-compatible solubilizer increases the analyzable surface area by at least about 1%.

9. The MS-compatible solubilizer of claim 1, wherein an effective amount of said MS-compatible solubilizer increases the analyzable surface area by at least about 10%.

10. The MS-compatible solubilizer of claim 1, wherein an effective amount of said MS-compatible solubilizer improves the solubility of an analyte by at least about 5% during one or more sample processing procedures.

11. The MS-compatible solubilizer of claim 1, wherein an effective amount of said MS-compatible solubilizer improves the solubility of an analyte by at least about twofold during one or more sample processing procedures.

12. The MS-compatible solubilizer of claim 11, wherein said one or more sample processing procedures is selected from the group consisting of
a. analyte isolation;
b. sample processing;
c. mixing of the analyte, matrix and optional additives;
d. co-precipitation of analyte:matrix to produce analyte:matrix crystals
e. deposition of analyte:matrix crystal onto a MS target surface; and
f. washing analyte:matrix crystals in situ.

13. The MS-compatible solubilizer of claim 1, wherein an effective amount of said MS-compatible solubilizer improves the solubility of an analyte by at least about 5% in a composition comprising a matrix.

14. The MS-compatible solubilizer of claim 1, wherein an effective amount of said MS-compatible solubilizer improves the solubility of an analyte by at least about twofold in a composition comprising a matrix.

15. The MS-compatible solubilizer of claim 1, wherein an effective amount of said MS-compatible solubilizer improves the stability of an analyte:matrix crystal by at least about 5%.

16. The MS-compatible solubilizer of claim 1, wherein an effective amount of said MS-compatible solubilizer improves the stability of an analyte:matrix crystal by at least about twofold.

17. The MS-compatible solubilizer of claim 1, comprising one or more MS-compatible detergents selected from the group consisting of ASB-C8Ø, Octyl-beta-D-1-thioglucopyranoside, n-Dodecanoylsucrose, and SB14.

18. The MS-compatible solubilizer of claim 1, comprising ASB-C8Ø, Octyl-beta-D-1-thioglucopyranoside, n-Dodecanoylsucrose, and SB14.

19. The MS-compatible solubilizer of claim 17, wherein said MS-compatible solubilizer comprises ASB-C8Ø.

20. The MS-compatible solubilizer of claim 19, wherein the concentration of ASB-C8Ø is from about 0.01 mM to about 0.5 mM.

21. The MS-compatible solubilizer of claim 19, wherein the concentration of ASB-C8Ø is about 0.025 mM.

22. The MS-compatible solubilizer of claim 17, comprising Octyl-beta-D-1-thioglucopyranoside.

23. The MS-compatible solubilizer of claim 22, wherein the concentration of Octyl-beta-D-1-thioglucopyranoside is from about 1 to about 50 mM.

24. The MS-compatible solubilizer of claim 22, wherein the concentration of Octyl-beta-D-1-thioglucopyranoside is about 10 mM.

25. The MS-compatible solubilizer of claim 17, comprising n-Dodecanoylsucrose.

26. The MS-compatible solubilizer of claim 25, wherein the concentration of n-Dodecanoylsucrose is from about 0.1 to about 10 mM.

27. The MS-compatible solubilizer of claim 25, wherein the concentration of n-Dodecanoylsucrose is about 0.75 mM.

28. The MS-compatible solubilizer of claim 17, comprising SB14.

29. The MS-compatible solubilizer of claim 28, wherein the concentration of SB14 is from about 0.05 to about 1. mM.

30. The MS-compatible solubilizer of claim 28, wherein the concentration of SB14 is about 0.2 mM.

31. The MS-compatible solubilizer of claim 1, comprising one or more compounds selected from the group consisting of an alkyl glycoside, a sulfobetaine, and a bile acid.

32. The MS-compatible solubilizer of claim 1, wherein said MS-compatible solubilizer comprise one or more organic co-additives.

33. The MS-compatible solubilizer of claim 32, further comprising one or more co-additives are selected from the group consisting of phospholipids, fatty acids, steroid compounds and organic solvents.

34. The MS-compatible solubilizer of claim 1, wherein at least one of the one or more MS-compatible detergent is an alkyl glycoside.

35. The MS-compatible solubilizer of claim 34, wherein the MS-compatible detergent is an alkyl glycoside having the structure
R—Z—(CH₂)ₓ—CH₃
wherein:
Z can be O, S, Cl, I, Fl, Se, Br;
x=1-20;
when R=glucose, x=1-8; and
when R=maltose, x=1-11.

36. The MS-compatible solubilizer of claim 34, wherein at least one of the one or more MS-compatible detergent is an alkyl glycoside selected from the group consisting of n-ethyl-beta-D-glucopyranoside, n-propyl-beta-D-glucopyranoside, n-tetryl-beta-D-glucopyranoside, n-pentyl-beta-D-glucopyranoside, n-hexyl-beta-D-glucopyranoside, n-heptyl-beta-D-glucopyranoside, n-octyl-beta-D-glucopyranoside, n-nonyl-beta-D-glucopyranoside, n-ethyl-beta-D-maltoside, n-propyl-beta-D-maltoside, n-tetryl-beta-D-maltoside, n-pentyl-beta-D-maltoside, n-hexyl-beta-D-maltoside, n-heptyl-beta-D-maltoside, n-octyl-beta-D-maltoside, n-nonyl-beta-D-maltoside, n-decyl-beta-D-maltoside, n-monodecyl-beta-D-maltoside n-dodecyl-beta-D-maltoside, octyl-beta-D-1-thioglucopyranoside, and n-dodeconoylsucrose.

37. The MS-compatible solubilizer of claim 1, wherein the MS-compatible detergent is a sulfobetaine.

38. The MS-compatible solubilizer of claim 37, wherein the MS-compatible detergent is a sulfobetaine having the structure 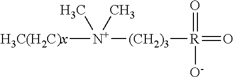
wherein:
R can be S, P or C; and
x can be 1-20.

39. The MS-compatible solubilizer of claim 38, wherein R is S.

40. The MS-compatible solubilizer of claim 38, wherein the MS-compatible detergent is selected from the group consisting of Zwittergent 3-08, Zwittergent 3-10. Zwittergent 3-12, Zwittergent 3-14, and Zwittergent 3-16.

41. The MS-compatible solubilizer of claim 1, wherein the MS-compatible detergent is a bile acid.

42. The MS-compatible solubilizer of claim 40, wherein the MS-compatible detergent is a bile acid having the structure 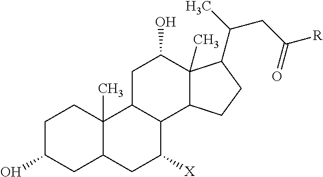
wherein:
R is a non-detergent sulfobetaine; and
X can be H or OH.

43. The MS-compatible solubilizer of claim 1, comprising one or ore MS-compatible non-detergent surfactants, wherein at least one of the one or more MS-compatible non-detergent surfactants is a non-detergent sulfobetaine.

44. The MS-compatible solubilizer of claim 42 or 43, wherein said non-detergent sulfobetaine has the structure 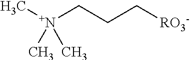
wherein:
R is S, P or C.

45. The MS-compatible solubilizer of claim 44, wherein R is S.

46. The MS-compatible solubilizer of claim 42 or 43, wherein said non-detergent sulfobetaine has the structure 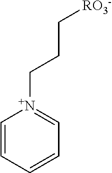
wherein:
R is S, P or C.

47. The MS-compatible solubilizer of claim 46, wherein R is S.

48. The MS-compatible solubilizer of claim 42 or 43, wherein said non-detergent sulfobetaine has the structure 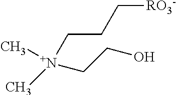
wherein:
R is S, P or C.

49. The MS-compatible solubilizer of claim 48, wherein R is S.

50. MS-compatible solubilizer of claim 42 or 43, wherein said non-detergent sulfobetaine has the structure 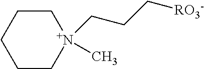
wherein:
R is S, P or C.

51. The MS-compatible solubilizer of claim 50, wherein R is S.

52. The MS-compatible solubilizer of claim 42 or 43, wherein said non-detergent sulfobetaine has the structure 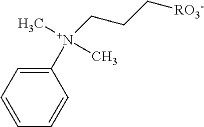
wherein:
R is S, P or C.

53. The MS-compatible solubilizer of claim 52, wherein R is S.

54. The MS-compatible solubilizer of claim 43, wherein said non-detergent sulfobetaine is selected from the group consisting of NDSB-195, NDSB-256, NDSB-201, NDSB-211, NDSB-221, NDSB-256 and sulfates, phosphates, and carbonates thereof.

55. The MS-compatible solubilizer of claim 1, wherein the MS-compatible detergent a Rabilloud detergent variant.

56. The MS-compatible solubilizer of claim 55, wherein said Rabilloud detergent variant has the structure 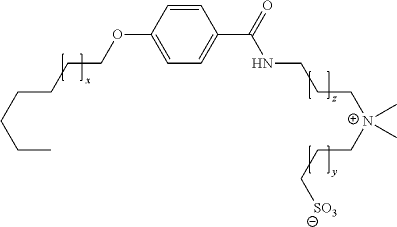
wherein:
x=0-25;
y=0-25; and
z=0-25.

57. The MS-compatible solubilizer of claim 55, wherein x=0-10; y=0-10; and z=0-10,

58. The MS-compatible solubilizer of claim 55, wherein x=0-5; y=0-3; and z=0-3.

59. An MS-compatible solubilizer, comprising ASB-C8Ø; Octyl-beta-D-1-thioglucopyranoside; n-Dodecanoylsucrose; and SB14.

60. The solution of claim 59, wherein the concentration of ASB-C8Ø is from about 0.01 to about 0.5 mM; the concentration of Octyl-beta-D-1-thioglucopyranoside from about 1 to about 50 mM; the concentration of n-Dodecanoylsucrose is from about 0.1 to about 10 mM; and the concentration of SB14 is from about 0.05 to about 1 mM.

61. The solution of claim 59, wherein the concentration of ASB-C8Ø is from about 0.025 mM; the concentration of Octyl-beta-D-1-thioglucopyranoside is about 10 mM; and the concentration of n-Dodecanoylsucrose is about 0.76 mM; and the concentration of SB14 is about 0.2 mM.

62. A stock solution of MS-compatible solubilizer that can be diluted from 2- to 1,000-fold to yield a solution comprising ASB-C8Ø at a final concentration of 0.025 mM; Octyl-beta-D-1-thioglucopyranoside at a final concentration of 10 mM; n-Dodecanoylsucrose at a final concentration of 0.76 mM; and SB14 at a final concentration of 0.2 mM.

63. A 5× stock solution of a MS-compatible solubilizer, comprising ASB-C8Ø at 0.125 mM; Octyl-beta-D-1-thioglucopyranoside at 50 mM; n-Dodecanoylsucrose at 3.8 mM; and SB14 at 1 mM.

64. A kit comprising a stock solution of MS-compatible solubilizer that can be diluted from 2- to 1,000-fold to yield a solution comprising ASB-C8Ø at a final concentration of 0.025 mM; Octyl-beta-D-1-thioglucopyranoside at a final concentration of 10 mM; n-Dodecanoylsucrose at a final concentration of 0.76 mM; and SB14 at a final concentration of 0.2 mM; and at least one MS standard or calibrant.

65. A stock solution of MS-compatible solubilizer that can be diluted from 2 to 1,000-fold to yield a solution comprising NDSB-201 at from about 25 mM to about 50 mM; NDSB-256 at from about 25 mM to about 50 mM; SB14 at from about 0.22 mM to about 0.5 mM; and ammonium bicarbonate, pH 7.8, at from about 10 to about 50 mM.

66. A 5× stock solution of a MS-compatible solubilizer, comprising NDSB-201 at 125 mM; NDSB-256 at 125 mM; SB14 at 1.1 mM; and ammonium bicarbonate, pH 7.8, at 125 mM.

67. A kit comprising a stock solution of MS-compatible solubilizer that can be diluted from 2- to 1,000-fold to yield a solution comprising NDSB-201 at 25 mM; NDSB-256 at 25 mM; SB14 at 0.22 mM; and ammonium bicarbonate, pH 7.8, at 25 mM;
and at least one MS standard or calibrant.

68. A method of mass spectrometry, comprising mixing a sample with the stock solution of claim 62 or 65 and performing mass spectrometry on the sample.

69. The method of claim 68, wherein said mass spectrometry is selected from the group consisting of APCI MS, ESI MS, GC MS. MALDI-TOF MS, LC/MS, LC/MS/MS, and MS/MS.

70. A method of liquid chromatography, comprising mixing a sample with the stock solution of claim 65 and subjecting the sample to liquid chromatography.

71. A composition comprising a MALDI matrix and an effective amount of one or more MALDI matrix additives, wherein an effective amount of said one or more additives has one or more characteristics selected from the group consisting of
a. increasing the signal-to-noise ratio by at least about 5%;
b. increasing by at least about 5% one or more adduct cluster peaks of a molecule that forms adduct with ions;
c. increasing the stability of an analyte:matrix crystal by at least about 5%;
d. diffracting and/or reflecting the incident laser beam in a MALDI matrix comprising one or more additives to a degree sufficient to alter the fluence by at least about 1%;
e. diffracting and/or reflecting the incident laser beam in a MALDI matrix comprising one or more additives to a degree sufficient to alter the fluence at least about 10 Joules/square centimeter; and
f. increasing, by at least about 1%, the amount of energy that is absorbed by a MALDI matrix.

72. The composition of claim 71, wherein an effective amount of said MS-compatible matrix additive increases the signal-to-noise ratio by at least about 5%.

73. The composition of claim 72, wherein an effective amount of said MS-compatible matrix additive increases the signal-to-noise ratio by at least about twofold.

74. The composition of claim 71, further comprising one or more ion-sequestering molecules.

75. The composition of claim 74, wherein said one or more ion-sequestering molecules is selected from the group consisting of sulfonates and zwitterionic surfactants.

76. The composition of claim 74, wherein said one or more ion-sequestering molecules are introduced to the MALDI sample as colloids.

77. The composition of claim 71, wherein an effective amount of said one or more MALDI matrix additives diminishes by at least about 5% one or more adduct cluster peaks of a molecule that forms adduct with ions.

78. The composition of claim 77, wherein said ions are cations.

79. The composition of claim 78, wherein said cations are monovalent cations.

80. The composition of claim 77, wherein the M-1 adduct cluster peak of a molecule that forms adduct with ions is decreased by at least about 50% in the presence of an effective amount of said MALDI matrix additive.

81. The composition of claim 80, wherein the M-1 adduct cluster peak of a molecule that forms adducts with ions is decreased by at least about 95% in the presence of an effective amount of said MALDI matrix additive.

82. The composition of claim of claim 71, wherein said molecule that forms adduct with ions is bradykinin.

83. The composition of claim 71, wherein said MALDI matrix additive comprises one or more MS-compatible sorbents.

84. The composition of claim 83, wherein said one or more MS-compatible sorbents is selected from the group consisting of silica; alumina; titanium; tin; germanium oxide; an indium tin oxide; a metal oxide; a chloride; a sulfate; a phosphate; a carbonate; a fluoride; a polymer-based oxide, chloride, sulfate, carbonate, phosphate or fluoride; diatomaceous earth; graphite or activated charcoal; gold; and activated gold.

85. The composition of claim 83, wherein said one or more MS-compatible sorbents is a resin.

86. The composition of claim 85, wherein said resin is selected from the group consisting of LiChrosorb®, LiChrospher®, LiChroprep®, LiChroprep® and Purospher®.

87. The composition of claim 85, wherein said resin is selected from the group consisting of LiChrosorb® 5 μm, 5 μm RP8, 5 μm RP18, LiChrosorb® 5 μm RP-Select B, LiChrosorb® 5 μm DIOL, LiChrosorb® 10 μm RP18, LiChrosorb® 10 μm RP8, LiChrosorb® 10 μm RP18, LiChrosorb® 5 μm Si60 and Silica Gel 60 RP-18.

88. The composition of claim 83, wherein said one or more MS-compatible sorbents is a composition comprising particles.

89. The composition of claim 88, wherein said particles comprise silica.

90. The composition of claim 88, wherein said composition comprising particles has a D90/D10≦about 2.0.

91. The composition of claim 88, wherein said particles have at least one dimension ≧1 microns.

92. The composition of claim 88, wherein said particles are irregular.

93. The composition of claim 88, wherein said particles are spherical.

94. The composition of claim 83, wherein said MS-compatible sorbents are non-volatile.

95. The composition of claim 71, wherein said MALDI matrix additive comprises one or more MS-compatible buffers.

96. The composition of claim 95, wherein said MS-compatible buffer is a morpholino-sulfonic acid.

97. The composition of claim 95, wherein said MS-compatible buffer is selected from the group consisting of MES, MOBS, MOPS, and MOPSO.

98. The composition of claim 95, wherein said MS-compatible buffer has the structure 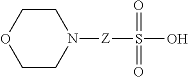
Wherein Z═[CH₂]ₐ—[CH—OH]_{b}—[CH₂]_{c},
and wherein:
a=0 to 25,
b=0 to 25,
c=0 to 25,
with the exception that, if b=0, a and c cannot both be 0.

99. A MALDI matrix diluent comprising a MS-compatible buffer.

100. A MALDI matrix diluent comprising 50% acetonitrile (v/v) (HPLC grade); 0.1% TFA (v/v) (HPLC grade); and 40 mM MES, in a final volume of 1.1 ml.

101. A method of preparing analyte:matrix crystals of an analyte for MALDI MS analysis, said method comprising
a. contacting said analyte with
i. a MALDI matrix; and
ii. one or more MALDI matrix additives selected from the group consisting of
(a) a MS-compatible solubilizer,
(b) a MS-compatible sorbent, and
(c) a MS-compatible buffer; and
b. co-precipitating said analyte with said MALDI matrix,
thus generating analyte:matrix crystals for MALDI MS analysis.

102. A method of obtaining a MALDI MS spectrum of an analyte, said method comprising
a. contacting an analyte with, in either order or in combination,
i. a MALDI matrix; and
ii. one or more MALDI matrix additives selected from the group consisting of
(a) a MS-compatible solubilizer,
(b) a MS-compatible sorbent, and
(c) a MS-compatible buffer;
b. co-precipitating said analyte with the MALDI matrix, thus generating analyte:matrix crystals;
c. subjecting said analyte:matrix crystals to laser irradiation, thus generating analyte ions; and
d. detecting and quantifying said analyte ions,
thus generating a MALDI-MS spectrum of said analyte.

103. The method of claim 102, wherein said analyte is a hydrophilic molecule.

104. The method of claim 102, wherein said analyte is a hydrophobic molecule.

105. The method of claim 102, wherein at least one of said one or more MALDI matrix additives is a MS-compatible solubilizer.

106. The method of claim 102, wherein the analyte is a molecule selected from the group consisting of other proteins, peptides, DNA, RNA, oligonucleotides, nucleic acids, oligosaccharides, polysaccharides, lipids, phospholipids, synthetic polymers, small organic molecules, and complexes or combinations of any of the above.

107. A method of obtaining a MS-MALDI spectrum of a protein analyte, comprising:
a. contacting a protein analyte with, in either order or in combination,
i. a MALDI matrix; and
ii. one or more MALDI matrix additives selected from the group consisting of
(a) a MS-compatible solubilizer,
(b) a MS-compatible sorbent, and
(c) a MS-compatible buffer;
b. co-precipitating said protein analyte with said MALDI matrix, thus generating analyte:matrix crystals;
c. subjecting said analyte:matrix crystals to laser irradiation, thus generating protein analyte ions; and
d. detecting and quantifying protein analyte ions,
thus generating a MALDI-MS spectrum of said protein analyte.

108. A method of determining one or more amino acid sequences of a protein analyte, said method comprising:
a. contacting said protein analyte with, in any order or combination,
i. a MALDI matrix;
ii. one or more MALDI matrix additives selected from the group consisting of
(a) a MS-compatible solubilizer,
(b) a MS-compatible sorbent, and
(c) a MS-compatible buffer; and
iii. at least one protease;
thus generating one or more peptides;
b. co-precipitating said one or more peptides with said MALDI matrix, thus generating analyte:matrix crystals;
c. subjecting said analyte:matrix crystals to laser irradiation, thus generating peptide analyte ions;
d. detecting and quantifying said peptide analyte ions, thus generating a MALDI-MS spectrum of said one or more peptides; and
e. using said MALDI-MS spectrum to determine the amino acid sequences of said one or more peptides;
wherein the amino acid sequence of one of said peptides is an amino acid sequence of said protein analyte.

109. A method for identifying an amino acid sequence of a protein analyte that binds to a ligand, said method comprising:
a. contacting a first sample comprising said protein analyte with, in any order or combination,
i. a MALDI matrix; and
ii. one or more MALDI matrix additives selected from the group consisting of
(a) a MS-compatible solubilizer,
(b) a MS-compatible sorbent, and
(c) a MS-compatible buffer;
b. contacting a second sample comprising said protein analyte with, in any order or combination,
i. a MALDI matrix;
ii. one or more MALDI matrix additives selected from the group consisting of
(a) a MS-compatible solubilizer,
(b) a MS-compatible sorbent, and
(c) a MS-compatible buffer; and
iii. said ligand;
c. independently contacting said first and second samples with a protease, thus generating a first set of one or more peptides and a second set of one or more peptides;
d. independently co-precipitating said first set and said second set of one or more peptides with said MALDI matrix, thus generating a first and second analyte:matrix crystal;
e. independently subjecting said first and second analyte:matrix crystals to laser irradiation, thus generating a first set and a second set of one or more peptide analyte ions;
f. independently detecting and quantifying said first set and said second set of one or more peptide analyte ions, thus generating a MALDI-MS spectrum for each of said first and said second set of peptides and; and
g. using said MALDI-MS spectra to determine the amino acid sequences of said first set and said second set one or more peptides;
wherein an amino acid sequence derived from said first set of peptides that is depleted or absent in the amino acid sequences derived from said second set of peptides is an amino acid sequence of said protein analyte that binds to said ligand.

110. A method for identifying an amino acid sequence of a protein analyte that binds to a ligand, said method comprising:
a. contacting said protein analyte with, in any order or combination,
i. a MALDI matrix;
ii. one or more MALDI matrix additives selected from the group consisting of
(a) a MS-compatible solubilizer,
(b) a MS-compatible sorbent, and
(c) a MS-compatible buffer;
iii. said ligand; and
iv. one or more cross-linkers,
under conditions and for a length of time sufficient for the concentration of free ligand to decrease by at least about 10%, thus generating a composition comprising cross-linked protein:ligand complexes;
b. contacting said cross-linked protein:ligand complexes with a protease, thus generating a set of one or more peptides comprising at least one cross-linked peptide;
c. co-precipitating said set of one or more peptides with said MALDI matrix, thus generating analyte:matrix crystals;
d. subjecting said analyte:matrix crystals to laser irradiation, thus generating a set of one or more peptide analyte ions;
e. detecting and quantifying said one or more peptide analyte ions, thus generating a MALDI-MS spectrum;
f. using said MALDI-MS spectra to determine the amino acid sequences of one or more cross-linked peptides;
wherein an amino acid sequence of one or more of said cross-linked peptides is an amino acid sequence of said protein analyte that binds to said ligand.

111. A method for identifying an amino acid sequence of a protein analyte that is chemically modified by a protein-modifying enzyme, said method comprising:
a. contacting said protein analyte with, in any order or combination,
i. a MALDI matrix;
ii. one or more MALDI matrix additives selected from the group consisting of
(a) a MS-compatible solubilizer,
(b) a MS-compatible sorbent, and
(c) a MS-compatible buffer;
iii. said ligand;
iv. said enzyme; and
v. a protease,
under conditions and for a length of time sufficient for:
(a) the concentration of a substrate for said enzyme to decrease by at least about 10%, and/or the concentration of a product of said enzyme to increase by at least about 10%; and
(b) the concentration of a substrate for said protease to decrease by at least about 10%, and/or the concentration of a product of said protease to increase by at least about 10%,
thus generating a set of chemically modified peptides;
b. co-precipitating said chemically modified peptides with said MALDI matrix, thus generating analyte:matrix crystals;
c. subjecting said analyte:matrix crystals to laser irradiation, thus generating a set of one or more chemically modified peptide analyte ions;
d. detecting and quantifying said one or more chemically modified peptide analyte ions, thus generating a MALDI-MS spectrum;
e. using said MALDI-MS spectra to determine the amino acid sequences of one or more of said chemically modified peptides;
wherein an amino acid sequence of one or more chemically modified peptides is an amino acid sequence of said protein analyte that is chemically modified by said enzyme.

112. The method of claim 111, wherein said protein-modifying enzyme is selected from the group consisting of one or more kinases, one or more phosphatases, one or more glycosylases, one or more deglycosylases, and combinations and complexes thereof.

113. A method of increasing the extent of amino acid sequence coverage of a protein analyte or a region thereof, as determined by MS-MALDI, said method comprising:
a. contacting said protein analyte with, in any order or combination,
i. a MALDI matrix;
ii. an effective amount of one or more MALDI matrix additives selected from the group consisting of
(a) a MS-compatible solubilizer,
(b) a MS-compatible sorbent, and
(c) a MS-compatible buffer; and
iii. at least one protease,
thus generating a set of peptides;
b. co-precipitating said one or more peptides with said MALDI matrix, thus generating analyte:matrix crystals;
c. subjecting said analyte:matrix crystals to laser irradiation, thus generating peptide analyte ions;
d. detecting and quantifying said peptide analyte ions, thus generating a MALDI-MS spectrum of said one or more peptides; and
e. using said MALDI-MS spectrum to determine the amino acid sequences of said one or more peptides,
wherein an effective amount of said one or more MALDI matrix additives increases the extent of amino acid sequence coverage of said protein analyte or a region thereof.

114. The method of claim 113, wherein an effective amount of said one or more MALDI matrix additives increases the extent of amino acid sequence coverage results in (a) an increase of at least one amino acid in the length of an amino acid sequence of at least one of said peptides, and/or (b) the detection of one or more peptides that are not detected in the absence of said one or more MALDI matrix additives.

115. The method of claim 113, wherein said MALDI matrix additive is a MS-compatible solubilizer.

116. The method of claim 115, wherein said increase in the extent of amino acid sequence coverage extends the sequence coverage of a hydrophobic core region of a globular soluble protein.

117. The method of claim 115, wherein said increase in the extent of amino acid sequence coverage extends the sequence coverage of a membrane protein.

118. The method of claim 115, wherein said increase in the extent of amino acid sequence coverage extends the sequence coverage of a transmembrane domain.

119. The method of claim 113, wherein said MALDI matrix additive is a MS-compatible sorbent.

120. The method of claim 113, wherein said MALDI matrix additive is a MS-compatible buffer.

121. A method of identifying variants, isoforms and homologs of a protein of interest, wherein said method comprises
a. determining one or more amino acid sequences of an uncharacterized protein analyte, said determining comprising:
i. contacting said uncharacterized protein analyte with, in any order or combination,
1. a MALDI matrix;
2. one or more an effective amount of one or more MALDI matrix additives selected from the group consisting of
(a) a MS-compatible solubilizer,
(b) a MS-compatible sorbent, and
(c) a MS-compatible buffer; and
3. at least one protease;
thus generating one or more peptides;
ii. co-precipitating said one or more peptides with said MALDI matrix, thus generating analyte:matrix crystals;
iii. subjecting said analyte:matrix crystals to laser irradiation, thus generating peptide analyte ions;
iv. detecting and quantifying said peptide analyte ions, thus generating a MALDI-MS spectrum of said one or more peptides; and
v. using said MALDI-MS spectrum to determine the amino acid sequences of said one or more peptides of said uncharacterized protein; and
b. determining the homology of an amino acid sequence of said one or more peptides of said uncharacterized protein to amino acid sequences from said protein of interest,
wherein an uncharacterized protein that comprises amino acid sequences that are identical or homologous to amino acid sequences from said protein of interest is a variants, isoform or homolog of said protein of interest.

122. The method of claim 121, wherein at least one of said amino acid sequences of said uncharacterized protein has greater than about 50% homology to an amino acid sequence in said protein of interest.

123. The method of claim 121, wherein at least one of said amino acid sequences of said uncharacterized protein has greater than about 90% homology to an amino acid sequence in said protein of interest.

124. The method of claim 121, wherein at least one of said amino acid sequences of said uncharacterized protein is identical to an amino acid sequence in said protein of interest.

125. The method of any of claims 121-124, wherein said amino acid sequence in said protein of interest is from 20 to about 500 amino acids in length.

126. The method of any of claims 121-124, wherein said amino acid sequence in said protein of interest is from 20 to about 100 amino acids in length.

127. The method of any of claims 121-124, wherein said homology is determined by a software program in silico.

128. The method of any of claims 108, 109, 110, 111, 113 or 121, wherein said protease is selected from the group consisting of TEV protease, trypsin, chymotrypsin, elastase, Endoproteinase Arg-C, Endoproteinase Asp-N, Endoproteinase Glu-C, Endoproteinase Lys-C, Aminopeptidase M, Carboxypeptidase-Y and pronase.

129. A method of decreasing the amount of ion adducts of an analyte in MALDI-MS, comprising
a. contacting said analyte with, in either order or in combination,
i. a MALDI matrix; and
ii. one or more MALDI matrix additives selected from the group consisting of
(a) a MS-compatible solubilizer,
(b) a MS-compatible sorbent, and
(c) a MS-compatible buffer; and
b. co-precipitating said analyte with said MALDI matrix, thus generating analyte:matrix crystals,
wherein an effective amount of said one or more MS-compatible compositions decreases the signal from one or more adduct cluster peaks in a MALDI-MS spectrum.

130. The method of claim 129, wherein said ions are cations.

131. The method of claim 130, wherein said cations are monovalent cations.

132. The method of claim 131, wherein said monovalent cations are selected from the group consisting of Sodium (Na⁺), Potassium (K⁺), Rubidium (Rb⁺), Lithium (L⁺i), Cesium (Cs⁺), Francium (Fr⁺), Thallium (Tl⁺), ammonium ion (NH₄⁺), and guanium ion [C(NH₂)₃⁺].

133. The method of claim 129, wherein said analyte is a protein analyte.

134. The method of claim 129, wherein the M-1 adduct cluster peak of a molecule that forms adduct with ions is decreased by at least about 50% in the presence of an effective amount of said MALDI matrix additive.

135. The method of claim 129, wherein the M-1 adduct cluster peak of a molecule that forms adduct with ions is decreased by at least about 95% in the presence of an effective amount of said MALDI matrix additive.

136. The method of claim of claim 129, wherein said molecule that forms adduct with ions is bradykinin.

137. The method of claim 129, further comprising washing said analyte:matrix crystals with one or more MALDI matrix additives selected from the group consisting of
a. a MS-compatible solubilizer,
b. a MS-compatible sorbent, and
c. a MS-compatible buffer.

138. The method of any of claims 101, 102, 107-111, 113, 121 or 129, wherein at least some of said one or more MS-compatible solubilizers co-precipitates with said analyte and matrix, and said analyte:matrix crystal comprises one or more of said MALDI matrix additives.

139. The method of claim 138, wherein at least about 75% of said MALDI matrix additive co-precipitates with said analyte and matrix and is present in said analyte:matrix crystal.

140. The method of claim 138, wherein said MALDI matrix additive is a MS-compatible solubilizer.

141. The method of claim 140, wherein said MS-compatible solubilizer comprises one or more MS-compatible detergents.

142. The method of claim 141, wherein at least one of said one or more MS-compatible detergents is present in said analyte:matrix crystal at a concentration that is from about 50% to about 500% of its CMC.

143. The method of claim 141, wherein at least one of said one or more MS-compatible detergents is present in said analyte:matrix crystal at a concentration that is from about 75% to about 150% of its CMC.

144. The method of any of claims 105, 107-111, 113, 121 and 133, wherein said protein analyte is a synthetic oligopeptide.

145. The method of any of claims 105, 107-111, 113, 121 and 133, wherein said protein analyte is a polypeptide.

146. The method of any of claims 105, 107-111, 113, 121 and 133, wherein said protein analyte comprises two or more polypeptide chains.

147. The method of any of claims 105, 107-111, 113, 121 and 133, wherein said protein analyte is a protein complex.

148. The method of claim 147, wherein said protein complex comprises a protein of interest and one or more other molecules selected from the group consisting of one or more other proteins, peptides, DNA, RNA, oligonucleotides, nucleic acids, oligosaccharides, polysaccharides, lipids, phospholipids, synthetic polymers, small organic molecules, and complexes or combinations of any of the above.

149. The method of claim 134, wherein said protein analyte is a membrane protein.

150. The method of any of claims 105, 107-111, 113, 121 and 133, wherein said protein analyte comprises a transmembrane domain.

151. The method of any of claims 105, 107-111, 113, 121 and 133, wherein said protein analyte is an ion channel or a transporter.

152. The method of claim 137, wherein said protein is a ligand-gated ion channel selected from the group consisting of a serotonin receptor, a gamma-aminobutyric acid receptor, a glycine receptor, a glutamate-gated chloride channel, a glutamate receptor, an ATP-gated channel, and an NMDA receptor.

153. The method of claim 139, wherein said membrane protein is a transporter selected from the group consisting of a serotonin transporter and an ATP transporter.

154. The method of any of claims 105, 107-111, 113, 121 and 133, wherein said protein analyte comprises a binding site for ligand.

155. The method of claim 154, wherein said ligand is a biomolecule.

156. The method of claim 155, wherein said ligand is a biomolecule selected from the group consisting of an antibody, an agonist, an antagonist, an allosteric modulator, a phospholipid, a cholesterol or modified cholesterol molecule, a fatty acid, a steroid, a hormone, a volatile anesthetic, a fluxing ion, an ion cofactor and complexes and combinations thereof.

157. The method of claim 154, wherein said ligand is a synthetic compound.

158. The method of claim 157, wherein said synthetic compound is a drug, a drug candidate or a lead compound.

159. The method of any of claims 105, 107-111, 113, 121 and 133, wherein said protein analyte has a homology of >50% to one or more subunits of a nicotinic acetylcholine receptor.

160. The method of claim 159, wherein said nicotinic acetylcholine receptor is the nicotinic acetylcholine receptor of Torpedo californica.

161. The method of any of claims 105, 107-111, 113, 121 and 133, wherein said protein analyte has a homology of >50% to one or more of the sequences selected from the group consisting of SEQ ID NO:1, SEQ ID NO:2, SEQ ID NO:3, and SEQ ID NO:4.

162. The method of any of claims 105, 107-111, 113, 121 and 133, wherein said protein analyte comprises a domain or region having a homology of >50% with a sequence within a domain or region of nicotinic acetylcholine receptor of Torpedo californica.

163. The method of claim 162, wherein said domain or region is selected from the group consisting of the M2 pore forming transmembrane domain, the M2-M3 linker region and various regions of the N-terminal domain associated with agonist binding.

164. The method of claim 163, wherein said sequence within a domain or region is selected from the group consisting of SEQ ID NO:5, SEQ ID NO:6, and SEQ ID NO:7.

165. The method of any of claims 105, 107-111, 113, 121 and 129, wherein said MS-compatible solubilizer comprises ASB-C8Ø, Octyl-beta-D-1-thioglucopyranoside, n-Dodecanoylsucrose, and SB14.

166. The method of claim 165, wherein the concentration of ASB-C8Ø is from about 0.025 mM; the concentration of Octyl-beta-D-1-thioglucopyranoside is about 10 mM; and the concentration of n-Dodecanoylsucrose is about 0.76 mM; and the concentration of SB14 is about 0.2 mM.

167. The method of any of claims 105, 107-111, 113, 121 and 129, wherein said MS-compatible solubilizer comprises a non-detergent surfactant.

168. The method of claim 167, wherein said non-detergent surfactant is NDSB-201.

169. The method of claim 168, wherein said NDSB-201 is at a concentration of from about 100 mM to about 500 mM.

170. The method of any of claims 105, 107-111, 113, 121 and 129, wherein said MALDI matrix is selected from the group consisting of alpha-cyano-4-hydroxycinnamic acid, sinapinic acid, 2,5-dihydroxybenzoic acid, nor-harmane and glycerol.

171. The method of any of claims 105, 107-111, 113, 121 and 129, wherein said MS-compatible sorbent is selected from the group consisting of silica; alumina; titanium; tin; germanium oxide; an indium tin oxide; a metal oxide; a chloride; a sulfate; a phosphate; a carbonate; a fluoride; a polymer-based oxide, chloride, sulfate, carbonate, phosphate or fluoride; diatomaceous earth; graphite or activated charcoal; gold; and activated gold.

172. The method of any of claims 105, 107-111, 113, 121 and 129, wherein said one or more MS-compatible sorbents is a resin.

173. The method of claim 172, wherein said resin is selected from the group consisting of LiChrosorb®, LiChrospher®, LiChroprep®, LiChroprep® and Purospher®.

174. The method of claim 172, wherein said resin is selected from the group consisting of LiChrosorb® 5 μm, 5 μm RPB, 5 μm RP18, LiChrosorb® 5 μm RP-Select B, LiChrosorb® 5 μm DIOL, LiChrosorb® 10 μm RP18, LiChrosorb® 10 μm RP8, LiChrosorb® 10 μm RP18, LiChrosorb® 5 μm Si60 and Silica Gel 60 RP-18.

175. The method of any of claims 105, 107-111, 113, 121 and 129, wherein said one or more MS-compatible sorbents is a composition comprising particles.

176. The method of claim 175, wherein said composition comprising particles has a D90/D10<about 2.0.

177. The method of claim 175, wherein said particles have at least one dimension greater than 10 microns.

178. The method of any of claims 105, 107-111, 113, 121 and 129, wherein said MS-compatible buffer is a morpholino-sulfonic acid.

179. The method of any of claims 105, 107-111, 113, 121 and 129, wherein said MS-compatible buffer is selected from the group consisting of MES, MOBS, MOPS, and MOPSO.

180. The method of any of claims 105, 107-111, 113, 121 and 129, wherein said MS-compatible buffer has the structure 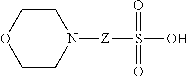
Wherein Z═[CH₂]ₐ—[CH—OH]_{b}—[CH₂]_{c}, and wherein:
a=0 to 25,
b=0 to 25,
c=0 to 25,
with the exception that, if b=0, a and c cannot both be 0.

181. A kit comprising one or more MALDI matrix additives selected from the group consisting of
a. a MS-compatible solubilizer,
b. a MS-compatible sorbent, and
c. a MS-compatible buffer.

182. The kit of claim 181, further comprising one or more matrix compositions.

183. The kit of claim 182, wherein said one or more matrix compositions is selected from the group consisting of alpha-cyano-4-hydroxycinnamic acid, sinapinic acid, 2,5-dihydroxybenzoic acid, nor-harmane and glycerol.

184. The kit of claim 181, further comprising one or more matrix solvents.

185. The kit of claim 184, wherein said one or more matrix solvents is selected from the group consisting of 0.1% trifluoroacetic acid and 100% acetonitrile.

186. The kit of claim 181, further comprising one or more chaotropes.

187. The kit of claim 186, wherein said one or more chaotropes are selected from the group consisting of urea, thiourea and guanidine chloride.

188. The kit of claim 181, further comprising one or more enzymes.

189. The kit of claim 188, wherein said enzyme is a protease.

190. The kit of claim 189, wherein said protease is wherein said protease is selected from the group consisting of TEV protease, trypsin, chymotrypsin, elastase, Endoproteinase Arg-C, Endoproteinase Asp-N, Endoproteinase Glu-C, Endoproteinase Lys-C, Aminopeptidase M, Carboxypeptidase-Y and pronase.

191. The kit of claim 181, further comprising one or more buffers.

192. The kit of claim 181, further comprising one or more cross-linkers.

193. The kit of claim 181, further comprising one or more standards, controls or calibrants.

194. The kit of claim 181, further comprising a product manual that describes storage conditions and one or more experimental protocols.

195. The kit of claim 194, wherein said experimental protocols are selected from the group consisting of a protocol for direct analysis and calibration of intact hydrophobic proteins, a buffer exchange protocol, and a trypsin digestion protocol.

196. The kit of claim 181, wherein said MALDI matrix additive is a MS-compatible solubilizer.

197. The kit of claim 196, wherein at least one of said MS-compatible solubilizers comprises a compound selected from the group consisting of ASB-C8Ø, Octyl-beta-D-1-thioglucopyranoside, n-Dodecanoylsucrose, and SB14.

198. The kit of claim 196, wherein at least one of said MS-compatible solubilizers comprises ASB-C8Ø, Octyl-beta-D-1-thioglucopyranoside, n-Dodecanoylsucrose, and SB14.

199. The kit of claim 198, wherein the concentration of ASB-C8Ø is from about 0.01 to about 0.5 mM; the concentration of Octyl-beta-D-1-thioglucopyranoside is from about 1 to about 50 mM; the concentration of n-Dodecanoylsucrose is from about 0.1 to about 10 mM; and the concentration of SB14 is from about 0.05 to about 1 mM.

200. The kit of claim 198, wherein the concentration of ASB-C8Ø is from about 0.025 mM; the concentration of Octyl-beta-D-1-thioglucopyranoside is about 10 mM; and the concentration of n-Dodecanoylsucrose is about 0.76 mM; and the concentration of SB14 is about 0.2 mM.

201. The kit of claim 186, wherein at least one of said MS-compatible solubilizers comprises a non-detergent sulfobetaine.

202. The kit of claim 201, wherein said non-detergent sulfobetaine is selected from the group consisting of NDSB-195, NDSB-201, NDSB-211, NDSB-221, NDSB-223, and NDSB-256.

203. The kit of claim 201, wherein said non-detergent sulfobetaine is NDSB-201.

204. The kit of claim 203, wherein said NSBD-201 is present at a concentration of about 500 mM.

205. The kit of claim 182, wherein said MALDI matrix additive is in the form of a concentrated stock solution.

206. The kit of claim 205, wherein said concentrated stock solution has a concentration selected from the group consisting of 1.5×, 2×, 3×, 4×, 5×, 10×, 15×, 25×, 5× and 100×.

207. The kit of claim 181, further comprising a product manual that describes storage conditions and one or more experimental protocols.

208. The kit of claim 181, further comprising at least one empty container.

209. A kit for MALDI-TOF MS comprising:
a. A container comprising a solution of ASB-C8Ø, Octyl-beta-D-1-thioglucopyranoside, n-Dodecanoylsucrose and SB14;
b. A container comprising NDSB-201;
c. A container comprising one or more molecular weight standards;
d. A container comprising sinapinic acid;
e. A container comprising alpha-cyano-4-hydroxycinnamic acid;
f. A container comprising trifluoroacetic acid; and
g. A container comprising acetonitrile.

210. A kit for MALDI-MS comprising:
a. A container comprising 10 ml of a solution of ASB-C8Ø at 0.125 mM, Octyl-beta-D-1-thioglucopyranoside at 50 mM, n-Dodecanoylsucrose at 3.8 mM, and SB l4 at 1 mM;
b. A container comprising 25 ml of 500 mM NDSB-201;
c. A container comprising 25 μL of 90 kDa InvitroMass protein standard;
d. A container comprising 20 mg of sinapinic acid;
e. A container comprising 20 mg of alpha-cyano-4-hydroxycinnamic acid;
f. A container comprising 20 ml of 0.1% trifluoroacetic acid; and
g. A container comprising 1 ml of 100% acetonitrile.

211. The kit of claim 181, wherein said MS-compatible sorbent is selected from the group consisting of silica; alumina; titanium; tin; germanium oxide; an indium tin oxide; a metal oxide; a chloride; a sulfate; a phosphate; a carbonate; a fluoride; a polymer-based oxide, chloride, sulfate, carbonate, phosphate or fluoride; diatomaceous earth; graphite or activated charcoal; gold; and activated gold.

212. The kit of claim 181, wherein said MS-compatible sorbent is silica.

213. The kit of claim 181, wherein said one or more MS-compatible sorbents is a resin.

214. The kit of claim 213, wherein said resin is selected from the group consisting of LiChrosorb®, LiChrospher®, LiChroprep®, LiChroprep® and Purospher®.

215. The kit of claim 203, wherein said resin is selected from the group consisting of LiChrosorb® 5 μm, 5 μm RP8, 5 μm RP18, LiChrosorb® 5 μm RP-Select B, LiChrosorb® 5 μm DIOL, LiChrosorb® 10 μm RP18, LiChrosorb® 10 μm RP8, LiChrosorb® 10 μm RP18, LiChrosorb® 5 μm Si60 and Silica Gel 60 RP-18.

216. The kit of claim 181, wherein said one or more MS-compatible sorbents is a composition comprising particles.

217. The kit of claim 216, wherein said composition comprising particles has a D90/D10≦about 2.0.

218. The kit of claim 216, wherein said particles comprise silica.

219. The kit of claim 216, wherein said particles have at least one dimension >1 microns.

220. The kit of claim 181, wherein said MALDI matrix additive is a MS-compatible buffer.

221. The kit of claim 220, wherein said MS-compatible buffer is a morpholino-sulfonic acid.

222. The kit of claim 220, wherein said MS-compatible buffer has the structure 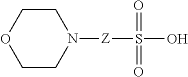
Wherein Z═[CH₂]ₐ—[CH—OH]_{b}—[CH₂]_{c}, and wherein:
a=0 to 25,
b=0 to 25,
c=0 to 25,
with the exception that, if b=0, a and c cannot both be 0.

223. The kit of claim 222, wherein b=0 and c=0.

224. The kit of claim 220, wherein said MS-compatible buffer is selected from the group consisting of MES, MOBS, MOPS, and MOPSO.

225. A solid support comprising or coated with one or more MALDI matrix additives selected from the group consisting of
a. a MS-compatible solubilizer,
b. a MS-compatible sorbent, and
c. a MS-compatible buffer.

226. The solid support of claim 226, wherein said solid support is selected from the group consisting of a bead, a monolithic column or chip surface and the interior of chromatographic tubing.

227. The solid support of claim 226, wherein said solid support is a MS target surface.

228. A MALDI-MS target surface coated with one or more MALDI matrix additives selected from the group consisting of
a. a MS-compatible solubilizer,
b. a MS-compatible sorbent, and
c. a MS-compatible buffer.

229. A method of coating a substrate, comprising contacting said substrate to one or more MALDI matrix additives selected from the group consisting of
a. a MS-compatible solubilizer,
b. a MS-compatible sorbent, and
c. a MS-compatible buffer.

230. The method of claim 230, wherein said contacting comprises electrospraying.

231. The method of claim 230, wherein said substrate is the internal and/or external surface of a bead.

232. The method of claim 230, wherein said substrate is a MALDI-MS target surface.

233. An analyte:matrix crystal comprising an analyte, a MALDI matrix and one or more MALDI matrix additives selected from the group consisting of
a. a MS-compatible solubilizer,
b. a MS-compatible sorbent, and
c. a MS-compatible buffer.

234. The analyte:matrix crystal of claim 234, wherein said MALDI matrix is selected from the group consisting of alpha-cyano-4-hydroxycinnamic acid, sinapinic acid, 2,5-dihydroxybenzoic acid, nor-harmane and glycerol.

235. The analyte:matrix crystal of claim 234, wherein said analyte is a protein.

236. The analyte:matrix crystal of claim 234, wherein said MALDI matrix additive is a MS-compatible solubilizer.

237. The analyte:matrix crystal of claim 237, wherein said MS-compatible solubilizer is a MS-compatible detergent.

238. A composition comprising a MALDI matrix and a MALDI matrix additive, wherein said composition can be stably stored at −20° C. for 1-36 months before being used to make analyte:matrix crystals.

239. A composition comprising a MALDI matrix and a MALDI matrix additive, wherein said composition can be stably stored at −20° C. for about 8 months before being used to make analyte:matrix crystals.

240. A composition comprising a MALDI matrix and a MALDI matrix additive, wherein said composition can be stably stored at 4° C. for 1-24 months before being used to make analyte:matrix crystals.

241. A composition comprising a MALDI matrix and a MALDI matrix additive, wherein said composition can be stably stored at 4° C. for about 8 months before being used to make analyte:matrix crystals.

242. A composition comprising a MALDI matrix and a MALDI matrix additive, wherein said composition can be stably stored at 37° C. for 1-24 months before being used to make analyte:matrix crystals.

243. A composition comprising a MALDI matrix and a MALDI matrix additive, wherein said composition can be stably stored at 37° C. for about 4 weeks before being used to make analyte:matrix crystals.

244. A composition comprising a MALDI matrix and a MALDI matrix additive, wherein said composition can be stably stored at 37° C. for about 8 days before being used to make analyte:matrix crystals.

245. The MS-compatible solubilizer of claim 1 comprising at least one non-detergent surfactant.

246. The MS-compatible solubilizer of claim 245, wherein the at least one of said one or more non-detergent surfactants comprises a non-detergent sulfobetaine.

247. The MS-compatible solubilizer of claim 246, wherein said non-detergent sulfobetaine is selected from the group consisting of NDSB-195, NDSB-201, NDSB-211, NDSB-221, NDSB-223, and NDSB-256.

248. The MS-compatible solubilizer of claim 246, wherein said non-detergent sulfobetaine is NDSB-201.

249. The MS-compatible solubilizer of claim 248, wherein said NSBD-201 is at a concentration of from about 5 mM to about 1 M.

250. The MS-compatible solubilizer of claim 249, wherein said NDSB-201 is at a concentration of from about 10 mM to about 0.8 M.

251. The MS-compatible solubilizer of claim 248, wherein said NDSB-201 is at a concentration of from about 50 mM to about 700 mM.

252. The MS-compatible solubilizer of claim 248, wherein said NSBD-201 is at a concentration of about 100 mM to about 600 mM.

253. The MS-compatible solubilizer of claim 248, wherein said NSBD-201 is at a concentration of about 250 mM to about 500 mM.

254. The MS-compatible solubilizer of claim 246, wherein said non-detergent sulfobetaine is NDSB-256.

255. The MS-compatible solubilizer of claim 254, wherein said NSBD-256 is at a concentration of from about 10 mM to about 1 M.

256. The MS-compatible solubilizer of claim 254, wherein said NDSB-256 is at a concentration of from about 20 mM to about 0.8 M.

257. The MS-compatible solubilizer of claim 254, wherein said NDSB-256 is at a concentration of from about 50 mM to about 750 mM.

258. The MS-compatible solubilizer of claim 254, wherein said NSBD-256 is at a concentration of about 125 mM.

259. The MS-compatible solubilizer of claim 254, wherein said NSBD-256 is at a concentration of about 250 mM.
